(19) United States
(12) Patent Application Publication
(10) Pub. No.: US 2024/0209062 A1
(43) Pub. Date: Jun. 27, 2024

(54) POLYPEPTIDES WITH ALTERED BINDING TO NEONATAL FC RECEPTOR (FCRN) AND METHODS OF USE

(71) Applicant: Invetx, Inc., Boston, MA (US)

(72) Inventor: William BRONDYK, Mansfield, MA (US)

(21) Appl. No.: 18/396,160

(22) Filed: Dec. 26, 2023

Related U.S. Application Data
(60) Provisional application No. 63/598,622, filed on Nov. 14, 2023.
(60) Provisional application No. 63/477,267, filed on Dec. 27, 2022.

(51) Int. Cl. C07K 14/735; A61K 38/00; C12N 15/63

(52) U.S. Cl. CPC C07K 14/70535; C12N 15/63; A61K 38/00

(57) ABSTRACT

Provided are compositions for increasing the half-life of a polypeptide or polypeptides in a feline and methods of their use. The compositions involve variant feline IgG Fc regions.

### SEQUENCE LISTING

The instant application contains a Sequence Listing which has been submitted electronically in XML format and is hereby incorporated by reference in its entirety. Said XML copy, created on Dec. 11, 2023, is named “51682-007003_Sequence_Listing_12_11_23” and is 165,160 bytes in size.

### FIELD

This disclosure relates generally to polypeptides (e.g., fusion polypeptides such as polypeptide-Fc region fusions; or binding molecules such as antibodies, antigen-binding antibody fragments, or ligand-binding portions of receptor-Fc fusions) that have increased half-life in felines compared to their wild type counterparts.

### BACKGROUND

The Fc region of antibodies plays a number of functional roles, including, but not limited to, protecting the antibody from degradation through the lysosomal pathway and mediating antibody effector functions. With the increasing use of feline antibodies as therapeutic agents, there has been an enhanced focus on not just selecting an optimal antibody or antibody fragment (e.g., Fab), but also combining it with an appropriate Fc for desired half-life and effector functions.

There is little guidance in the art relating to increasing half-life of polypeptide therapeutics (e.g., antibodies) for use in cats. Therefore, there is a need for Fc region variants that improve the serum persistence of polypeptides (e.g., antibodies) in felines.

### SUMMARY

Provided herein are feline Fc regions (e.g., feline IgG Fc region variants) or feline FcRn binding fragments thereof that are useful in therapeutic polypeptides. For example, provided herein are polypeptides that include feline IgG Fc region variants, in which the feline IgG Fc region variants have increased half-life in felines compared to their wild type counterparts.

In a first aspect, the invention features a polypeptide comprising a feline IgG Fc region variant, wherein the feline IgG Fc region variant comprises (i) Tyr at a position that corresponds to amino acid position 252 of a wild type feline IgG, and (ii) at least one amino acid substitution at a position selected from the group consisting of:
(i) a position that corresponds to amino acid position 286 of a wild type feline IgG;
(ii) a position that corresponds to amino acid position 301 of a wild type feline IgG;
(iii) a position that corresponds to amino acid position 309 of a wild type feline IgG, wherein the amino acid substitution is Asp or Val;
(iv) a position that corresponds to amino acid position 377 of a wild type feline IgG; and
(v) a position that corresponds to amino acid position 392 of a wild type feline IgG;
wherein the amino acid positions are based on EU numbering, and wherein the polypeptide has increased binding affinity to feline FcRn compared to an Fc domain of the wild type feline IgG.

In some embodiments, the polypeptide comprises Asp or Glu at the amino acid position that corresponds to amino acid position 286 of the wild type feline IgG.

In some embodiments, the polypeptide comprises Leu, Tyr, or Val at the amino acid position that corresponds to amino acid position 301 of the wild type feline IgG.

In some embodiments, the polypeptide comprises Leu or Tyr at the amino acid position that corresponds to amino acid position 377 of the wild type feline IgG.

In some embodiments, the polypeptide comprises Asp or Glu at the amino acid position that corresponds to amino acid position 392 of the wild type feline IgG.

In some embodiments, the polypeptide comprises:
(i) Tyr at the amino acid position that corresponds to amino acid position 252 of the wild type feline IgG and Asp at the amino acid position that corresponds to amino acid position 286 of the wild type feline IgG
(ii) Tyr at the amino acid position that corresponds to amino acid position 252 of the wild type feline IgG and Glu at the amino acid position that corresponds to amino acid position 286 of the wild type feline IgG
(iii) Tyr at the amino acid position that corresponds to amino acid position 252 of the wild type feline IgG and Leu at the amino acid position that corresponds to amino acid position 301 of the wild type feline IgG;
(iv) Tyr at the amino acid position that corresponds to amino acid position 252 of the wild type feline IgG and Tyr at the amino acid position that corresponds to amino acid position 301 of the wild type feline IgG;
(v) Tyr at the amino acid position that corresponds to amino acid position 252 of the wild type feline IgG and Val at the amino acid position that corresponds to amino acid position 301 of the wild type feline IgG;
(vi) Tyr at the amino acid position that corresponds to amino acid position 252 of the wild type feline IgG and Asp at the amino acid position that corresponds to amino acid position 309 of the wild type feline IgG;
(vii) Tyr at the amino acid position that corresponds to amino acid position 252 of the wild type feline IgG and Val at the amino acid position that corresponds to amino acid position 309 of the wild type feline IgG;
(viii) Tyr at the amino acid position that corresponds to amino acid position 252 of the wild type feline IgG and Leu at the amino acid position that corresponds to amino acid position 377 of the wild type feline IgG;
(ix) Tyr at the amino acid position that corresponds to amino acid position 252 of the wild type feline IgG and Tyr at the amino acid position that corresponds to amino acid position 377 of the wild type feline IgG;
(x) Tyr at the amino acid position that corresponds to amino acid position 252 of the wild type feline IgG and Asp at the amino acid position that corresponds to amino acid position 392 of the wild type feline IgG; or
(xi) Tyr at the amino acid position that corresponds to amino acid position 252 of the wild type feline IgG and Glu at the amino acid position that corresponds to amino acid position 392 of the wild type feline IgG.

In another aspect, the invention features a polypeptide comprising a feline IgG Fc region variant, wherein the feline IgG Fc region variant comprises (i) Met at a position that corresponds to amino acid position 252 of a wild type feline IgG, and (ii) at least one amino acid substitution at a position selected from the group consisting of:
(i) a position that corresponds to amino acid position 286 of a wild type feline IgG;
(ii) a position that corresponds to amino acid position 301 of a wild type feline IgG;
(iii) a position that corresponds to amino acid position 309 of a wild type feline IgG, wherein the amino acid substitution is Asp or Val;
(iv) a position that corresponds to amino acid position 311 of a wild type feline IgG, wherein the amino acid substitution is Val;
(v) a position that corresponds to amino acid position 377 of a wild type feline IgG; and
(vi) a position that corresponds to amino acid position 392 of a wild type feline IgG;
wherein the amino acid positions are based on EU numbering, and wherein the polypeptide has increased binding affinity to feline FcRn compared to an Fc domain of the wild type feline IgG.

In some embodiments, the polypeptide comprises Asp or Glu at the amino acid position that corresponds to amino acid position 286 of the wild type feline IgG.

In some embodiments, the polypeptide comprises Leu, Tyr, or Val at the amino acid position that corresponds to amino acid position 301 of the wild type feline IgG.

In some embodiments, the polypeptide comprises Val at the amino acid position that corresponds to amino acid position 311 of the wild type feline IgG.

In some embodiments, the polypeptide comprises Leu or Tyr at the amino acid position that corresponds to amino acid position 377 of the wild type feline IgG.

In some embodiments, the polypeptide comprises Asp or Glu at the amino acid position that corresponds to amino acid position 392 of the wild type feline IgG.

In some embodiments, the polypeptide comprises:
(i) Met at the amino acid position that corresponds to amino acid position 252 of the wild type feline IgG and Asp at the amino acid position that corresponds to amino acid position 286 of the wild type feline IgG;
(ii) Met at the amino acid position that corresponds to amino acid position 252 of the wild type feline IgG and Glu at the amino acid position that corresponds to amino acid position 286 of the wild type feline IgG;
(iii) Met at the amino acid position that corresponds to amino acid position 252 of the wild type feline IgG and Leu at the amino acid position that corresponds to amino acid position 301 of the wild type feline IgG;
(iv) Met at the amino acid position that corresponds to amino acid position 252 of the wild type feline IgG and Tyr at the amino acid position that corresponds to amino acid position 301 of the wild type feline IgG;
(v) Met at the amino acid position that corresponds to amino acid position 252 of the wild type feline IgG and Val at the amino acid position that corresponds to amino acid position 301 of the wild type feline IgG;
(vi) Met at the amino acid position that corresponds to amino acid position 252 of the wild type feline IgG and Asp at the amino acid position that corresponds to amino acid position 309 of the wild type feline IgG;
(vii) Met at the amino acid position that corresponds to amino acid position 252 of the wild type feline IgG and Val at the amino acid position that corresponds to amino acid position 309 of the wild type feline IgG;
(viii) Met at the amino acid position that corresponds to amino acid position 252 of the wild type feline IgG and Val at the amino acid position that corresponds to amino acid position 311 of the wild type feline IgG;
(ix) Met at the amino acid position that corresponds to amino acid position 252 of the wild type feline IgG and Leu at the amino acid position that corresponds to amino acid position 377 of the wild type feline IgG;
(x) Met at the amino acid position that corresponds to amino acid position 252 of the wild type feline IgG and Tyr at the amino acid position that corresponds to amino acid position 377 of the wild type feline IgG
(xi) Met at the amino acid position that corresponds to amino acid position 252 of the wild type feline IgG and Asp at the amino acid position that corresponds to amino acid position 392 of the wild type feline IgG; or
(xii) Met at the amino acid position that corresponds to amino acid position 252 of the wild type feline IgG and Glu at the amino acid position that corresponds to amino acid position 392 of the wild type feline IgG.

In another aspect, the invention features a polypeptide comprising a feline IgG Fc region variant, wherein the feline IgG Fc region variant comprises (i) Met at a position that corresponds to amino acid position 428 of a wild type feline IgG, and (ii) at least one amino acid substitution at a position selected from the group consisting of:
(i) a position that corresponds to amino acid position 286 of a wild type feline IgG;
(ii) a position that corresponds to amino acid position 301 of a wild type feline IgG;
(iii) a position that corresponds to amino acid position 309 of a wild type feline IgG;
(iv) a position that corresponds to amino acid position 311 of a wild type feline IgG, wherein the amino acid substitution is Val;
(v) a position that corresponds to amino acid position 377 of a wild type feline IgG; and
(vi) a position that corresponds to amino acid position 392 of a wild type feline IgG;
wherein the amino acid positions are based on EU numbering, and wherein the polypeptide has increased binding affinity to feline FcRn compared to an Fc domain of the wild type feline IgG.

In some embodiments, the polypeptide comprises Asp or Glu at the amino acid position that corresponds to amino acid position 286 of the wild type feline IgG.

In some embodiments, the polypeptide comprises Leu, Tyr, or Val at the amino acid position that corresponds to amino acid position 301 of the wild type feline IgG.

In some embodiments, the polypeptide comprises Asp, Glu, or Val at the amino acid position that corresponds to amino acid position 309 of the wild type feline IgG.

In some embodiments, the polypeptide comprises Val at the amino acid position that corresponds to amino acid position 311 of the wild type feline IgG.

In some embodiments, the polypeptide comprises Leu or Tyr at the amino acid position that corresponds to amino acid position 377 of the wild type feline IgG.

In some embodiments, the polypeptide comprises Asp or Glu at the amino acid position that corresponds to amino acid position 392 of the wild type feline IgG.

In some embodiments, the polypeptide comprises:
(i) Met at the amino acid position that corresponds to amino acid position 428 of the wild type feline IgG and Asp at the amino acid position that corresponds to amino acid position 286 of the wild type feline IgG;
(ii) Met at the amino acid position that corresponds to amino acid position 428 of the wild type feline IgG and Glu at the amino acid position that corresponds to amino acid position 286 of the wild type feline IgG;
(iii) Met at the amino acid position that corresponds to amino acid position 428 of the wild type feline IgG and Leu at the amino acid position that corresponds to amino acid position 301 of the wild type feline IgG;
(iv) Met at the amino acid position that corresponds to amino acid position 428 of the wild type feline IgG and Tyr at the amino acid position that corresponds to amino acid position 301 of the wild type feline IgG
(v) Met at the amino acid position that corresponds to amino acid position 428 of the wild type feline IgG and Val at the amino acid position that corresponds to amino acid position 301 of the wild type feline IgG;
(vi) Met at the amino acid position that corresponds to amino acid position 428 of the wild type feline IgG and Asp at the amino acid position that corresponds to amino acid position 309 of the wild type feline IgG;
(vii) Met at the amino acid position that corresponds to amino acid position 428 of the wild type feline IgG and Glu at the amino acid position that corresponds to amino acid position 309 of the wild type feline IgG;
(viii) Met at the amino acid position that corresponds to amino acid position 428 of the wild type feline IgG and Val at the amino acid position that corresponds to amino acid position 309 of the wild type feline IgG;
(ix) Met at the amino acid position that corresponds to amino acid position 428 of the wild type feline IgG and Val at the amino acid position that corresponds to amino acid position 311 of the wild type feline IgG
(x) Met at the amino acid position that corresponds to amino acid position 428 of the wild type feline IgG and Leu at the amino acid position that corresponds to amino acid position 377 of the wild type feline IgG;
(xi) Met at the amino acid position that corresponds to amino acid position 252 of the wild type feline IgG and Tyr at the amino acid position that corresponds to amino acid position 377 of the wild type feline IgG;
(xii) Met at the amino acid position that corresponds to amino acid position 252 of the wild type feline IgG and Asp at the amino acid position that corresponds to amino acid position 392 of the wild type feline IgG; or
(xiii) Met at the amino acid position that corresponds to amino acid position 252 of the wild type feline IgG and Glu at the amino acid position that corresponds to amino acid position 392 of the wild type feline IgG.

In another aspect, the invention features a polypeptide comprising a feline IgG Fc region variant, wherein the feline IgG Fc region variant comprises (i) Leu at a position that corresponds to amino acid position 428 of a wild type feline IgG, and (ii) at least one amino acid substitution at a position selected from the group consisting of:
(i) a position that corresponds to amino acid position 286 of a wild type feline IgG;
(ii) a position that corresponds to amino acid position 301 of a wild type feline IgG;
(iii) a position that corresponds to amino acid position 309 of a wild type feline IgG, wherein the amino acid substitution is Asp;
(iv) a position that corresponds to amino acid position 377 of a wild type feline IgG; and
(v) a position that corresponds to amino acid position 392 of a wild type feline IgG;
wherein the amino acid positions are based on EU numbering, and wherein the polypeptide has increased binding affinity to feline FcRn compared to an Fc domain of the wild type feline IgG.

In some embodiments, the polypeptide comprises Asp at the amino acid position that corresponds to amino acid position 286 of the wild type feline IgG.

In some embodiments, the polypeptide comprises Leu, Tyr, or Val at the amino acid position that corresponds to amino acid position 301 of the wild type feline IgG.

In some embodiments, the polypeptide comprises Asp at the amino acid position that corresponds to amino acid position 309 of the wild type feline IgG.

In some embodiments, the polypeptide comprises Leu or Tyr at the amino acid position that corresponds to amino acid position 377 of the wild type feline IgG.

In some embodiments, the polypeptide comprises Asp or Glu at the amino acid position that corresponds to amino acid position 392 of the wild type feline IgG.

In some embodiments, the polypeptide comprises:
(i) Leu at the amino acid position that corresponds to amino acid position 428 of the wild type feline IgG and Asp at the amino acid position that corresponds to amino acid position 286 of the wild type feline IgG;
(ii) Leu at the amino acid position that corresponds to amino acid position 428 of the wild type feline IgG and Leu at the amino acid position that corresponds to amino acid position 301 of the wild type feline IgG
(iii) Leu at the amino acid position that corresponds to amino acid position 428 of the wild type feline IgG and Tyr at the amino acid position that corresponds to amino acid position 301 of the wild type feline IgG;
(iv) Leu at the amino acid position that corresponds to amino acid position 428 of the wild type feline IgG and Val at the amino acid position that corresponds to amino acid position 301 of the wild type feline IgG;
(v) Leu at the amino acid position that corresponds to amino acid position 428 of the wild type feline IgG and Asp at the amino acid position that corresponds to amino acid position 309 of the wild type feline IgG;
(vi) Leu at the amino acid position that corresponds to amino acid position 428 of the wild type feline IgG and Leu at the amino acid position that corresponds to amino acid position 377 of the wild type feline IgG;
(vii) Leu at the amino acid position that corresponds to amino acid position 252 of the wild type feline IgG and Tyr at the amino acid position that corresponds to amino acid position 377 of the wild type feline IgG;
(viii) Leu at the amino acid position that corresponds to amino acid position 252 of the wild type feline IgG and Asp at the amino acid position that corresponds to amino acid position 392 of the wild type feline IgG; or
(ix) Leu at the amino acid position that corresponds to amino acid position 252 of the wild type feline IgG and Glu at the amino acid position that corresponds to amino acid position 392 of the wild type feline IgG.

In some embodiments of any one of the preceding aspects, the wild type feline IgG is a feline IgG1a comprising an Fc domain having an amino acid sequence of SEQ ID NO: 1, a feline IgG1b comprising an Fc domain having an amino acid sequence of SEQ ID NO: 2, or a feline IgG2 comprising an Fc domain having an amino acid sequence of SEQ ID NO: 3. In some embodiments, the wild type feline IgG is a feline IgG1a comprising an Fc domain having the amino acid sequence of SEQ ID NO: 1. In some embodiments, the wild type feline IgG is a feline IgG1b comprising an Fc domain having the amino acid sequence of SEQ ID NO: 2. In some embodiments, the wild type feline IgG is a feline IgG2 comprising an Fc domain having the amino acid sequence of SEQ ID NO: 3.

In some embodiments of any one of the preceding aspects, the polypeptide binds to the feline FcRn at a higher level at an acidic pH than at a neutral pH.

In some embodiments, the polypeptide binds to the feline FcRn at a higher level at a pH of 5.5 to 6.0 (e.g., 5.5, 5.6, 5.7, 5.8, 5.9, or 6.0) than at pH 7.4.

In some embodiments of any one of the preceding aspects, the polypeptide further comprises a protein selected from the group consisting of EPO, CTLA4, LFA3, VEGFR1, VEGFR3, IL-1R, IL-4R, GLP-1 receptor agonist, and thrombopoietin binding peptide.

In some embodiments of any one of the preceding aspects, the polypeptide further comprises a binding domain.

In some embodiments, the binding domain comprises an antibody, an antibody fragment, or a ligand-binding portion of a receptor.

In some embodiments, the antibody or the antibody fragment comprises six complementarity determining regions (CDRs) of an immunoglobulin molecule.

In some embodiments, the antibody fragment is selected from the group consisting of Fab, single chain variable fragment (scFv), Fv, Fab′, Fab′-SH, F(ab′)2, nanobody, and diabody.

In some embodiments, the ligand-binding portion of a receptor comprises a ligand binding domain of a feline receptor protein or an extracellular domain of a feline receptor protein.

In some embodiments, the binding domain specifically binds to an antigen selected from the group consisting of NGF, TrKA, ADAMTS, IL-1, IL-2, IL-4, IL-4R, Angiotensin type 1 (AT1) receptor, Angiotensin type 2 (AT2) receptor, IL-5, IL-12, IL-13, IL-31, IL-33, CD3, CD20, CD47, CD52, and complement system complex.

In another aspect, the invention features a pharmaceutical composition comprising (i) any one of the polypeptides disclosed herein, and (ii) a pharmaceutically acceptable excipient.

In another aspect, the invention features a nucleic acid or nucleic acids encoding any one of the polypeptides disclosed herein.

In another aspect, the invention features an expression vector or expression vectors comprising a nucleic acid or nucleic acids encoding any one of the polypeptides disclosed herein.

In another aspect, the invention features a host cell comprising a nucleic acid or nucleic acids encoding any one of the polypeptides disclosed herein, or an expression vector or expression vectors comprising a nucleic acid or nucleic acids encoding any one of the polypeptides disclosed herein.

In another aspect, the invention features a method of making a polypeptide, the method comprising:
(i) providing a nucleic acid or nucleic acids encoding any one of the polypeptides disclosed herein;
(ii) expressing the nucleic acid or nucleic acids in a host cell culture, thereby producing the polypeptide; and, optionally,
(iii) collecting the polypeptide produced in (ii) from the host cell culture.

In another aspect, the invention features a method of treating or preventing a feline disease or disorder in a cat in need thereof, the method comprising administering an effective amount of a composition comprising any one of the polypeptides disclosed herein, or a pharmaceutical composition comprising (i) any one of the polypeptides disclosed herein, and (ii) a pharmaceutically acceptable excipient.

In some embodiments, the feline disease or disorder is an allergic disease, a chronic pain, an acute pain, an inflammatory disease, an autoimmune disease, an endocrine disease, a gastrointestinal disease, a cardiovascular disease, a renal disease, a fertility related disorder, an infectious disease, or a cancer.

In other embodiments, the feline disease or disorder is atopic dermatitis, allergic dermatitis, osteoarthritic pain, arthritis, anemia, or obesity.

In another aspect, the invention features any one of the polypeptides disclosed herein, or a pharmaceutical composition comprising (i) any one of the polypeptides disclosed herein, and (ii) a pharmaceutically acceptable excipient, for use in treatment or prevention of a feline disease or disorder in a cat in need thereof.

In some embodiments, the feline disease or disorder is an allergic disease, a chronic pain, an acute pain, an inflammatory disease, an autoimmune disease, an endocrine disease, a gastrointestinal disease, a cardiovascular disease, a renal disease, a fertility related disorder, an infectious disease, or a cancer.

In other embodiments, the feline disease or disorder is atopic dermatitis, allergic dermatitis, osteoarthritic pain, arthritis, anemia, or obesity.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description and from the claims.

### BRIEF DESCRIPTION OF DRAWINGS

FIG. 1 shows the alignment of the amino acid sequences of the wild-type feline IgG1a Fc region (SEQ ID NO:1) and the wild-type feline IgG1b Fc region (SEQ ID NO:2) with the putative wild-type feline IgG2 Fc region (SEQ ID NO:3). The hinge region lies between the triangles. Arrows indicate the cysteine residues in the hinge region likely involved in disulfide bridges between the two heavy chains (from Strietzel et al., 2014, Vet. Immunol. Immunopathol., 158: 214-223).

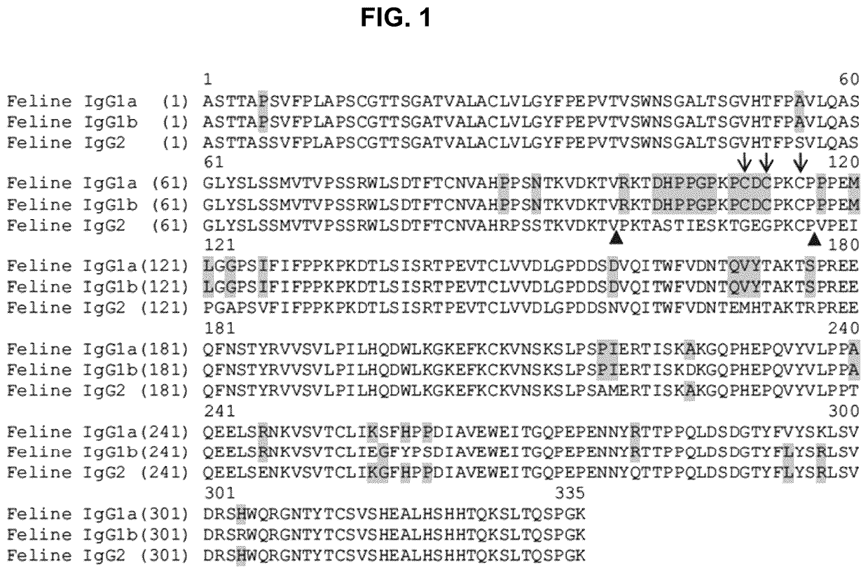

FIG. 2 shows the alignment of the amino acid sequences of the wild-type feline IgG1a Fc (SEQ ID NO: 1) and the human IgG1 Fc region, based on EU numbering.

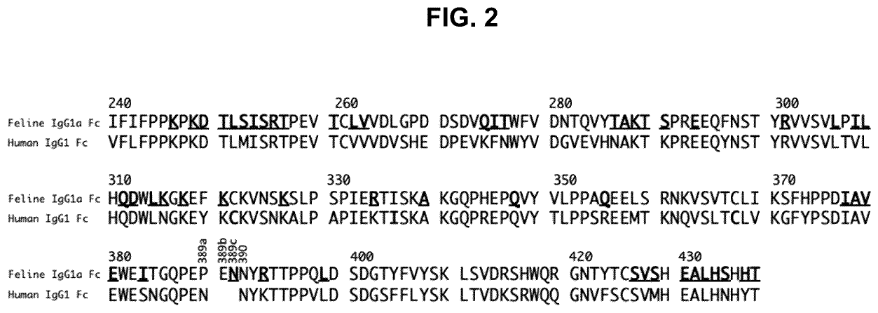

### DETAILED DESCRIPTION

With the increasing use of polypeptides (e.g., antibodies, antigen-binding antibody fragments, ligand-binding domains of receptors, enzymes, ligands, and peptides) as therapeutics for the prevention and treatment of a wide variety of feline diseases, it is important to develop polypeptides with extended half-life, especially for the prevention or treatment of chronic diseases in which a polypeptide must be administered repetitively.

Accordingly, this disclosure features feline immunoglobulin Fc regions or feline FcRn-binding regions thereof comprising mutations that enhance the half-life of a polypeptide or polypeptides comprising these sequences. Also disclosed are polypeptides comprising these domains and methods of their use. These polypeptides can be used for various therapeutic and diagnostic purposes.

For example, this disclosure features polypeptides that have increased binding to feline FcRn, or feline FcRn binding fragments thereof, that are useful in therapeutic polypeptides. For example, provided herein are polypeptides that have increased binding to feline FcRn than control polypeptides (e.g., the wild type counterpart IgG feline Fc regions). In some instances, these polypeptides can, e.g., bind to feline FcRn at a higher level (in other words, with a stronger affinity) at acidic pH (e.g., pH 5.5, pH 6.0, or pH 6.5) than at a neutral pH (e.g., pH 7.0, 7.1, 7.2, 7.3, 7.4, or 7.5). In some instances, these polypeptides bind to feline FcRn at a higher level at pH 5.5 and/or 6.0 than at pH 7.4. This disclosure also relates, in part, to polypeptides that have increased half-life in felines than their wild type counterparts. For example, provided are polypeptides (e.g., binding molecules, such as antibodies, antigen-binding antibody fragments, or ligand-binding portions of receptors) with increased half-life relative to versions of these polypeptides not attached to the Fc regions or feline FcRn binding regions thereof disclosed herein. Also provided are enzyme-Fc region fusions, ligand-Fc region fusions, nanobody-Fc fusions, and peptide-Fc region fusions, wherein the fusions have increased half-life compared with their wild type counterparts. The Fc regions, in addition to having a substitution or substitutions (relative to the wild type feline Fc region) that increase half-life may also include other substitutions that, e.g., increase effector function, decrease effector function, increase binding to Protein A and/or decrease heterogeneity of the polypeptide (e.g., by removing one or more post-translational modifications in the Fc region). The feline Fc region sequences can be from any feline antibody. In some instances, the feline Fc region sequences are from a feline IgG (e.g., IgG1a, IgG1b, IgG2).

Where values are described in terms of ranges, it should be understood that the description includes the disclosure of all possible sub-ranges within such ranges, as well as specific numerical values that fall within such ranges irrespective of whether a specific numerical value or specific sub-range is expressly stated. All numerical designations, e.g., pH, K_{D}, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about” and that a numerical designation may include numerical values that are rounded to the nearest significant figure. It also is to be understood, although not always explicitly stated, that the reagents described herein are merely exemplary and that equivalents of such are known in the art.

Unless otherwise defined, scientific and technical terms used in connection with the present disclosure shall have the meanings that are commonly understood by those of ordinary skill in the art. Further, unless otherwise required by context or expressly indicated, singular terms shall include pluralities and plural terms shall include the singular. For any conflict in definitions between various sources or references, the definition provided herein will control.

It is understood that embodiments of the invention described herein include “comprising,” “consisting,” and “consisting essentially of” aspects and embodiments. As used herein, the singular form “a”, “an,” and “the” includes plural references (e.g., at least one, one or more) unless indicated otherwise. The use of the term “or” herein means “and/or” and is not meant to imply that alternatives are mutually exclusive unless specified otherwise. In the context of a multiple dependent claim, the use of “or” when referring back to other claims refers to those claims in the alternative only.

In this application, the use of “or” means “and/or” unless expressly stated or understood by one skilled in the art. In the context of a multiple dependent claim, the use of “or” refers back to more than one preceding independent or dependent claim.

The term “about,” as used herein when referring to a measurable value such as an amount or concentration and the like, is meant to encompass variations of 20%, 10%, 5%, 1%, 0.5%, or even 0.1% of the specified amount.

As used herein, “percent (%) amino acid sequence identity,” “% identical,” and “homology” with respect to a nucleic acid or polypeptide sequence are defined as the percentage of nucleotides or amino acid residues in a reference sequence that are identical with the nucleotides or amino acid residues in the specific nucleic acid or polypeptide sequence, after aligning the sequences and introducing gaps, if necessary, to achieve the maximum percent sequence identity, and not considering any conservative substitutions as part of the sequence identity. Alignment for purposes of determining percent sequence identity can be achieved in various ways that are within the skill in the art, for instance, using publicly available computer software such as BLAST, BLAST-2, CLUSTAL OMEGA, ALIGN, or MEGALIGN™ (DNASTAR) software. Those skilled in the art can determine appropriate parameters for measuring alignment, including any parameters needed to achieve maximal alignment over the full length of sequences being compared. In some embodiments, a variant has at least 50% sequence identity with the reference nucleic acid molecule or polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added or deleted at the N- or C-terminus of the polypeptide. In some embodiments, a variant has at least 50% sequence identity, at least 60% sequence identity, at least 65% sequence identity, at least 70% sequence identity, at least 75% sequence identity, at least 80% sequence identity, at least 85% sequence identity, at least 90% sequence identity, at least 95% sequence identity, at least 97% sequence identity, at least 98% sequence identity, or at least 99% sequence identity with the sequence of the reference nucleic acid or polypeptide.

The term “amino acid substitution” refers to the replacement of one amino acid in a polypeptide with another amino acid. In some embodiments, an amino acid substitution is a conservative substitution. Amino acid substitutions may be introduced into a polypeptide screened for a desired activity, for example, retained or improved binding to FcRn, retained or improved antigen binding, decreased immunogenicity, improved antibody-dependent cell-mediated cytotoxicity (ADCC) or complement-dependent cytotoxicity (CDC), or enhanced pharmacokinetics.

The term “conservative substitution” as used herein refers to a substitution of one amino acid residue for another amino acid residue that has similar properties such as charge, hydrophobicity, and size. For example, amino acids may be grouped according to common side-chain properties:
(i) hydrophobic: Norleucine (Nle), Met, Ala, Val, Leu, Ile;
(ii) neutral hydrophilic: Cys, Ser, Thr, Asn, Gln;
(iii) acidic: Asp, Glu;
(iv) basic: His, Lys, Arg;
(v) rigid: Gly, Pro;
(vi) aromatic: Trp, Tyr, Phe.

Conservative substitutions will entail exchanging a member of one of these classes with another member of the same class. Non-conservative substitutions will entail exchanging a member of one of these classes with another class. In some embodiments, a conservative amino acid substitution refers to a substitution that results in similar properties or functions as another amino acid substitution. For example, a conservative amino acid substitution of A426Y can be A426F, A426T, or A426W. Additional, nonlimiting examples for conservative amino acid substitutions are shown in Table 1.

TABLE 1

| Original residue | Exemplary conservative substitutions |
|---|---|
| Ala (A) | Gly; Val; Leu; Ile; Ser |
| Arg (R) | Lys; His; Gln; Asn |
| Asn (N) | Gln; His; Asp; Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Nle |
| Leu (L) | Nle; Ile; Val; Met; Ala; Phe |
| Lys (K) | Arg; His; Gln; Asn |
| Met (M) | Leu; Phe; Ile; Tyr |
| Phe (F) | Trp; Leu; Val; Ile; Ala; Tyr; Met |
| Pro (P) | Ala; Gly |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Nle |

The term “affinity” refers to the strength of the sum total of noncovalent interactions between a single binding site of a molecule (e.g., an antibody or a receptor) and its binding partner (e.g., an antigen or a ligand). Unless indicated otherwise, as used herein, “binding affinity” refers to intrinsic binding affinity which reflects a 1:1 interaction between members of a binding pair (e.g., antibody and antigen, receptor and ligand). The affinity of a molecule X for its partner Y can generally be represented by the dissociation constant (K_{D}). Affinity can be measured by common protein-protein interaction tools known in the art, such as, for example, immunoblot, enzyme-linked immunosorbent assay (ELISA), kinetic exclusion assay (KinExA), biolayer interferometry (BLI), or surface plasmon resonance (SPR) devices. Specific illustrative and exemplary embodiments for measuring binding affinity are described in the following.

“Surface plasmon resonance (SPR)” denotes an optical phenomenon that allows for the analysis of real-time biospecific interactions by detection of alterations in protein concentrations within a biosensor matrix, for example, using the BIAcore™ system (BIAcore International AB, a GE Healthcare company, Uppsala, Sweden and Piscataway, N.J.). For further descriptions, see Jonsson et al., 1993, Ann. Biol. Clin. 51: 19-26.

The term “amino acid sequence” refers a sequence of amino acids residues in a peptide or protein. The terms “polypeptide” and “protein” are used interchangeably to refer to a polymer of amino acid residues and are not limited to a minimum length. Such polymers of amino acid residues may contain natural or unnatural amino acid residues, and include, but are not limited to, peptides, oligopeptides, dimers, trimers, and multimers of amino acid residues. Both full-length proteins and fragments thereof are encompassed by the definition. The terms also include postexpression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

The term “antibody” herein is used in the broadest sense and refers to various antibody structures, including but not limited to monoclonal antibodies, polyclonal antibodies, multispecific antibodies (e.g., bispecific antibodies), and antibody fragments (e.g., Fab) so long as they exhibit the desired antigen-binding activity.

The term “antibody fragment” refers to a molecule other than a full-length antibody that comprises a portion of a full-length antibody that binds the antigen to which the full-length antibody binds. In some embodiments, antibody fragments include but are not limited to Fab; single chain variable fragment (e.g., scFv); Fv; Fab′; Fab′-SH; F(ab′)2; nanobody; diabody; and multispecific antibodies formed from antibody fragments.

The terms “full-length antibody” and “whole antibody” are used herein interchangeably to refer to an antibody having a structure substantially similar to a native antibody structure or having heavy chains that contain an Fc region as defined herein.

The terms “nanobody,” “VHH,” “VHH antibody fragment,” and “single domain antibody” as interchangeably used herein denote the variable domain of the single heavy chain of antibodies of the type of those found in Camelidae, which are typically found in natural form to lack light chains. Suitable nanobodies will be familiar to persons skilled in the art, illustrated examples of which include nanobodies of camels, dromedaries, llamas, and alpacas. However, the single domain antibody may be from non-Camelidae sources as well.

The term “binding domain” refers to a part of a compound or a molecule that specifically binds to a target epitope, antigen, ligand, or receptor. Binding domains include but are not limited to antibodies (e.g., monoclonal, polyclonal, recombinant, and chimeric antibodies), antibody fragments or portions thereof (e.g., Fab, scFv, Fv, Fab′, Fab′-SH, F(ab′)₂, nanobody, and diabody), receptors or fragments thereof (e.g., an extracellular domain of a feline receptor protein), ligands, aptamers, and other molecules having an identified binding partner.

The term “chimeric” antibody refers to an antibody in which a portion of the heavy and/or light chain is derived from a particular source or species, while the remainder of the heavy and/or light chain is derived from a different source or species.

The terms “Fc region,” “Fc domain,” and “Fc polypeptide” refers to a C-terminal region of an immunoglobulin heavy chain that contains at least a portion of the constant region. The term “Fc domain of the wild type feline IgG” refers to the native Fc region of a feline antibody. The term “feline IgG Fc region variant” refers to a variant of the Fc region of a feline antibody having a substitution or substitutions relative to the wild type feline Fc region. In some embodiments, the feline Fc region sequences are from a feline IgG (e.g., IgG1a, IgG1b, or IgG2). In some embodiments, the IgG Fc polypeptide comprises the hinge, CH2, and CH3, but does not comprise CH1 or CL. In some embodiments, the IgG Fc polypeptide comprises CH2 and CH3, but does not comprise CH1, the hinge, or CL. In some embodiments, the IgG Fc polypeptide comprises CH1, hinge, CH2, and CH3, with or without CLI. In some embodiments, an Fc polypeptide, such as an IgG Fc polypeptide, lacks one or more C-terminal amino acids, such as 1 to 20, 1 to 15, 1 to 10, 1 to 5, or 1 to 2 amino acids, while retaining biological activity. In some embodiments, the biological activity of an Fc polypeptide is the ability to bind FcRn. Unless otherwise specified herein, numbering of amino acid residues in the Fc region or constant region is according to the EU numbering system, also called the EU index, as described in Kabat et al. Sequences of Proteins of Immunological Interest, 5th Ed. Public Health Service, National Institutes of Health, Bethesda, M D, 1991.

The term “wild type” refers to a non-mutated version of a polypeptide that occurs in nature, or a fragment thereof. A wild type polypeptide may be produced recombinantly. In some embodiments, a wild type IgG Fc domain comprises the amino acid sequence of any one of SEQ ID NOs: 1-3.

The term “disorder” refers to any condition that would benefit from treatment including, but not limited to, chronic and acute disorders or diseases including those pathological conditions which predispose the mammal to the disorder in question.

The term “cancer” refers to or describes the physiological condition in mammals that is typically characterized by unregulated cell growth/proliferation. Examples of cancer include, but are not limited to, myeloma, carcinoma, lymphoma (e.g., Hodgkin's and non-Hodgkin's lymphoma), blastoma, sarcoma (e.g., hemangiosarcoma, osteosarcoma, soft-tissue sarcoma, and histiocytic sarcoma), leukemia, head and neck squamous cell carcinoma, salivary adenocarcinoma, breast cancer, mastocytoma, melanoma, lung cancer (e.g., small-cell lung cancer, non-small cell lung cancer, adenocarcinoma of the lung, and squamous carcinoma of the lung), cancer of the peritoneum, hepatocellular carcinoma, squamous cell carcinoma, meningioma, glioma, gastric cancer, intestinal cancer, colon cancer, colorectal cancer, pancreatic adenocarcinoma, glioblastoma, cervical cancer, endometrial or uterine carcinoma, ovarian cancer, bladder cancer, prostatic carcinoma, kidney or renal cancer, vulval cancer, thyroid cancer, and transitional cell carcinoma.

The term “tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

The term “effector functions” refer to those biological activities attributable to the Fc region of an antibody, which vary with the antibody isotype. Examples of antibody effector functions include: Clq binding and CDC; Fc receptor binding; ADCC; phagocytosis; down regulation of cell surface receptors (e.g., B cell receptor); and B cell activation.

The “effective amount” of a composition, for example, a polypeptide of the present disclosure or a composition (e.g., pharmaceutical composition) thereof, refers to at least the minimum amount required to achieve the desired therapeutic or prophylactic result, such as a measurable improvement or prevention of a particular disorder (e.g., any disorder affecting a feline, e.g., a cell proliferative disorder, e.g., cancer). An effective amount herein may vary according to factors such as the disease state, age, sex, and weight of the animal, and the ability of the antibody to elicit a desired response in the animal. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications, and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

The terms “host cell” and “host cell culture” are used interchangeably and refer to cells into which exogenous nucleic acid has been introduced, including the progeny of such cells. Host cells include bacterial (e.g., E. coli cells) and eukaryotic cells. In some embodiments, host cells include yeast cells (e.g., Pichia (see, e.g., Powers et al., 2001, J Immunol Methods. 251: 123-135), Hanseula, or Saccharomyces). In some embodiments, host cells also include “transformants” and “transformed cells,” which include the primary transformed cell lines (e.g., CHO, 293E, COS, 293T, and HeLa) and progeny derived therefrom without regard to the number of passages. Progeny may not be completely identical in nucleic acid content to a parent cell but may contain mutations. Mutant progeny that has the same function or biological activity as screened or selected for in the originally transformed cell are included herein.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “pharmaceutical composition” refers to a preparation which is in such form as to permit the biological activity of an active ingredient contained therein to be effective, and which contains no additional components which are unacceptably toxic to a subject to which the formulation would be administered.

The term “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, the term “treatment” (and grammatical variations thereof such as “treat” or “treating”) refers to clinical intervention in an attempt to alter the natural course of the individual being treated and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, the polypeptides of the invention are used to delay development of a disease or to slow the progression of a disease.

As used herein, the term “delaying progression” of a disorder or disease means to defer, hinder, slow, retard, stabilize, and/or postpone development of the disease or disorder (e.g., a cell proliferative disorder, e.g., cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late-stage cancer, such as development of metastasis, may be delayed.

The term “epitope” refers to the particular site or sites on an antigen molecule to which an antibody binds. For example, an epitope may be a linear epitope or a conformational epitope.

As used herein, the terms “reduce” and “inhibit” refer to the ability to cause an overall decrease, for example, of 20% or greater, of 50% or greater, or of 75%, 85%, 90%, 95%, or greater, e.g., as compared to a reference or control.

The terms “increase” and “enhance” refer to the ability to cause an overall increase, for example, of 20% or greater, of 50% or greater, or of 75%, 85%, 90%, 95%, or greater, e.g., as compared to a reference or control.

The terms “variable region” and “variable domain” refer to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al., 2007, Kuby Immunology, 6th ed. W.H. Freeman and Co., page 91.) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., 1993, J. Immunol. 150: 880-887; and Clarkson et al., 1991, Nature 352: 624-628.

A “variant” is a polypeptide that differs from a reference polypeptide by single or multiple non-native amino acid substitutions, deletions, and/or additions. In some embodiments, a variant retains at least one biological activity of the reference polypeptide. In some embodiments, a variant has a biological activity that the reference polypeptide substantially lacks. A “feline IgG Fc region variant” comprises an amino acid sequence which differs from that of a wild type feline IgG Fc region by at least one amino acid modification, preferably one or more amino acid substitution(s). Preferably, the feline IgG Fc region variant has at least one amino acid substitution compared to a wild type feline IgG Fc region, e.g., from one to ten amino acid substitutions, and preferably from one to five amino acid substitutions in a wild type feline IgG Fc region. The feline IgG Fc region variant herein will preferably possess at least 80% homology with a wild type feline IgG Fc region, and most preferably at least 90% homology therewith, more preferably at least 95% homology therewith. In some embodiments, the feline IgG Fc region is a feline IgG1a Fc region variant, a feline IgG1b Fc region variant, or a feline IgG2 Fc region variant.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors can direct the expression of nucleic acids to which they are operatively linked. Such vectors are referred to herein as “expression vectors.”

As used herein, “administering” is meant a method of giving a dosage of a compound (e.g., a polypeptide of the present disclosure) or a composition (e.g., a pharmaceutical composition, e.g., a pharmaceutical composition including a polypeptide of the present disclosure) to a subject. The compositions utilized in the methods described herein can be administered, for example, parenterally, intramuscularly, intravenously, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. The administration may be local or systemic. The method of administration can vary depending on various factors (e.g., the compound or composition being administered and the severity of the condition, disease, or disorder being treated).

Administration “in combination with” one or more further therapeutic agents includes simultaneous (concurrent) and consecutive or sequential administration in any order. The term “concurrently” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered with a time separation of no more than about a specified number of minutes. The term “sequentially” is used herein to refer to administration of two or more therapeutic agents where the administration of one or more agent(s) continues after discontinuing the administration of one or more other agent(s), or wherein administration of one or more agent(s) begins before the administration of one or more other agent(s). For example, administration of the two or more therapeutic agents are administered with a time separation of more than about a specified number of minutes. As used herein, “in conjunction with” refers to administration of one treatment modality in addition to another treatment modality. As such, “in conjunction with” refers to administration of one treatment modality before, during or after administration of the other treatment modality to the animal.

### Feline Polypeptides

Cats typically have three IgG heavy chains referred to as IgG1a, IgG1b, and IgG2. These heavy chains represent three different subclasses of cat IgG. The amino acid and DNA sequences for these heavy chains are available from Tang et al., 2001, Vet. Immunol. Immunopathol., 80: 259-270 and the GENBANK database. For example, the amino acid sequence of feline IgG1a heavy chain has GENBANK accession number BAA32229.1, feline IgG1b heavy chain has GENBANK accession number BAA32230.1, and feline IgG2 heavy chain has GENBANK accession number KF811175.1. Feline antibodies also include two types of light chains: kappa and lambda. The DNA and amino acid sequence of these light chains can also be obtained from GENBANK database. For example, the cat kappa light chain amino acid sequence has accession number AF198257.1 and the cat lambda light chain has accession number E07339.1.

### CH2 Region of a Feline Fc Region:

The CH2 region of a feline antibody comprises or consists of amino acids 231 to 340 (according to EU numbering) of a feline IgG antibody. It is to be understood that the CH2 region may include one to six (e.g., 1, 2, 3, 4, 5, or 6) additional amino acids or deletions at their N and/or C-terminus.

The amino acid sequence of the CH2 region of feline IgG1a is provided below:

(SEQ ID NO: 4)
PPEMLGGPSIFIFPPKPKDTLSISRTPEVTCLVVDLGPDDSDV
QITWFVDNTQVYTAKTSPREEQFNSTYRVVSVLPILHQDWLKG
KEFKCKVNSKSLPSPIERTISKAK

The amino acid sequence of the CH2 domain of feline IgG1b is provided below:

(SEQ ID NO: 5)
PPEMLGGPSIFIFPPKPKDTLSISRTPEVTCLVVDLGPDDSDV
QITWFVDNTQVYTAKTSPREEQFNSTYRVVSVLPILHQDWLKG
KEFKCKVNSKSLPSPIERTISKDK

The amino acid sequence of the CH2 domain of feline IgG2 is provided below:

(SEQ ID NO: 6)
VPEIPGAPSVFIFPPKPKDTLSISRTPEVTCLVVDLGPDDSNV
QITWFVDNTEMHTAKTRPREEQFNSTYRVVSVLPILHQDWLKG
KEFKCKVNSKSLPSAMERTISKAK

### CH3 Region of a Feline Fc Region:

The CH3 region of a feline antibody comprises or consists of amino acids 341 to 447 (according to EU numbering) of a feline IgG antibody. It is to be understood that the CH3 region may include one to six (e.g., 1, 2, 3, 4, 5, 6) additional amino acids or deletions at their N and/or C-terminus.

The amino acid sequence of the CH3 domain of feline IgG1a is provided below:

(SEQ ID NO: 7)
GQPHEPQVYVLPPAQEELSRNKVSVTCLIKSFHPPDIAVEWEI
TGQPEPENNYRTTPPQLDSDGTYFVYSKLSVDRSHWQRGNTYT
CSVSHEALHSHHTQKSLTQSPGK

The amino acid sequence of the CH3 domain of feline IgG1b is provided below:

(SEQ ID NO: 8)
GQPHEPQVYVLPPAQEELSRNKVSVTCLIEGFYPSDIAVEWEI
TGQPEPENNYRTTPPQLDSDGTYFLYSRLSVDRSRWQRGNTYT
CSVSHEALHSHHTQKSLTQSPGK

The amino acid sequence of the CH3 domain of feline IgG2 is provided below:

(SEQ ID NO: 9)
GQPHEPQVYVLPPTQEELSENKVSVTCLIKGFHPPDIAVEWEI
TGQPEPENNYQTTPPQLDSDGTYFLYSRLSVDRSHWQRGNTYT
CSVSHEALHSHHTQKSLTQSPGK

### Fc Region of a Feline Fc Region:

The Fc region of a feline IgG antibody comprises or consists of amino acids 231 to 447 (according to EU numbering) of the feline IgG antibody.

The amino acid sequence of the Fc domain of feline IgG1a is provided below:

(SEQ ID NO: 1)
PPEMLGGPSIFIFPPKPKDTLSISRTPEVTCLVVDLGPDDSDV
QITWFVDNTQVYTAKTSPREEQFNSTYRVVSVLPILHQDWLKG
KEFKCKVNSKSLPSPIERTISKAKGQPHEPQVYVLPPAQEELS
RNKVSVTCLIKSFHPPDIAVEWEITGQPEPENNYRTTPPQLDS
DGTYFVYSKLSVDRSHWQRGNTYTCSVSHEALHSHHTQKSLTQ
SPGK

The amino acid sequence of the Fc domain of feline IgG1b is provided below:

(SEQ ID NO: 2)
PPEMLGGPSIFIFPPKPKDTLSISRTPEVTCLVVDLGPDDSDV
QITWFVDNTQVYTAKTSPREEQFNSTYRVVSVLPILHQDWLKG
KEFKCKVNSKSLPSPIERTISKDKGQPHEPQVYVLPPAQEELS
RNKVSVTCLIEGFYPSDIAVEWEITGQPEPENNYRTTPPQLDS
DGTYFLYSRLSVDRSRWQRGNTYTCSVSHEALHSHHTQKSLTQ
SPGK

The amino acid sequence of the Fc domain of feline IgG2 is provided below:

(SEQ ID NO: 3)
VPEIPGAPSVFIFPPKPKDTLSISRTPEVTCLVVDLGPDDSNV
QITWFVDNTEMHTAKTRPREEQFNSTYRVVSVLPILHQDWLKG
KEFKCKVNSKSLPSAMERTISKAKGQPHEPQVYVLPPTQEELS
ENKVSVTCLIKGFHPPDIAVEWEITGQPEPENNYQTTPPQLDS
DGTYFLYSRLSVDRSHWQRGNTYTCSVSHEALHSHHTQKSLTQ
SPGK

Table 2 below compares the amino acid sequences of the CH2 and CH3 domains of human IgG1, feline IgG1a, feline IgG1b, and feline IgG2, based on EU numbering:

TABLE 2

| | | | | |
|---|---|---|---|---|
| | human | feline | feline | feline |
| EU number | IgG1 | IgG1a | IgG1b | IgG2 |

CH2 Domain

| | | | | |
|---|---|---|---|---|
| 231 | A | P | P | V |
| 232 | P | P | P | P |
| 233 | E | E | E | E |
| 234 | L | M | M | I |
| 235 | L | L | L | P |
| 236 | G | G | G | G |
| 237 | G | G | G | A |
| 238 | P | P | P | P |
| 239 | S | S | S | S |
| 240 | V | I | I | V |
| 241 | F | F | F | F |
| 242 | L | I | I | I |
| 243 | F | F | F | F |
| 244 | P | P | P | P |
| 245 | P | P | P | P |
| 246 | K | K | K | K |
| 247 | P | P | P | P |
| 248 | K | K | K | K |
| 249 | D | D | D | D |
| 250 | T | T | T | T |
| 251 | L | L | L | L |
| 252 | M | S | S | S |
| 253 | I | I | I | O |
| 254 | S | S | S | S |
| 255 | R | R | R | R |
| 256 | T | T | T | T |
| 257 | P | P | P | P |
| 258 | E | E | E | E |
| 259 | V | V | V | V |
| 260 | T | T | T | T |
| 261 | C | C | C | C |
| 262 | V | L | L | L |
| 263 | V | V | V | V |
| 264 | V | V | V | V |
| 265 | D | D | D | D |
| 266 | V | L | L | L |
| 267 | S | G | G | G |
| 268 | H | P | P | P |
| 269 | E | D | D | D |
| 270 | D | D | D | D |
| 271 | P | S | S | S |
| 272 | E | D | D | N |
| 273 | V | V | V | V |
| 274 | K | Q | Q | Q |
| 275 | F | I | I | I |
| 276 | N | T | T | T |
| 277 | W | W | W | W |
| 278 | Y | F | F | F |
| 279 | V | V | V | V |
| 280 | D | D | D | D |
| 281 | G | N | N | N |
| 282 | V | T | T | T |
| 283 | E | Q | Q | E |
| 284 | V | V | V | M |
| 285 | H | Y | Y | H |
| 286 | N | T | T | T |
| 287 | A | A | A | A |
| 288 | K | K | K | K |
| 289 | T | T | T | T |
| 290 | K | S | S | R |
| 291 | P | P | P | P |
| 292 | R | R | R | R |
| 293 | E | E | E | E |
| 294 | E | E | E | E |
| 295 | Q | Q | Q | Q |
| 296 | Y | F | F | F |
| 297 | N | N | N | N |
| 298 | S | S | S | S |
| 299 | T | T | T | T |
| 300 | Y | Y | Y | Y |
| 301 | R | R | R | R |
| 302 | V | V | V | V |
| 303 | V | V | V | V |
| 304 | S | S | S | S |
| 305 | V | V | V | V |
| 306 | L | L | L | L |
| 307 | T | P | P | P |
| 308 | V | I | I | I |
| 309 | L | L | L | L |
| 310 | H | H | H | H |
| 311 | Q | Q | Q | Q |
| 312 | D | D | D | D |
| 313 | W | W | W | W |
| 314 | L | L | L | L |
| 315 | N | K | K | K |
| 316 | G | G | G | G |
| 317 | K | K | K | K |
| 318 | E | E | E | E |
| 319 | Y | F | F | F |
| 320 | K | K | K | K |
| 321 | C | C | C | C |
| 322 | K | K | K | K |
| 323 | V | V | V | V |
| 324 | S | N | N | N |
| 325 | N | S | S | S |
| 326 | K | K | K | K |
| 327 | A | S | S | S |
| 328 | L | L | L | L |
| 329 | P | P | P | P |
| 330 | A | S | S | S |
| 331 | P | P | P | A |
| 332 | I | I | I | M |
| 333 | E | E | E | E |
| 334 | K | R | R | R |
| 335 | T | T | T | T |
| 336 | I | I | I | I |
| 337 | S | S | S | S |
| 338 | K | K | K | K |
| 339 | A | A | D | A |
| 340 | K | K | K | K |

CH3 Domain

| | | | | |
|---|---|---|---|---|
| 341 | G | G | G | G |
| 342 | Q | Q | Q | Q |
| 343 | P | P | P | P |
| 344 | R | H | H | H |
| 345 | E | E | E | E |
| 346 | P | P | P | P |
| 347 | Q | Q | Q | Q |
| 348 | V | V | V | V |
| 349 | Y | Y | Y | Y |
| 350 | T | V | V | V |
| 351 | L | L | L | L |
| 352 | P | P | P | P |
| 353 | P | P | P | P |
| 354 | S | A | A | T |
| 355 | R | Q | Q | Q |
| 356 | D | E | E | E |
| 357 | E | E | E | E |
| 358 | L | L | L | L |
| 359 | T | S | S | S |
| 360 | K | R | R | E |
| 361 | N | N | N | N |
| 362 | Q | K | K | K |
| 363 | V | V | V | V |
| 364 | S | S | S | S |
| 365 | L | V | V | V |
| 366 | T | T | T | T |
| 367 | C | C | C | C |
| 368 | L | L | L | L |
| 369 | V | I | I | I |
| 370 | K | K | E | K |
| 371 | G | S | G | G |
| 372 | F | F | F | F |
| 373 | Y | H | Y | H |
| 374 | P | P | P | P |
| 375 | S | P | S | P |
| 376 | D | D | D | D |
| 377 | I | I | I | I |
| 378 | A | A | A | A |
| 379 | V | V | V | V |
| 380 | E | E | E | E |
| 381 | W | W | W | W |
| 382 | E | E | E | E |
| 383 | S | I | I | I |
| 384 | N | T | T | T |
| 385 | G | G | G | G |
| 386 | Q | Q | Q | Q |
| 387 | P | P | P | P |
| 388 | E | E | E | E |
| 389a | N | P | P | P |
| 389b | | E | E | E |
| 389c | | N | N | N |
| 390 | N | N | N | N |
| 391 | Y | Y | Y | Y |
| 392 | K | R | R | Q |
| 393 | T | T | T | T |
| 394 | T | T | T | T |
| 395 | P | P | P | P |
| 396 | P | P | P | P |
| 397 | V | Q | Q | Q |
| 398 | L | L | L | L |
| 399 | D | D | D | D |
| 400 | S | S | S | S |
| 401 | D | D | D | D |
| 402 | G | G | G | G |
| 403 | S | T | T | T |
| 404 | F | Y | Y | Y |
| 405 | F | F | F | F |
| 406 | L | V | L | L |
| 407 | Y | Y | Y | Y |
| 408 | S | S | S | S |
| 409 | K | K | R | R |
| 410 | L | L | L | L |
| 411 | T | S | S | S |
| 412 | V | V | V | V |
| 413 | D | D | D | D |
| 414 | K | R | R | R |
| 415 | S | S | S | S |
| 416 | R | H | R | H |
| 417 | W | W | W | W |
| 418 | Q | Q | Q | Q |
| 419 | Q | R | R | R |
| 420 | G | G | G | G |
| 421 | N | N | N | N |
| 422 | V | T | T | T |
| 423 | F | Y | Y | Y |
| 424 | S | T | T | T |
| 425 | C | C | C | C |
| 426 | S | S | S | S |
| 427 | V | V | V | V |
| 428 | M | S | S | S |
| 429 | H | H | H | H |
| 430 | E | E | E | E |
| 431 | A | A | A | A |
| 432 | L | L | L | L |
| 433 | H | H | H | H |
| 434 | N | S | S | S |
| 435 | H | H | H | H |
| 436 | Y | H | H | H |
| 437 | T | T | T | T |
| 438 | Q | Q | Q | Q |
| 439 | K | K | K | K |
| 440 | S | S | S | S |
| 441 | L | L | L | L |
| 442 | S | T | T | T |
| 443 | L | Q | Q | Q |
| 444 | S | S | S | S |
| 445 | P | P | P | P |
| 446 | G | G | G | G |
| 447 | K | K | K | K |

Substitutions in Feline IgG Fc that Improve Half-Life

Increased serum persistence is a beneficial property for therapeutic polypeptides. This disclosure features substitutions in wild type feline IgG1a, IgG1b, and IgG2 Fc regions that enhance the half-life of a polypeptide or polypeptides comprising these Fc regions in a cat relative to a control polypeptide or control polypeptides, wherein the control polypeptide or control polypeptides are identical to the polypeptide or polypeptides except for having the corresponding wild type feline IgG Fc region in place of the IgG Fc region variant. The substitutions to increase half-life may be made in one or more of a feline CH2 region, a feline CH3 region, or in the context of a feline Fc (e.g., a CH2+CH3) region.

The present disclosure provides a polypeptide comprising a feline IgG Fc region variant, wherein the feline IgG Fc region variant comprises (i) an amino acid substitution (e.g., Tyr) at a position that corresponds to amino acid position 252 of a wild type feline IgG, and (ii) at least one amino acid substitution at a position selected from the group consisting of:
(i) a position that corresponds to amino acid position 286 of a wild type feline IgG;
(ii) a position that corresponds to amino acid position 301 of a wild type feline IgG;
(iii) a position that corresponds to amino acid position 309 of a wild type feline IgG;
(iv) a position that corresponds to amino acid position 377 of a wild type feline IgG; and
(v) a position that corresponds to amino acid position 392 of a wild type feline IgG;
wherein the amino acid positions are based on EU numbering, and wherein the polypeptide has increased binding affinity to feline FcRn compared to an Fc domain of the wild type feline IgG. In some examples, the amino acid substitution at the position that corresponds to amino acid position 252 of a wild type feline IgG is a conservative amino acid substitution of Tyr. In some examples, the amino acid substitution at the position that corresponds to amino acid position 309 of a wild type feline IgG is a conservative amino acid substitution of Asp or Val.

For example, the present disclosure provides a polypeptide comprising a feline IgG Fc region variant, wherein the feline IgG Fc region variant comprises (i) Tyr at a position that corresponds to amino acid position 252 of a wild type feline IgG, and (ii) at least one amino acid substitution at a position selected from the group consisting of:
(i) a position that corresponds to amino acid position 286 of a wild type feline IgG;
(ii) a position that corresponds to amino acid position 301 of a wild type feline IgG;
(iii) a position that corresponds to amino acid position 309 of a wild type feline IgG, wherein the amino acid substitution is Asp or Val;
(iv) a position that corresponds to amino acid position 377 of a wild type feline IgG; and
(v) a position that corresponds to amino acid position 392 of a wild type feline IgG;
wherein the amino acid positions are based on EU numbering, and wherein the polypeptide has increased binding affinity to feline FcRn compared to an Fc domain of the wild type feline IgG. In some examples, the IgG Fc region variant comprises Asp at a position that corresponds to amino acid position 309 of a wild type feline IgG. In some examples, the IgG Fc region variant comprises Val at a position that corresponds to amino acid position 309 of a wild type feline IgG.

In some embodiments, the polypeptide comprises Asp or Glu at the amino acid position that corresponds to amino acid position 286 of the wild type feline IgG. In some embodiments, the polypeptide comprises Asp at the amino acid position that corresponds to amino acid position 286 of the wild type feline IgG. In some embodiments, the polypeptide comprises Glu at the amino acid position that corresponds to amino acid position 286 of the wild type feline IgG. In some embodiments, the amino acid substitution at the position that corresponds to amino acid position 286 of a wild type feline IgG is a conservative amino acid substitution of Asp or Glu.

In some embodiments, the polypeptide comprises Leu, Tyr, or Val at the amino acid position that corresponds to amino acid position 301 of the wild type feline IgG. In some embodiments, the polypeptide comprises Leu at the amino acid position that corresponds to amino acid position 301 of the wild type feline IgG. In some embodiments, the polypeptide comprises Tyr at the amino acid position that corresponds to amino acid position 301 of the wild type feline IgG. In some embodiments, the polypeptide comprises Val at the amino acid position that corresponds to amino acid position 301 of the wild type feline IgG. In some embodiments, the amino acid substitution at the position that corresponds to amino acid position 301 of a wild type feline IgG is a conservative amino acid substitution of Leu, Tyr, or Val.

In some embodiments, the polypeptide comprises Leu or Tyr at the amino acid position that corresponds to amino acid position 377 of the wild type feline IgG. In some embodiments, the polypeptide comprises Leu at the amino acid position that corresponds to amino acid position 377 of the wild type feline IgG. In some embodiments, the polypeptide comprises Tyr at the amino acid position that corresponds to amino acid position 377 of the wild type feline IgG. In some embodiments, the amino acid substitution at the position that corresponds to amino acid position 377 of a wild type feline IgG is a conservative amino acid substitution of Leu or Tyr.

In some embodiments, the polypeptide comprises Asp or Glu at the amino acid position that corresponds to amino acid position 392 of the wild type feline IgG. In some embodiments, the polypeptide comprises Asp at the amino acid position that corresponds to amino acid position 392 of the wild type feline IgG. In some embodiments, the polypeptide comprises Glu at the amino acid position that corresponds to amino acid position 392 of the wild type feline IgG. In some embodiments, the amino acid substitution at the position that corresponds to amino acid position 392 of a wild type feline IgG is a conservative amino acid substitution of Asp or Glu.

In some embodiments, the polypeptide comprises:
(i) Tyr at the amino acid position that corresponds to amino acid position 252 of the wild type feline IgG and Asp at the amino acid position that corresponds to amino acid position 286 of the wild type feline IgG;
(ii) Tyr at the amino acid position that corresponds to amino acid position 252 of the wild type feline IgG and Glu at the amino acid position that corresponds to amino acid position 286 of the wild type feline IgG;
(iii) Tyr at the amino acid position that corresponds to amino acid position 252 of the wild type feline IgG and Leu at the amino acid position that corresponds to amino acid position 301 of the wild type feline IgG;
(iv) Tyr at the amino acid position that corresponds to amino acid position 252 of the wild type feline IgG and Tyr at the amino acid position that corresponds to amino acid position 301 of the wild type feline IgG;
(v) Tyr at the amino acid position that corresponds to amino acid position 252 of the wild type feline IgG and Val at the amino acid position that corresponds to amino acid position 301 of the wild type feline IgG;
(vi) Tyr at the amino acid position that corresponds to amino acid position 252 of the wild type feline IgG and Asp at the amino acid position that corresponds to amino acid position 309 of the wild type feline IgG;
(vii) Tyr at the amino acid position that corresponds to amino acid position 252 of the wild type feline IgG and Val at the amino acid position that corresponds to amino acid position 309 of the wild type feline IgG;
(viii) Tyr at the amino acid position that corresponds to amino acid position 252 of the wild type feline IgG and Leu at the amino acid position that corresponds to amino acid position 377 of the wild type feline IgG;
(ix) Tyr at the amino acid position that corresponds to amino acid position 252 of the wild type feline IgG and Tyr at the amino acid position that corresponds to amino acid position 377 of the wild type feline IgG;
(x) Tyr at the amino acid position that corresponds to amino acid position 252 of the wild type feline IgG and Asp at the amino acid position that corresponds to amino acid position 392 of the wild type feline IgG; or
(xi) Tyr at the amino acid position that corresponds to amino acid position 252 of the wild type feline IgG and Glu at the amino acid position that corresponds to amino acid position 392 of the wild type feline IgG.

In another aspect, the invention features a polypeptide comprising a feline IgG Fc region variant, wherein the feline IgG Fc region variant comprises (i) an amino acid substitution (e.g., Met) at a position that corresponds to amino acid position 252 of a wild type feline IgG, and (ii) at least one amino acid substitution at a position selected from the group consisting of:
(i) a position that corresponds to amino acid position 286 of a wild type feline IgG;
(ii) a position that corresponds to amino acid position 301 of a wild type feline IgG;
(iii) a position that corresponds to amino acid position 309 of a wild type feline IgG;
(iv) a position that corresponds to amino acid position 311 of a wild type feline IgG;
(v) a position that corresponds to amino acid position 377 of a wild type feline IgG; and
(vi) a position that corresponds to amino acid position 392 of a wild type feline IgG;
wherein the amino acid positions are based on EU numbering, and wherein the polypeptide has increased binding affinity to feline FcRn compared to an Fc domain of the wild type feline IgG. In some examples, the amino acid substitution at the position that corresponds to amino acid position 252 of a wild type feline IgG is a conservative amino acid substitution of Met. In some examples, the amino acid substitution at the position that corresponds to amino acid position 309 of a wild type feline IgG is a conservative amino acid substitution of Asp or Val. In some examples, the amino acid substitution at the position that corresponds to amino acid position 311 of a wild type feline IgG is a conservative amino acid substitution of Val.

For example, the invention features a polypeptide comprising a feline IgG Fc region variant, wherein the feline IgG Fc region variant comprises (i) Met at a position that corresponds to amino acid position 252 of a wild type feline IgG, and (ii) at least one amino acid substitution at a position selected from the group consisting of:
(i) a position that corresponds to amino acid position 286 of a wild type feline IgG;
(ii) a position that corresponds to amino acid position 301 of a wild type feline IgG;
(iii) a position that corresponds to amino acid position 309 of a wild type feline IgG, wherein the amino acid substitution is Asp or Val;
(iv) a position that corresponds to amino acid position 311 of a wild type feline IgG, wherein the amino acid substitution is Val;
(v) a position that corresponds to amino acid position 377 of a wild type feline IgG; and
(vi) a position that corresponds to amino acid position 392 of a wild type feline IgG;
wherein the amino acid positions are based on EU numbering, and wherein the polypeptide has increased binding affinity to feline FcRn compared to an Fc domain of the wild type feline IgG. In some examples, the IgG Fc region variant comprises Asp at a position that corresponds to amino acid position 309 of a wild type feline IgG. In some examples, the IgG Fc region variant comprises Val at a position that corresponds to amino acid position 309 of a wild type feline IgG.

In some embodiments, the polypeptide comprises Asp or Glu at the amino acid position that corresponds to amino acid position 286 of the wild type feline IgG. In some embodiments, the polypeptide comprises Asp at the amino acid position that corresponds to amino acid position 286 of the wild type feline IgG. In some embodiments, the polypeptide comprises Glu at the amino acid position that corresponds to amino acid position 286 of the wild type feline IgG. In some embodiments, the amino acid substitution at the position that corresponds to amino acid position 286 of a wild type feline IgG is a conservative amino acid substitution of Asp or Glu.

In some embodiments, the polypeptide comprises Leu, Tyr, or Val at the amino acid position that corresponds to amino acid position 301 of the wild type feline IgG. In some embodiments, the polypeptide comprises Leu at the amino acid position that corresponds to amino acid position 301 of the wild type feline IgG. In some embodiments, the polypeptide comprises Tyr at the amino acid position that corresponds to amino acid position 301 of the wild type feline IgG. In some embodiments, the polypeptide comprises Val at the amino acid position that corresponds to amino acid position 301 of the wild type feline IgG. In some embodiments, the amino acid substitution at the position that corresponds to amino acid position 301 of a wild type feline IgG is a conservative amino acid substitution of Leu, Tyr, or Val.

In some embodiments, the polypeptide comprises Val at the amino acid position that corresponds to amino acid position 311 of the wild type feline IgG. In some embodiments, the amino acid substitution at the position that corresponds to amino acid position 311 of a wild type feline IgG is a conservative amino acid substitution of Val.

In some embodiments, the polypeptide comprises Leu or Tyr at the amino acid position that corresponds to amino acid position 377 of the wild type feline IgG. In some embodiments, the polypeptide comprises Leu at the amino acid position that corresponds to amino acid position 377 of the wild type feline IgG. In some embodiments, the polypeptide comprises Tyr at the amino acid position that corresponds to amino acid position 377 of the wild type feline IgG. In some embodiments, the amino acid substitution at the position that corresponds to amino acid position 377 of a wild type feline IgG is a conservative amino acid substitution of Leu or Tyr.

In some embodiments, the polypeptide comprises Asp or Glu at the amino acid position that corresponds to amino acid position 392 of the wild type feline IgG. In some embodiments, the polypeptide comprises Asp at the amino acid position that corresponds to amino acid position 392 of the wild type feline IgG. In some embodiments, the polypeptide comprises Glu at the amino acid position that corresponds to amino acid position 392 of the wild type feline IgG. In some embodiments, the amino acid substitution at the position that corresponds to amino acid position 392 of a wild type feline IgG is a conservative amino acid substitution of Asp or Glu.

In some embodiments, the polypeptide comprises:
(i) Met at the amino acid position that corresponds to amino acid position 252 of the wild type feline IgG and Asp at the amino acid position that corresponds to amino acid position 286 of the wild type feline IgG;
(ii) Met at the amino acid position that corresponds to amino acid position 252 of the wild type feline IgG and Glu at the amino acid position that corresponds to amino acid position 286 of the wild type feline IgG;
(iii) Met at the amino acid position that corresponds to amino acid position 252 of the wild type feline IgG and Leu at the amino acid position that corresponds to amino acid position 301 of the wild type feline IgG
(iv) Met at the amino acid position that corresponds to amino acid position 252 of the wild type feline IgG and Tyr at the amino acid position that corresponds to amino acid position 301 of the wild type feline IgG;
(v) Met at the amino acid position that corresponds to amino acid position 252 of the wild type feline IgG and Val at the amino acid position that corresponds to amino acid position 301 of the wild type feline IgG;
(vi) Met at the amino acid position that corresponds to amino acid position 252 of the wild type feline IgG and Asp at the amino acid position that corresponds to amino acid position 309 of the wild type feline IgG;
(vii) Met at the amino acid position that corresponds to amino acid position 252 of the wild type feline IgG and Val at the amino acid position that corresponds to amino acid position 309 of the wild type feline IgG;
(viii) Met at the amino acid position that corresponds to amino acid position 252 of the wild type feline IgG and Val at the amino acid position that corresponds to amino acid position 311 of the wild type feline IgG;
(ix) Met at the amino acid position that corresponds to amino acid position 252 of the wild type feline IgG and Leu at the amino acid position that corresponds to amino acid position 377 of the wild type feline IgG;
(x) Met at the amino acid position that corresponds to amino acid position 252 of the wild type feline IgG and Tyr at the amino acid position that corresponds to amino acid position 377 of the wild type feline IgG;
(xi) Met at the amino acid position that corresponds to amino acid position 252 of the wild type feline IgG and Asp at the amino acid position that corresponds to amino acid position 392 of the wild type feline IgG; or
(xii) Met at the amino acid position that corresponds to amino acid position 252 of the wild type feline IgG and Glu at the amino acid position that corresponds to amino acid position 392 of the wild type feline IgG.

In another aspect, the invention features a polypeptide comprising a feline IgG Fc region variant, wherein the feline IgG Fc region variant comprises (i) an amino acid substitution (e.g., Met) at a position that corresponds to amino acid position 428 of a wild type feline IgG, and (ii) at least one amino acid substitution at a position selected from the group consisting of:
(i) a position that corresponds to amino acid position 286 of a wild type feline IgG;
(ii) a position that corresponds to amino acid position 301 of a wild type feline IgG;
(iii) a position that corresponds to amino acid position 309 of a wild type feline IgG;
(iv) a position that corresponds to amino acid position 311 of a wild type feline IgG;
(v) a position that corresponds to amino acid position 377 of a wild type feline IgG; and
(vi) a position that corresponds to amino acid position 392 of a wild type feline IgG;
wherein the amino acid positions are based on EU numbering, and wherein the polypeptide has increased binding affinity to feline FcRn compared to an Fc domain of the wild type feline IgG. In some examples, the amino acid substitution at the position that corresponds to amino acid position 311 of a wild type feline IgG is a conservative amino acid substitution of Val. In some examples, the amino acid substitution at the position that corresponds to amino acid position 428 of a wild type feline IgG is a conservative amino acid substitution of Met.

For example, the invention features a polypeptide comprising a feline IgG Fc region variant, wherein the feline IgG Fc region variant comprises (i) Met at a position that corresponds to amino acid position 428 of a wild type feline IgG, and (ii) at least one amino acid substitution at a position selected from the group consisting of:
(i) a position that corresponds to amino acid position 286 of a wild type feline IgG;
(ii) a position that corresponds to amino acid position 301 of a wild type feline IgG;
(iii) a position that corresponds to amino acid position 309 of a wild type feline IgG;
(iv) a position that corresponds to amino acid position 311 of a wild type feline IgG, wherein the amino acid substitution is Val;
(v) a position that corresponds to amino acid position 377 of a wild type feline IgG; and
(vi) a position that corresponds to amino acid position 392 of a wild type feline IgG;
wherein the amino acid positions are based on EU numbering, and wherein the polypeptide has increased binding affinity to feline FcRn compared to an Fc domain of the wild type feline IgG.

In some embodiments, the polypeptide comprises Asp or Glu at the amino acid position that corresponds to amino acid position 286 of the wild type feline IgG. In some embodiments, the polypeptide comprises Asp at the amino acid position that corresponds to amino acid position 286 of the wild type feline IgG. In some embodiments, the polypeptide comprises Glu at the amino acid position that corresponds to amino acid position 286 of the wild type feline IgG. In some embodiments, the amino acid substitution at the position that corresponds to amino acid position 286 of a wild type feline IgG is a conservative amino acid substitution of Asp or Glu.

In some embodiments, the polypeptide comprises Leu, Tyr, or Val at the amino acid position that corresponds to amino acid position 301 of the wild type feline IgG. In some embodiments, the polypeptide comprises Leu at the amino acid position that corresponds to amino acid position 301 of the wild type feline IgG. In some embodiments, the polypeptide comprises Tyr at the amino acid position that corresponds to amino acid position 301 of the wild type feline IgG. In some embodiments, the polypeptide comprises Val at the amino acid position that corresponds to amino acid position 301 of the wild type feline IgG. In some embodiments, the amino acid substitution at the position that corresponds to amino acid position 301 of a wild type feline IgG is a conservative amino acid substitution of Leu, Tyr, or Val.

In some embodiments, the polypeptide comprises Asp, Glu, or Val at the amino acid position that corresponds to amino acid position 309 of the wild type feline IgG. In some embodiments, the polypeptide comprises Asp at the amino acid position that corresponds to amino acid position 309 of the wild type feline IgG. In some embodiments, the polypeptide comprises Glu at the amino acid position that corresponds to amino acid position 309 of the wild type feline IgG. In some embodiments, the polypeptide comprises Val at the amino acid position that corresponds to amino acid position 309 of the wild type feline IgG. In some embodiments, the amino acid substitution at the position that corresponds to amino acid position 309 of a wild type feline IgG is a conservative amino acid substitution of Asp, Glu, or Val.

In some embodiments, the polypeptide comprises Val at the amino acid position that corresponds to amino acid position 311 of the wild type feline IgG. In some embodiments, the amino acid substitution at the position that corresponds to amino acid position 311 of a wild type feline IgG is a conservative amino acid substitution of Val.

In some embodiments, the polypeptide comprises Leu or Tyr at the amino acid position that corresponds to amino acid position 377 of the wild type feline IgG. In some embodiments, the polypeptide comprises Leu at the amino acid position that corresponds to amino acid position 377 of the wild type feline IgG. In some embodiments, the polypeptide comprises Tyr at the amino acid position that corresponds to amino acid position 377 of the wild type feline IgG. In some embodiments, the amino acid substitution at the position that corresponds to amino acid position 377 of a wild type feline IgG is a conservative amino acid substitution of Leu or Tyr.

In some embodiments, the polypeptide comprises Asp or Glu at the amino acid position that corresponds to amino acid position 392 of the wild type feline IgG. In some embodiments, the polypeptide comprises Asp at the amino acid position that corresponds to amino acid position 392 of the wild type feline IgG. In some embodiments, the polypeptide comprises Glu at the amino acid position that corresponds to amino acid position 392 of the wild type feline IgG. In some embodiments, the amino acid substitution at the position that corresponds to amino acid position 392 of a wild type feline IgG is a conservative amino acid substitution of Asp or Glu.

In some embodiments, the polypeptide comprises:
(i) Met at the amino acid position that corresponds to amino acid position 428 of the wild type feline IgG and Asp at the amino acid position that corresponds to amino acid position 286 of the wild type feline IgG;
(ii) Met at the amino acid position that corresponds to amino acid position 428 of the wild type feline IgG and Glu at the amino acid position that corresponds to amino acid position 286 of the wild type feline IgG;
(iii) Met at the amino acid position that corresponds to amino acid position 428 of the wild type feline IgG and Leu at the amino acid position that corresponds to amino acid position 301 of the wild type feline IgG
(iv) Met at the amino acid position that corresponds to amino acid position 428 of the wild type feline IgG and Tyr at the amino acid position that corresponds to amino acid position 301 of the wild type feline IgG;
(v) Met at the amino acid position that corresponds to amino acid position 428 of the wild type feline IgG and Val at the amino acid position that corresponds to amino acid position 301 of the wild type feline IgG;
(vi) Met at the amino acid position that corresponds to amino acid position 428 of the wild type feline IgG and Asp at the amino acid position that corresponds to amino acid position 309 of the wild type feline IgG;
(vii) Met at the amino acid position that corresponds to amino acid position 428 of the wild type feline IgG and Glu at the amino acid position that corresponds to amino acid position 309 of the wild type feline IgG;
(viii) Met at the amino acid position that corresponds to amino acid position 428 of the wild type feline IgG and Val at the amino acid position that corresponds to amino acid position 309 of the wild type feline IgG;
(ix) Met at the amino acid position that corresponds to amino acid position 428 of the wild type feline IgG and Val at the amino acid position that corresponds to amino acid position 311 of the wild type feline IgG;
(x) Met at the amino acid position that corresponds to amino acid position 428 of the wild type feline IgG and Leu at the amino acid position that corresponds to amino acid position 377 of the wild type feline IgG;
(xi) Met at the amino acid position that corresponds to amino acid position 252 of the wild type feline IgG and Tyr at the amino acid position that corresponds to amino acid position 377 of the wild type feline IgG;
(xii) Met at the amino acid position that corresponds to amino acid position 252 of the wild type feline IgG and Asp at the amino acid position that corresponds to amino acid position 392 of the wild type feline IgG; or
(xiii) Met at the amino acid position that corresponds to amino acid position 252 of the wild type feline IgG and Glu at the amino acid position that corresponds to amino acid position 392 of the wild type feline IgG.

In another aspect, the invention features a polypeptide comprising a feline IgG Fc region variant, wherein the feline IgG Fc region variant comprises (i) an amino acid substitution (e.g., Leu) at a position that corresponds to amino acid position 428 of a wild type feline IgG, and (ii) at least one amino acid substitution at a position selected from the group consisting of:
(i) a position that corresponds to amino acid position 286 of a wild type feline IgG;
(ii) a position that corresponds to amino acid position 301 of a wild type feline IgG;
(iii) a position that corresponds to amino acid position 309 of a wild type feline IgG;
(iv) a position that corresponds to amino acid position 377 of a wild type feline IgG; and
(v) a position that corresponds to amino acid position 392 of a wild type feline IgG;
wherein the amino acid positions are based on EU numbering, and wherein the polypeptide has increased binding affinity to feline FcRn compared to an Fc domain of the wild type feline IgG. In some examples, the amino acid substitution at the position that corresponds to amino acid position 309 of a wild type feline IgG is a conservative amino acid substitution of Asp. In some examples, the amino acid substitution at the position that corresponds to amino acid position 428 of a wild type feline IgG is a conservative amino acid substitution of Leu.

For example, the invention features a polypeptide comprising a feline IgG Fc region variant, wherein the feline IgG Fc region variant comprises (i) Leu at a position that corresponds to amino acid position 428 of a wild type feline IgG, and (ii) at least one amino acid substitution at a position selected from the group consisting of:
(i) a position that corresponds to amino acid position 286 of a wild type feline IgG;
(ii) a position that corresponds to amino acid position 301 of a wild type feline IgG;
(iii) a position that corresponds to amino acid position 309 of a wild type feline IgG, wherein the amino acid substitution is Asp;
(iv) a position that corresponds to amino acid position 377 of a wild type feline IgG; and
(v) a position that corresponds to amino acid position 392 of a wild type feline IgG;
wherein the amino acid positions are based on EU numbering, and wherein the polypeptide has increased binding affinity to feline FcRn compared to an Fc domain of the wild type feline IgG.

In some embodiments, the polypeptide comprises Asp at the amino acid position that corresponds to amino acid position 286 of the wild type feline IgG. In some embodiments, the amino acid substitution at the position that corresponds to amino acid position 286 of a wild type feline IgG is a conservative amino acid substitution of Asp.

In some embodiments, the polypeptide comprises Leu, Tyr, or Val at the amino acid position that corresponds to amino acid position 301 of the wild type feline IgG. In some embodiments, the polypeptide comprises Leu at the amino acid position that corresponds to amino acid position 301 of the wild type feline IgG. In some embodiments, the polypeptide comprises Tyr at the amino acid position that corresponds to amino acid position 301 of the wild type feline IgG. In some embodiments, the polypeptide comprises Val at the amino acid position that corresponds to amino acid position 301 of the wild type feline IgG. In some embodiments, the amino acid substitution at the position that corresponds to amino acid position 301 of a wild type feline IgG is a conservative amino acid substitution of Leu, Tyr, or Val.

In some embodiments, the polypeptide comprises Asp at the amino acid position that corresponds to amino acid position 309 of the wild type feline IgG. In some embodiments, the amino acid substitution at the position that corresponds to amino acid position 309 of a wild type feline IgG is a conservative amino acid substitution of Asp.

In some embodiments, the polypeptide comprises Leu or Tyr at the amino acid position that corresponds to amino acid position 377 of the wild type feline IgG. In some embodiments, the polypeptide comprises Leu at the amino acid position that corresponds to amino acid position 377 of the wild type feline IgG. In some embodiments, the polypeptide comprises Tyr at the amino acid position that corresponds to amino acid position 377 of the wild type feline IgG. In some embodiments, the amino acid substitution at the position that corresponds to amino acid position 377 of a wild type feline IgG is a conservative amino acid substitution of Leu or Tyr.

In some embodiments, the polypeptide comprises Asp or Glu at the amino acid position that corresponds to amino acid position 392 of the wild type feline IgG. In some embodiments, the polypeptide comprises Asp at the amino acid position that corresponds to amino acid position 392 of the wild type feline IgG. In some embodiments, the polypeptide comprises Glu at the amino acid position that corresponds to amino acid position 392 of the wild type feline IgG. In some embodiments, the amino acid substitution at the position that corresponds to amino acid position 392 of a wild type feline IgG is a conservative amino acid substitution of Asp or Glu.

In some embodiments, the polypeptide comprises:
(i) Leu at the amino acid position that corresponds to amino acid position 428 of the wild type feline IgG and Asp at the amino acid position that corresponds to amino acid position 286 of the wild type feline IgG;
(ii) Leu at the amino acid position that corresponds to amino acid position 428 of the wild type feline IgG and Leu at the amino acid position that corresponds to amino acid position 301 of the wild type feline IgG;
(iii) Leu at the amino acid position that corresponds to amino acid position 428 of the wild type feline IgG and Tyr at the amino acid position that corresponds to amino acid position 301 of the wild type feline IgG;
(iv) Leu at the amino acid position that corresponds to amino acid position 428 of the wild type feline IgG and Val at the amino acid position that corresponds to amino acid position 301 of the wild type feline IgG;
(v) Leu at the amino acid position that corresponds to amino acid position 428 of the wild type feline IgG and Asp at the amino acid position that corresponds to amino acid position 309 of the wild type feline IgG;
(vi) Leu at the amino acid position that corresponds to amino acid position 428 of the wild type feline IgG and Leu at the amino acid position that corresponds to amino acid position 377 of the wild type feline IgG;
(vii) Leu at the amino acid position that corresponds to amino acid position 252 of the wild type feline IgG and Tyr at the amino acid position that corresponds to amino acid position 377 of the wild type feline IgG;
(viii) Leu at the amino acid position that corresponds to amino acid position 252 of the wild type feline IgG and Asp at the amino acid position that corresponds to amino acid position 392 of the wild type feline IgG; or
(ix) Leu at the amino acid position that corresponds to amino acid position 252 of the wild type feline IgG and Glu at the amino acid position that corresponds to amino acid position 392 of the wild type feline IgG.

In some embodiments of any one of the preceding aspects, the wild type feline IgG is a feline IgG1a comprising an Fc domain having an amino acid sequence of SEQ ID NO: 1, a feline IgG1b comprising an Fc domain having an amino acid sequence of SEQ ID NO: 2, or a feline IgG2 comprising an Fc domain having an amino acid sequence of SEQ ID NO: 3. In some embodiments, the wild type feline IgG is a feline IgG1a comprising an Fc domain having the amino acid sequence of SEQ ID NO: 1. In some embodiments, the wild type feline IgG is a feline IgG1b comprising an Fc domain having the amino acid sequence of SEQ ID NO: 2. In some embodiments, the wild type feline IgG is a feline IgG2 comprising an Fc domain having the amino acid sequence of SEQ ID NO: 3.

In some embodiments, the polypeptide comprises at least one amino acid substitution at a position corresponding to one or more of amino acid positions 252, 286, 301, 309, 311, 377, 392, and 428 of the wild type feline IgG, wherein the amino acid positions are based on EU numbering, and wherein the polypeptide has increased binding to feline FcRn compared to an Fc domain of the wild type feline IgG. The at least one amino acid substitution encompassed by the present disclosure can include one or more (e.g., 1, 2, 3, 4, 5, 6, 7, or 8) of those disclosed in Table 3.

TABLE 3

| Position | | Feline | Feline | Feline | |
|---|---|---|---|---|---|
| (EU Numbering) | hIgG1 | IgG1a | IgG1b | IgG2 | Substitution |
| CH2 Region | | | | | |
| 252 | M | S | S | S | Y, M |
| 286 | N | T | T | T | D, E |
| 301 | R | R | R | R | L, V, Y |
| 309 | L | L | L | L | V, E |
| 311 | Q | Q | Q | Q | V |
| CH3 Region | | | | | |
| 377 | I | I | I | I | Y, L |
| 392 | K | R | R | Q | D, E |
| 428 | M | S | S | S | M, L |

In some embodiments of any one of the preceding aspects, the polypeptide binds to the feline FcRn at a higher level at an acidic pH (e.g., pH 5.5, pH 6.0 or pH 6.5) than at a neutral pH (e.g., pH 7.0, 7.1, 7.2, 7.3, 7.4, or 7.5).

In some embodiments, the polypeptide binds to the feline FcRn at a higher level at a pH of 5.5 to 6.0 than at pH 7.4. In some embodiments, the polypeptide binds to the feline FcRn at a higher level at a pH of 5.5 than at pH 7.4. In some embodiments, the polypeptide binds to the feline FcRn at a higher level at a pH 6.0 than at pH 7.4.

Any of the polypeptides disclosed herein may comprise one or more additional amino acid substitutions, including any amino acid substitutions as disclosed in U.S. Patent Application Publication No. 2022/0259282, U.S. patent application Ser. No. 18/046,082, and U.S. Pat. No. 11,498,953, each of which is incorporated herein by reference in its entirety.

The present disclosure provides a polypeptide comprising a feline IgG Fc region variant, or a feline FcRn-binding region thereof, wherein the polypeptide comprises an amino acid substitution at at least one position selected from the group consisting of:
(i) a position that corresponds to amino acid position 252 of a wild type feline IgG, wherein the amino acid substitution is S252W;
(ii) a position that corresponds to amino acid position 254 of a wild type feline IgG, wherein the amino acid substitution is selected from the group consisting of S254R and S254K;
(iii) a position that corresponds to amino acid position 309 of a wild type feline IgG, wherein the amino acid substitution is L309V or L309Y;
(iv) a position that corresponds to amino acid position 311 of a wild type feline IgG, wherein the amino acid substitution is selected from the group consisting of Q311R, Q311V, Q311L and Q311K;
(v) a position that corresponds to amino acid position 428 of a wild type feline IgG, wherein the amino acid substitution is selected from the group consisting of S428M, S428Y, S428H and S428R; and
(vi) one or more positions that correspond to amino acid positions selected from the group consisting of 262, 286, 289, 290, 293, 301, 312, 326, 334, 347, 355, 377, 380, 383, 389c, 392, 426 and 437 of a wild type feline IgG;
wherein the amino acid positions are based on EU numbering, and wherein the polypeptide has increased binding affinity to feline FcRn when compared to an Fc domain of the wild type feline IgG.

In some embodiments, the polypeptide has increased binding affinity to feline FcRn at a pH of about 5.0 to about 6.5 (e.g., about 5.5 or about 6.0) when compared to an Fc domain of the wild type feline IgG.

In some embodiments, the polypeptide comprises the amino acid substitution at a position that corresponds to amino acid position 252 of a wild type feline IgG. In some embodiments, the amino acid substitution at position 252 of the wild type feline IgG is S252W.

In some embodiments, the polypeptide comprises the amino acid substitution at a position that corresponds to amino acid position 254 of a wild type feline IgG. In some embodiments, the amino acid substitution at position 254 of the wild type feline IgG is S254R. In some embodiments, the amino acid substitution at position 254 of the wild type feline IgG is S254K.

In some embodiments, the polypeptide comprises amino acid substitution L309V or L309Y.

In some embodiments, the polypeptide comprises the amino acid substitution at a position that corresponds to amino acid position 311 of a wild type feline IgG. In some embodiments, the amino acid substitution at position 311 of the wild type feline IgG is Q311R. In some embodiments, the amino acid substitution at position 311 of the wild type feline IgG is Q311V. In some embodiments, the amino acid substitution at position 311 of the wild type feline IgG is Q311K. In some embodiments, the amino acid substitution at position 311 of the wild type feline IgG is Q311L.

In some embodiments, the polypeptide comprises the amino acid substitution at a position that corresponds to amino acid position 428 of a wild type feline IgG. In some embodiments, the amino acid substitution at position 428 of the wild type feline IgG is S428M.

In some embodiments, the polypeptide comprises at least the amino acid substitution S428Y. In some embodiments, the amino acid substitution at position 428 of the wild type feline IgG is S428Y. In some embodiments, the amino acid substitution at position 428 of the wild type feline IgG is S428R. In some embodiments, the amino acid substitution at position 428 of the wild type feline IgG is S428H.

In another embodiment, the polypeptide comprises an amino acid substitution at one or more positions that correspond to amino acid positions selected from the group consisting of 262, 286, 289, 290, 293, 301, 312, 326, 334, 347, 355, 377, 380, 383, 389c, 392, 426 and 437 of a wild type feline IgG.

In some embodiments, the amino acid substitution is selected from the group consisting of L262Q, L262E, T286E, T286D, T289K, S290V, S290Y, E293D, E293H, E293K, R301L, D312T, K326D, R334D, Q347L, Q355L, I377V, I377Y, E380D, E380V, E380T, I383L, N389c-R, R392E, S426L, S426H and T437L, and conservative amino acid substitutions of any of foregoing. In some embodiments, the amino acid substitution is selected from the group consisting of L262Q, L262E, T286E, T286D, T289K, S290V, S290Y, E293D, E293H, E293K, R301L, D312T, K326D, R334D, Q347L, Q355L, I377V, 1377Y, E380D, E380V, E380T, I383L, N389c-R, R392E, S426L, S426H and T437L.

In another aspect, the disclosure provides a polypeptide comprising a feline IgG Fc region variant, or a feline FcRn-binding region thereof, wherein the polypeptide comprises two or more amino acid substitutions, wherein the two or more amino acid substitutions are selected from the group consisting of:
(i) an amino acid substitution at a position that corresponds to amino acid position 252 of a wild type feline IgG, wherein the amino acid substitution is selected from the group consisting of S252W, S252Y, S252F and S252R;
(ii) an amino acid substitution at a position that corresponds to amino acid position 254 of a wild type feline IgG, wherein the amino acid substitution is selected from the group consisting of S254R and S254K;
(iii) an amino acid substitution at a position that corresponds to amino acid position 309 of a wild type feline IgG, wherein the amino acid substitution is selected from the group consisting of L309V, L309Y and L309E;
(iv) an amino acid substitution at a position that corresponds to amino acid position 311 of a wild type feline IgG, wherein the amino acid substitution is selected from the group consisting of Q311R, Q311V, Q311L and Q311K;
(v) an amino acid substitution at a position that corresponds to amino acid position 428 of a wild type feline IgG, wherein the amino acid substitution is selected from the group consisting of S428L, S428M, S428Y, S428H and S428R;
(vi) an amino acid substitution at one or more positions that correspond to amino acid positions selected from the group consisting of 262, 286, 289, 290, 293, 301, 312, 326, 334, 347, 355, 377, 380, 383, 389c, 392, 426 and 437 of a wild type feline IgG; and
(vii) an amino acid substitution at a position that corresponds to amino acid position 434 of a wild type feline IgG, wherein the amino acid substitution is selected from the group consisting of S434F, S434W, S434H, S434R, and S434Y;
wherein the amino acid positions are based on EU numbering, wherein the two or more amino acid substitutions are at different positions, and wherein the polypeptide has increased binding affinity to feline FcRn when compared to (a) an Fc domain of the wild type feline IgG, and (b) a polypeptide comprising only one of the two or more amino acid substitutions.

In some embodiments, the two or more amino acid substitutions comprise an amino acid substitution at a position that corresponds to amino acid position 286 of a wild type feline IgG, wherein the amino acid substitution is selected from the group consisting of T286E and T286D.

In some embodiments, the two or more amino acid substitutions comprise an amino acid substitution at a position that corresponds to amino acid position 289 of a wild type feline IgG, wherein the amino acid substitution is selected from the group consisting of T289K and T289H.

In some embodiments, the two or more amino acid substitutions comprise an amino acid substitution at a position that corresponds to amino acid position 301 of a wild type feline IgG, wherein the amino acid substitution is R301L.

In some embodiments, the two or more amino acid substitutions comprise an amino acid substitution at a position that corresponds to amino acid position 334 of a wild type feline IgG, wherein the amino acid substitution is R334D.

In some embodiments, the two or more amino acid substitutions comprise an amino acid substitution at a position that corresponds to amino acid position 426 of a wild type feline IgG, wherein the amino acid substitution is selected from the group consisting of S426L and S426H.

In some embodiments, the two or more amino acid substitutions comprise an amino acid substitution at a position that corresponds to amino acid position 437 of a wild type feline IgG, wherein the amino acid substitution is T437L.

In some embodiments, the two or more amino acid substitutions are selected from the group consisting of:
(i) S252Y in combination with Q311R and/or Q311L;
(ii) S434Y in combination with one or more of S254R, S254K, L262E, T286D, T286E, T289K, E293D, E293K, L309V, L309E, K326D and Q347L;
(iii) S434F and E380D;
(iv) S428L in combination with one or more of S252R, T286E, Q311V, Q311K, D312T, I377V, I383L, N389cR;
(v) S428L, E380D and S434R;
(vi) S428L, E380T and S434R;
(vii) S252R in combination with L262Q;
(viii) T260E, L309E and Q355L;
(ix) S290V and R344D; and
(x) R301L, E380V and T437L.

In some embodiments, the two or more amino acid substitutions are T286E, Q311V, and S428Y.

In some embodiments, the polypeptide comprises an amino acid sequence that is at least 80% (e.g., at least 85%, 90%, 92%, 94%, 95%, 96%, 97%, 98%, or 99%) identical to an amino acid sequence selected from the group consisting of SEQ ID NOs: 1 to 3.

In some instances, this disclosure provides a feline IgG CH2 region variant comprising an amino acid sequence that is at least 75%, at least 80%, at least 85%, at least 90%, at least 95%, at least 96%, at least 97%, at least 98% or at least 99% identical to the amino acid sequence set forth in any one of SEQ ID NOs: 4 to 6. Also provided are feline IgG CH2 region variants comprising an amino acid sequence that varies from any one of SEQ ID NOs: 4 to 6 by 1 to 15 (e.g., 1, 2, 3, 4, 5, 6, 7, 8, 9, 10, 11, 12, 13, 14, or 15) amino acids.

In other instances, this disclosure features a feline IgG CH3 region variant comprising an amino acid sequence that is at least 75%, at least 80%, at least 85%, at least 90%, at least 95%, at least 96%, at least 97% or at least 98% or at least 99%, identical to the amino acid sequence set forth in any one of SEQ ID NOs: 7 to 9. Also featured are feline IgG CH3 region variants comprising an amino acid sequence that varies from any one of SEQ ID NOs: 7 to 9 by 1 to 15 (e.g., 1, 2, 3, 4, 5, 6, 7, 8, 9, 10, 11, 12, 13, 14, or 15) amino acids.

In certain instances, this disclosure features a feline IgG Fc region variant comprising an amino acid sequence that is at least 75%, at least 80%, at least 85%, at least 90%, at least 95%, at least 96%, at least 97%, at least 98% or at least 99% identical to the amino acid sequence set forth in any one of SEQ ID NOs: 1 to 3. Also disclosed are feline IgG Fc region variants comprising an amino acid sequence that varies from any one of SEQ ID NOs: 1 to 3 by 1 to 20 (e.g., 1, 2, 3, 4, 5, 6, 7, 8, 9, 10, 11, 12, 13, 14, 15, 16, 17, 18, 19, or 20) amino acids.

In some embodiments, provided are a polypeptide or polypeptides comprising a feline IgG Fc CH2 region variant, the CH2 region variant comprising an amino acid sequence that is at least 75%, at least 80%, at least 85%, at least 90%, at least 95%, at least 96%, at least 97%, at least 98% or at least 99%, identical to the amino acid sequence set forth in any one of SEQ ID NOs: 4 to 6.

In some embodiments, featured are a polypeptide or polypeptides comprising a feline IgG Fc CH3 region variant, the CH3 region variant comprising an amino acid sequence that is at least 75%, at least 80%, at least 85%, at least 90%, at least 95%, at least 96%, at least 97%, at least 98% or at least 99% identical to the amino acid sequence set forth in any one of SEQ ID NOs: 7 to 9.

In some embodiments, featured are a polypeptide or polypeptides comprising a feline IgG Fc region variant, the Fc region variant comprising an amino acid sequence that is at least 75%, at least 80%, at least 85%, at least 90%, at least 95%, at least 96%, at least 97%, at least 98% or at least 99% identical to the amino acid sequence set forth in any one of SEQ ID NOs: 1 to 3.

As noted elsewhere, the polypeptide, in some embodiments, further comprises at least one additional amino acid substitution in a region corresponding to amino acid positions 250-256, amino acid positions 285-288; amino acid positions 307-315; amino acid positions 376-380, amino acid positions 383 to 392; or amino acid positions 428-437 of the wild type feline IgG, wherein the amino acid positions are based on EU numbering, and wherein the polypeptide has increased binding to feline FcRn compared to an Fc domain of the wild type feline IgG.

In some embodiments, the polypeptide comprises at least one (e.g., 1, 2, 3, 4, 5, 6, 7, 8, 9, 10, 11, 12, 13, 14, 15, 16, 17, 18, 19, or 20) additional amino acid substitution selected from those disclosed in Table 4, below.

TABLE 4
List of amino acid substitutions (Groups 1 and 2) that increase
binding of the feline IgG1a Fc variant to feline FcRN

| | | Feline IgG1a Fc variant | Alternative feline IgG1a Fc |
|---|---|---|---|
| Position by EU | Wild type | amino acid substitutions | variant amino acid |
| numbering | feline IgG1a Fc | (Group 1) | substitutions (Group 2) |
| 252 | S | W | FYMRV |
| 254 | S | RK | WYHLFVM |
| 262 | L | QE | |
| 286 | T | ED | |
| 289 | T | K | |
| 290 | S | VY | |
| 293 | E | DKH | |
| 301 | R | L | |
| 309 | L | VY | E |
| 311 | Q | RVKL | AYF |
| 312 | D | T | |
| 326 | K | D | |
| 334 | R | D | |
| 347 | Q | L | |
| 355 | Q | L | |
| 377 | I | VY | |
| 380 | E | DVT | |
| 383 | I | L | |
| 389c | N | R | |
| 392 | R | E | |
| 426 | S | LH | |
| 428 | S | RMYH | WL |
| 434 | S | RYFWH | A |
| 437 | T | L | |

The amino acid substitutions may be made on one or both chains of a CH2 domain, a CH3 domain, or an Fc domain. In some instances, the substitutions on both chains of a CH2 domain, a CH3 domain, or an Fc domain are identical. In some instances, the substitutions on both chains of a CH2 domain, a CH3 domain, or an Fc domain are not identical. In some instances, the Fc region includes one or more additional substitutions that increase or decrease effector function and/or improve product heterogeneity.

Other Substitutions that can be Combined with the Half-Life Enhancing Substitutions

The development of a therapeutic polypeptide/protein (e.g., a monoclonal antibody) is a complex process that entails coordination of a complex set of activities to generate the desired polypeptide/protein. These include optimization of the specificity, affinity, functional activity, expression level in engineered cell lines, long-term stability, elimination or enhancement of effector functions and development of commercially viable manufacturing and purification methods. This disclosure encompasses substitutions at one or more additional amino acid positions of the Fc region variant that facilitates any one or more of the above goals.

In some embodiments, the Fc region variant comprises amino acid substitutions at one or more additional amino acid positions that increase or decrease effector function and/or improve product heterogeneity.

In some embodiments, the substitutions are introduced to reduce effector function of the feline Fc region. Such substitutions will be familiar to persons skilled in the art and may be at one or more (e.g., 1, 2, 3, 4, 5, 6, or 7) positions of the feline IgG. Illustrative examples include WO 2019/035010 A1.

In some embodiments, substitutions are introduced to a wild type feline IgG Fc region to enhance binding to Protein A so as to facilitate purification by protein A chromatography. Such substitutions may be at one or more (e.g., 1, 2, 3, 4, 5, 6, or 7) positions of the feline IgG. Illustrative examples include WO 2019/035010 A1.

In some embodiments, additional amino acid substitutions can be made to alter binding affinity to FcRn as compared to a parent polypeptide or a wild type polypeptide (e.g., to increase or reduce binding affinity with FcRn). In some embodiments, the substitutions are made to alter binding affinity to FcRn as compared to a parent polypeptide or a wildtype polypeptide (e.g., to increase or reduce binding affinity with FcRn). In some variations, the modification can be one, two, three, or four modifications that are selected from the group consisting of: 308F, 428L, 434M and 434S, where the numbering is according to the EU numbering. In some embodiments, the Fc variant includes one or more modifications selected from the group consisting of: 252Y/428L, 428L/434H, 428L/434F, 428L/434Y, 428L/434A, 428L/434M, and 428L/434S, where the numbering is according to the EU numbering. In some embodiments, the Fc variant includes one or more modification selected from the group consisting of: 428L/434S, 308F/428L/434S, where the numbering is according to the EU numbering. In some embodiments, the Fc variant includes one or more modifications selected from the group consisting of: 2591/434S, 308F/434S, 308F/428L/434S, 2591/308F/434S, 307Q/308F/434S, 2501/308F/434S, and 308F/319L/434S, where the numbering is according to the EU numbering. A detailed description of these modifications is described in e.g., U.S. Pat. No. 8,883,973B2, which is incorporated herein by reference in its entirety.

In some embodiments, the polypeptide comprises a hinge region of a feline antibody. In some embodiments, modifications can be made to the hinge region of the feline antibody to increase half-life. In some embodiments, the modification is 228P according to EU numbering.

In some embodiments, the binding with FcRn is pH-dependent. H310 and H435 (EU numbering) can be critical for pH-dependent binding. Thus, in some embodiments, the amino acids at position 310 (EU numbering) is histidine. In some embodiments, the amino acids at position 435 (EU numbering) is histidine. In some embodiments, the amino acids at both positions are histidine.

In some embodiments, the Fc region has LALA mutations (L234A and L235A mutations in EU numbering), or LALA-PG mutations (L234A, L235A, P329G mutations in EU numbering). In some embodiments, the LALA mutation is P234A, M234A, or S234A. In some embodiments, the amino acid residue at position 234 (EU numbering) is Ala. In some embodiments, the amino acid residue at position 234 (EU numbering) is Ala. In some embodiments, the amino acid residues at positions 234 and 235 (EU numbering) are Ala.

### Polypeptides Comprising the Feline IgG Fc Variants

The disclosure encompasses any polypeptide that may benefit from having an increased half-life in a cat. To increase half-life, these polypeptides are designed to include an Fc region variant (e.g., a CH2 region, a CH3 region, or a CH2+CH3 region) disclosed above.

In some embodiments, the polypeptides of this disclosure include an antibody hinge region. The hinge region may be placed between the antigen or ligand-binding domain of the polypeptide and the Fc region variant. In some instances, the hinge region is attached to the C-terminus of a cytokine, a growth factor, an enzyme, or a peptide and the hinge region is attached to the N-terminus of the Fc region variant. Exemplary hinge region sequences are provided below.

IgG1a:
(SEQ ID NO: 10)
KTDHPPGPKPCDCPKCP;
IgG1b:
(SEQ ID NO: 11)
KTDHPPGPKPCDCPKCP;
and
IgG2:
(SEQ ID NO: 12)
KTASTIESKTGEGPKCP;

The hinge region, if used, in a recombinant protein of this disclosure may include zero to six (i.e., 0, 1, 2, 3, 4, 5, or 6) amino acid substitutions relative to an amino acid sequence set forth in any one of SEQ ID NOs: 10-12. In some instances, the hinge region used in a recombinant protein of this disclosure is at least 80%, at least 85%, at least 90%, at least 95%, at least 96%, at least 97%, at least 98%, at least 99%, or 100% identical to an amino acid sequence set forth in any one of SEQ ID NOs: 10-12.

In some embodiments, a linker sequence may be used instead of an antibody hinge sequence to connect the polypeptide (e.g., antibodies, ligand-binding domains of receptors, enzymes, ligands, peptides) to the feline Fc region variants disclosed herein. In certain embodiments, the linker is made up of from 1 to 20 amino acids linked by peptide bonds, wherein the amino acids are selected from the 20 naturally occurring amino acids. Some of these amino acids may be glycosylated, as is well understood by those in the art. In other embodiments, the 1 to 20 amino acids are selected from glycine, alanine, proline, asparagine, glutamine, and lysine. In other embodiments, a linker is made up of a majority of amino acids that are sterically unhindered, such as glycine and alanine. Examples of peptide linkers include: Gly, Ser; Gly Ser; Gly Gly Ser; Ser Gly Gly; Gly Gly Gly Ser (SEQ ID NO: 13); Ser Gly Gly Gly (SEQ ID NO: 14); Gly Gly Gly Gly Ser (SEQ ID NO: 15); Ser Gly Gly Gly Gly (SEQ ID NO: 16); Gly Gly Gly Gly Gly Ser (SEQ ID NO: 17); Ser Gly Gly Gly Gly Gly (SEQ ID NO: 18); Gly Gly Gly Gly Gly Gly Ser (SEQ ID NO: 19); Ser Gly Gly Gly Gly Gly Gly (SEQ ID NO: 20); (Gly Gly Gly Gly Ser)ₙ (SEQ ID NO: 15), wherein n is an integer of one or more (e.g., 1, 2, 3, 4, 5); and (Ser Gly Gly Gly Gly)ₙ (SEQ ID NO: 16), wherein n is an integer of one or more (e.g., 1, 2, 3, 4, 5).

Non-peptide linkers may also be used to link the polypeptide or polypeptides of interest to an Fc region variant disclosed herein. For example, alkyl linkers such as —NH(CH₂)ₙC(O)—, wherein n=2-20 can be used. These alkyl linkers may further be substituted by any non-sterically hindering group such as lower alkyl (e.g., C₁-C₆) lower acyl, halogen (e.g., Cl, Br), CN, NH₂, phenyl, etc.

The polypeptide or polypeptides of this disclosure may comprise a binding domain. The binding domain can specifically bind to a protein, subunit, domain, motif, and/or epitope of a selected target described herein. In some embodiments, the binding domain comprises an antibody, an antibody fragment, or a ligand-binding portion of a receptor. In some embodiments, the antibody or the antibody fragment comprises six complementarity determining regions (CDRs) of an immunoglobulin molecule. In other embodiments, the antibody fragment is selected from the group consisting of Fab, single chain variable fragment (scFv), Fv, Fab′, Fab′-SH, F(ab′)2, nanobody, and diabody. In other embodiments, the ligand-binding portion of a receptor comprises a ligand binding domain of a feline receptor protein or an extracellular domain of a feline receptor protein. In some embodiments, the polypeptide or polypeptides (e.g., fusion polypeptide) can comprise a protein, wherein the protein is a therapeutic protein described herein. In some embodiments, the target (e.g., for the target of the binding domain) or the therapeutic protein (e.g., for the fusion polypeptide) is selected from the group consisting of: 17-IA, 4-1BB, 4Dc, 6-keto-PGF1a, 8-iso-PGF2a, 8-oxo-dG, A1 Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RIIB, ADAM, ADAM10, ADAM12, ADAM15, ADAM17/TACE, ADAMS, ADAM9, ADAMTS, ADAMTS4, ADAMTS5, Addressins, aFGF, ALCAM, ALK, ALK-1, ALK-7, alpha-1-antitrypsin, alpha-V/beta-1 antagonist, ANG, Ang, APAF-1, APE, APJ, APP, APRIL, AR, IgE, Angiotensin type 1 (AT1) receptor, Angiotensin type 2 (AT2) receptor, ARC, ART, Artemin, anti-Id, ASPARTIC, Atrial natriuretic factor, av/b3 integrin, Axl, b2M, B7-1, B7-2, B7-H, B-lymphocyte Stimulator (BlyS), BACE, BACE-1, Bad, BAFF, BAFF-R, Bag-1, BAK, Bax, BCA-1, BCAM, Bcl, BCMA, BDNF, b-ECGF, bFGF, BID, Bik, BIM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-DNA, BTC, complement factor 3 (C3), C3a, C4, C5, C5a, C10, CA125, CAD-8, Calcitonin, CAMP, carcinoembryonic antigen (CEA), carcinoma-associated antigen, Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/Z/P, CBL, CC1, CCK2, CCL, CCL1, CCL11, CCL12, CCL13, CCL14, CCL15, CCL16, CCL17, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD3, CD3E, CD4, CD5, CD6, CD7, CD8, CD10, CD11a, CD11b, CD11c, CD13, CD14, CD15, CD16, CD18, CD19, CD20, CD21, CD22, CD23, CD25, CD27L, CD28, CD29, CD30, CD30L, CD32, CD33 (p67 proteins), CD34, CD38, CD40, CD40L, CD44, CD45, CD46, CD47, CD49a, CD52, CD54, CD55, CD56, CD61, CD64, CD66e, CD74, CD80 (B7-1), CD89, CD95, CD123, CD137, CD138, CD140a, CD146, CD147, CD148, CD152, CD164, CEACAM5, CFTR, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigen, DAN, DCC, DcR3, DC-SIGN, Decay accelerating factor, des(1-3)-IGF-I (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, ENA, endothelin receptor, Enkephalinase, eNOS, Eot, eotaxin1, EpCAM, Ephrin B2/EphB4, EPO, ERCC, E-selectin, ET-1, Factor 11a, Factor VII, Factor VIIIc, Factor IX, fibroblast activation protein (FAP), Fas, FcR1, FEN-1, Ferritin, FGF, FGF-19, FGF-2, FGF3, FGF-8, FGFR, FGFR-3, Fibrin, FL, FLIP, Flt-3, Flt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GDNF, GFAP, GFRa-1, GFR-alpha1, GFR-alpha2, GFR-alpha3, GITR, GLP1, GLP2, Glucagon, Glut 4, glycoprotein IIb/IIIa (GP IIb/IIIa), GM-CSF, gp130, gp72, GRO, GnRH, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gp120, heparanase, Her2, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MAA), HIV gp120, HIV IIIB gp120 V3 loop, HLA, HLA-DR, HM1.24, HMFG PEM, HRG, Hrk, cardiac myosin, cytomegalovirus (CMV), growth hormone (GH), HVEM, 1-309, IAP, ICAM, ICAM-1, ICAM-3, ICE, ICOS, IFNg, Ig, IgA receptor, IgE, IGF, IGF binding proteins, IGF-1R, IGFBP, IGF-I, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-17, IL-18, IL-18R, IL-21, IL-22, IL-23, IL-25, IL-31, IL-33, interleukin receptor (e.g., IL-1R, IL-2R, IL-4R, IL-5R, IL-6R, IL-8R, IL-9R, IL-10R, IL-12R, IL-13R, IL-15R, IL-17R, IL-18R, IL-21R, IL-22R, IL-23R, IL-25R, IL-31R, IL-33R), interferon (INF)-alpha, INF-beta, INF-gamma, Inhibin, iNOS, Insulin A-chain, Insulin B-chain, Insulin-like growth factor 1, integrin alpha2, integrin alpha3, integrin alpha4, integrin alpha4/beta1, integrin alpha4/beta7, integrin alpha5 (alphaV), integrin alpha5/beta1, integrin alpha5/beta3, integrin alpha6, integrin beta1, integrin beta2, interferon gamma, IP-10, I-TAC, JE, Kallikrein 2, Kallikrein 5, Kallikrein 6, Kallikrein 11, Kallikrein 12, Kallikrein 14, Kallikrein 15, Kallikrein L1, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bp1, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, METALLOPROTEASES, MGDF receptor, MGMT, MHC(HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Muc1), MUC18, Muellerian-inhibitin substance, Mug, MuSK, NAIP, NAP, NAV 1.7, NCAD, N-Cadherin, NCA 90, NCAM, NCAM, Neprilysin, Neurotrophin-3, -4, or -6, Neurturin, Neuronal growth factor (NGF), NGFR, NGF-beta, nNOS, NO, NOS, Npn, NRG-3, NT, NTN, OB, OGG1, oncostatin M receptor (OSMR), OPG, OPN, OSM, OX40L, OX40R, p150, p95, PADPr, Parathyroid hormone, PARC, PARP, PBR, PBSF, PCAD, P-Cadherin, PCNA, PD1, PDL1, PDGF, PDGF, PDK-1, PECAM, PEM, PF4, PGE, PGF, PGI2, PGJ2, PIN, PLA2, placental alkaline phosphatase (PLAP), P1GF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, RANTES, Relaxin A-chain, Relaxin B-chain, renin, respiratory syncytial virus (RSV) F, RSV Fgp, Ret, Rheumatoid factors, RLIP76, RPA2, RSK, S100, SCF/KL, SDF-1, SERINE, Serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T-cell receptors (e.g., T-cell receptor alpha/beta), TdT, TECK, TEM1, TEM5, TEM7, TEM8, TERT, testicular PLAP-like alkaline phosphatase, TfR, TGF, TGF-alpha, TGF-beta, TGF-beta Pan Specific, TGF-beta R1 (ALK-5), TGF-beta R11, TGF-beta Rllb, TGF-beta RIII, TGF-beta1, TGF-beta2, TGF-beta3, TGF-beta4, TGF-beta5, Thrombin, Thymus Ck-1, Thyroid stimulating hormone, Tie, TIMP, TIQ, Tissue Factor, TMEFF2, Tmpo, TMPRSS2, TNF, TNF-alpha, TNF-alpha beta, TNF-beta2, TNFc, TNF-RI, TNF-RII, TNFRSF10A (TRAIL R1Apo-2, DR4), TNFRSF10B (TRAIL R2DR5, KILLER, TRICK-2A, TRICK-B), TNFRSF10C (TRAIL R3DcR1, LIT, TRID), TNFRSF10D (TRAIL R4 DcR2, TRUNDD), TNFRSF11A (RANK ODF R, TRANCE R), TNFRSF11B (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF18 (GITR AITR), TNFRSF19 (TROY TAJ, TRADE), TNFRSF19L (RELT), TNFRSF1A (TNF R1CD120a, p55-60), TNFRSF1B (TNF RII CD120b, p75-80), TNFRSF26 (TNFRH3), TNFRSF3 (LTbR TNF RIII, TNFC R), TNFRSF4 (OX40 ACT35, TXGP1 R), TNFRSF5 (CD40 p50), TNFRSF6 (Fas Apo-1, APT1, CD95), TNFRSF6B (DcR3M68, TR6), TNFRSF7 (CD27), TNFRSF8 (CD30), TNFRSF9 (4-1BB CD137, ILA), TNFRSF21 (DR6), TNFRSF22 (DCTRAIL R2 TNFRH2), TNFRST23 (DCTRAIL R1TNFRH1), TNFRSF25 (DR3Apo-3, LARD, TR-3, TRAMP, WSL-1), TNFSF10 (TRAIL Apo-2 Ligand, TL2), TNFSF11 (TRANCE/RANK Ligand ODF, OPG Ligand), TNFSF12 (TWEAK Apo-3 Ligand, DR3Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TL1A/VEGI), TNFSF18 (GITR Ligand AITR Ligand, TL6), TNFSF1A (TNF-α Conectin, DIF, TNFSF2), TNFSF1B (TNF-b LTa, TNFSF1), TNFSF3 (LTb TNFC, p33), TNFSF4 (OX40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSF8 (CD30 Ligand CD153), TNFSF9 (4-1BB Ligand CD137 Ligand), TP-1, t-PA, Tpo, TRAIL, TRAIL R, TRAIL-R1, TRAIL-R2, TRANCE, transferring receptor, TRF, Trk (e.g., TrkA), TROP-2, TSG, TSLP, tumor-associated antigen CA 125, tumor-associated antigen expressing Lewis Y related carbohydrate, TWEAK, TXB2, Ung, UPAR, uPAR-1, Urokinase, VCAM, VCAM-1, VECAD, VE-Cadherin, VE-cadherin-2, VEFGR-1 (fit-1), VEGF, VEGFR, VEGFR-3 (flt-4), VEGI, VIM, Viral antigens, VLA, VLA-1, VLA-4, VNR integrin, von Willebrands factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, XIAP, XPD, and receptors for hormones and growth factor.

In some embodiments, the antibody or the antibody fragment comprises one or more complementarity determining regions (CDRs) with amino acid sequences selected from Table 5 below. For example, the antibody or the antibody fragment may include a CDR-H1, a CDR-H2, a CDR-H3, a CDR-L1, a CDR-L2, and a CDR-L3 selected from Table 5. For example, the antibody or the antibody fragment may include all six CDRs for an antibody that is listed as binding to a particular target in Table 5. In some embodiments, the antibody or antibody fragment may be any antibody or antibody fragment disclosed in U.S. Patent Application Publication Nos. US 2020/0062840, US 2022/0119513, US 2022/0106391, US 2022/0177594, or US 2022/0127351; U.S. Pat. No. 9,328,164; and International Patent Application Publication Nos. WO 2020/056393 or WO 2023/097275.

TABLE 5
Exemplary CDR sequences for feline antibodies

| | | | SEQ |
|---|---|---|---|
| Target | Publication Number | Sequences | ID NO: |
| IL-31 | U.S. 2020/0062840 | CDR-H1: GDSITSGYW | 21 |
| | | CDR-H2: YISYSGITDYNPSLKS | 22 |
| | | CDR-H3: ARYGNYGYAMDY | 23 |
| | | CDR-L1: RASESVDTYGNSFMH | 24 |
| | | CDR-L2: RASNLES | 25 |
| | | CDR-L3: QQSYEDPWT | 26 |
| IL-31 | U.S. 2020/0062840 | CDR-H1: GDSITSGYW | 21 |
| | | CDR-H2: YISYSGITYYNPSLKS | 27 |
| | | CDR-H3: ARYGNYGYAMDY | 23 |
| | | CDR-L1: RASESVDTYGNSFIH | 28 |
| | | CDR-L2: RASNLES | 25 |
| | | CDR-L3: QQSYEDPWT | 26 |
| IL-31 | U.S. 2020/0062840 | CDR-H1: GDSITSGYW | 21 |
| | | CDR-H2: YISYSGITDYNPSLKS | 22 |
| | | CDR-H3: ARYGNYGYAMDY | 23 |
| | | CDR-L1: RASESVDTYGNSFMH | 24 |
| | | CDR-L2: RASNLES | 25 |
| | | CDR-L3: QQSYEDPWT | 26 |
| IL-4R | WO 2020/056393 | CDR-H1: GYTFTSYVMH | 29 |
| | | CDR-H2: YINPKNDGTFYNGKFKG | 30 |
| | | CDR-H3: FNYGIAY | 31 |
| | | CDR-L1: RASQEISGYLS | 32 |
| | | CDR-L2: AASTLDS | 33 |
| | | CDR-L3: VQYASYPWT | 34 |
| IL-4R | WO 2020/056393 | CDR-H1: GYTFTSYVMH | 29 |
| | | CDR-H2: YINPNNDGTFYNGKFKG | 35 |
| | | CDR-H3: FYYGFAY | 36 |
| | | CDR-L1: RASQEISGYLS | 32 |
| | | CDR-L2: AASTLDS | 33 |
| | | CDR-L3: LQYASYPWT | 37 |
| TGF-β | U.S. 2022/0119513 | CDR-H1: SSWMN | 38 |
| | | CDR-H2: QIYPGDGDTNYNGKFKG | 39 |
| | | CDR-H3: ARHYDGSTDY | 40 |
| | | CDR-L1: RASENIYSNLA | 41 |
| | | CDR-L2: AATNLAD | 42 |
| | | CDR-L3: QHFWGTPYT | 43 |
| TGF-β | U.S. 2022/0119513 | CDR-H1: FSSYGMH | 44 |
| | | CDR-H2: VISYDGSIKYY | 45 |
| | | CDR-H3: TGEYSGYDTDPQYS | 46 |
| | | CDR-L1: RASQGIGDDLG | 47 |
| | | CDR-L2: GTSTLQS | 48 |
| | | CDR-L3: LQDSNYPLT | 49 |
| TGF-β | U.S. 2022/0119513 | CDR-H1: GYIFITY | 50 |
| | | CDR-H2: FPASGS | 51 |
| | | CDR-H3: GDGNYALDAMDY | 52 |
| | | CDR-L1: RASESVDSYGNSFMH | 53 |
| | | CDR-L2: LASNLES | 54 |
| | | CDR-L3: QQNNEDPLT | 55 |
| NGF | U.S. 2022/0106391 | CDR-H1: GFTLTQYG | 56 |
| | | CDR-H2: VIWATGATD | 57 |
| | | CDR-H3: DGWWYATSWYFDV | 58 |
| | | CDR-L1: KASQDINHYLN | 59 |
| | | CDR-L2: YTSRLHS | 60 |
| | | CDR-L3: QQGDHFPRT | 61 |
| NGF | WO 2023/097275 | CDR-H1: GLSLTSDS | 156 |
| | | CDR-H2: LWSNRGT | 157 |
| | | CDR-H3: ASIYYYEADYLHWYFDF | 158 |
| | | CDR-L1: EGIANN | 159 |
| | | CDR-L2: ATS | |
| | | CDR-L3: QQGFKWPLT | 161 |
| OSMR-β | U.S. 2022/0177594 | CDR-H1: DYGMH | 62 |
| | | CDR-H2: YISSGSRAVFFADTVKG | 63 |
| | | CDR-H3: DRYDGRGFAY | 64 |
| | | CDR-L1: RASQSISNNLH | 65 |
| | | CDR-L2: YASQSIS | 66 |
| | | CDR-L3: QQSNSWPLT | 67 |
| OSMR-β | U.S. 2022/0177594 | CDR-H1: SYAMS | 68 |
| | | CDR-H2: YISSGGDYIYYADTVKG | 69 |
| | | CDR-H3: DPITGTFAY | 70 |
| | | CDR-L1: RASQDINNYLN | 71 |
| | | CDR-L2: YTSTLHS | 72 |
| | | CDR-L3: QQGNTLPWT | 73 |
| OSMR-β | U.S. 2022/0177594 | CDR-H1: SYAMS | 68 |
| | | CDR-H2: YISSGGDYFYYADTVKG | 74 |
| | | CDR-H3: DPITGTFAY | 70 |
| | | CDR-L1: RASQDITNYLN | 75 |
| | | CDR-L2: YTSTLHS | 72 |
| | | CDR-L3: QQGHMLPWT | 76 |
| OSMR-β | U.S. 2022/0177594 | CDR-H1: DYYMA | 77 |
| | | CDR-H2: NINYDGSSTYYLDSLKS | 78 |
| | | CDR-H3: GLTWDFDV | 79 |
| | | CDR-L1: KASQDVDTAVA | 80 |
| | | CDR-L2: LASTRHT | 81 |
| | | CDR-L3: QQYSRFPLT | 82 |
| NGF | U.S. 9,328,164 | CDR-H1: NNNVN | 83 |
| | | CDR-H2: GVWAGGATDYNSALKGR | 84 |
| | | CDR-H3: DGGYSSSTLYAMDA | 85 |
| | | CDR-L1: RASEDIYNALA | 86 |
| | | CDR-L2: NTDTLHT | 87 |
| | | CDR-L3: HYFHYPRT | 88 |
| IL-31 | U.S. 2022/0127351 | CDR-H1: SYTIH | 89 |
| | | CDR-H2: NINPTSGYTENNQRFKD | 90 |
| | | CDR-H3: WGFKYDGEWSFDV | 91 |
| | | CDR-L1: RASQGISIWLS | 92 |
| | | CDR-L2: KASNLHI | 93 |
| | | CDR-L3: LQSQTYPLT | 94 |
| IL-31 | U.S. 2022/0127351 | CDR-H1: YYDIN | 95 |
| | | CDR-H2: WIFPGDGGTKYNETFKG | 96 |
| | | CDR-H3: ARGGTSVIRDAMDY | 97 |
| | | CDR-L1: RASESVDNYGISFMH | 98 |
| | | CDR-L2: RASNLES | 25 |
| | | CDR-L3: QQSNKDPLT | 99 |

In some embodiments, the binding domain specifically binds to one or more therapeutic targets or antigens in feline, such as, but are not limited to, ACE, ACE-2, Activin, Activin A, Activin AB, Activin B, Activin C, Activin RIA, Activin RIA ALK-2, Activin RIB ALK-4, Activin RIIA, Activin RIIB, ADAM, ADAM10, ADAM12, ADAM15, ADAM17/TACE, ADAMS, ADAM9, ADAMTS, ADAMTS4, ADAMTS5, ANG, Ang, Angiotensin type 1 (AT1) receptor, Angiotensin type 2 (AT2) receptor, Atrial natriuretic factor, av/b3 integrin, b-ECGF, CD19, CD20, CD30, CD34, CD40, CD40L, CD47, COX, CTLA-4, EGFR (ErbB-1), EPO, Follicle stimulating hormone, GDF-8 (Myostatin), GLP1, GLP2, GnRH, Growth hormone releasing factor, IgE, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-17, IL-18, IL-18R, IL-21, IL-22, IL-23, IL-25, IL-31, IL-33, interleukin receptor (e.g., IL-1R, IL-2R, IL-4R, IL-5R, IL-6R, IL-8R, IL-9R, IL-10R, IL-12R, IL-13R, IL-15R, IL-17R, IL-18R, IL-21R, IL-22R, IL-23R, IL-25R, IL-31R, IL-33R), LAP (TGF-1), Latent TGF-1, Latent TGF-1 bp1, LFA-1, Neuronal growth factor (NGF), NGFR, NGF-beta, OSMR, OX40L, OX40R, PD1, PDL1, TGF, TGF-alpha, TGF-beta, TGF-beta Pan Specific, TGF-beta R1 (ALK-5), TGF-beta R11, TGF-beta Rllb, TGF-beta RIII, TGF-beta1, TGF-beta2, TGF-beta3, TGF-beta4, TGF-beta5, TNF, TNF-alpha, TNF-alpha beta, TNF-beta2, TNFc, TNF-RI, TNF-RII, TNFRSF16 (NGFR p75NTR), TNFRSF9 (4-1BB CD137, ILA), VEFGR-1 (fit-1), VEGF, VEGFR, and VEGFR-3 (flt-4).

In some embodiments, the polypeptide or polypeptides can comprise a protein, wherein the protein is a therapeutic protein, e.g., EPO, CTLA4, LFA3, VEGFR1/VEGFR3, IL-1R, IL-4R, GLP-1 receptor agonist, or Thrombopoietin binding peptide. In some embodiments, the therapeutic protein is ACE, ACE-2, Activin, Activin A, Activin AB, Activin B, Activin C, Activin RIA, Activin RIA ALK-2, Activin RIB ALK-4, Activin RIIA, Activin RIIB, ADAM, ADAM10, ADAM12, ADAM15, ADAM17/TACE, ADAMS, ADAM9, ADAMTS, ADAMTS4, ADAMTS5, ANG, Ang, Angiotensin type 1 (AT1) receptor, Angiotensin type 2 (AT2) receptor, Atrial natriuretic factor, av/b3 integrin, b-ECGF, CD19, CD20, CD30, CD34, CD40, CD40L, CD47, COX, CTLA-4, EGFR (ErbB-1), EPO, Follicle stimulating hormone, GDF-8 (Myostatin), GLP1, GLP2, GnRH, Growth hormone releasing factor, IgE, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-17, IL-18, IL-18R, IL-21, IL-22, IL-23, IL-25, IL-31, IL-33, interleukin receptor (e.g., IL-1R, IL-2R, IL-4R, IL-5R, IL-6R, IL-8R, IL-9R, IL-10R, IL-12R, IL-13R, IL-15R, IL-17R, IL-18R, IL-21R, IL-22R, IL-23R, IL-25R, IL-31R, IL-33R), LAP (TGF-1), Latent TGF-1, Latent TGF-1 bp1, LFA-1, Neuronal growth factor (NGF), NGFR, NGF-beta, OSMR, OX40L, OX40R, PD1, PDL1, TGF, TGF-alpha, TGF-beta, TGF-beta Pan Specific, TGF-beta R1 (ALK-5), TGF-beta R11, TGF-beta RIIb, TGF-beta RIII, TGF-beta1, TGF-beta2, TGF-beta3, TGF-beta4, TGF-beta5, TNF, TNF-alpha, TNF-alpha beta, TNF-beta2, TNFc, TNF-RI, TNF-RII, TNFRSF16 (NGFR p75NTR), TNFRSF9 (4-1BB CD137, ILA), VEFGR-1 (fit-1), VEGF, VEGFR, or VEGFR-3 (flt-4).

For Example, the polypeptide or polypeptides of this disclosure may comprise a binding domain comprising six CDRs of an immunoglobulin molecule. In some embodiments, the binding domain specifically binds to NGF. In some embodiments, the binding domain is an antibody or antibody fragment. In some embodiments, the antibody or antibody fragment comprises a CDR-H1 comprising the amino acid sequence of SEQ ID NO: 156, a CDR-H2 comprising the amino acid sequence of SEQ ID NO: 157, a CDR-H3 comprising the amino acid sequence of SEQ ID NO: 158, a CDR-L1 comprising the amino acid sequence of SEQ ID NO: 159, a CDR-L2 comprising the amino acid sequence of ATS, and a CDR-L3 comprising the amino acid sequence of SEQ ID NO: 161.

In some embodiments, the polypeptide comprises a heavy chain and a light chain, wherein the heavy chain comprises an amino acid sequence having at least 80% (e.g., 85%, 90%, 95%, 97%, 98%, 99%, and 100%) sequence identity to any one of SEQ ID NOs: 101-150, and the light chain comprises an amino acid sequence having at least 80% (e.g., 85%, 90%, 95%, 97%, 98%, 99%, and 100%) sequence identity to SEQ ID NO: 100.

In some embodiments, the heavy chain comprises an amino acid sequence having at least 80% (e.g., 85%, 90%, 95%, 97%, 98%, 99%, and 100%) sequence identity to SEQ ID NO: 101, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Tyr at the amino acid position that corresponds to amino acid position 252 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 102, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Tyr at the amino acid position that corresponds to amino acid position 252 of the wild type feline IgG, and Asp at the amino acid position that corresponds to amino acid position 286 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 103, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Tyr at the amino acid position that corresponds to amino acid position 252 of the wild type feline IgG, and Glu at the amino acid position that corresponds to amino acid position 286 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 104, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Tyr at the amino acid position that corresponds to amino acid position 252 of the wild type feline IgG, and Leu at the amino acid position that corresponds to amino acid position 301 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 105, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Tyr at the amino acid position that corresponds to amino acid position 252 of the wild type feline IgG, and Val at the amino acid position that corresponds to amino acid position 301 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 106, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Tyr at the amino acid position that corresponds to amino acid position 252 of the wild type feline IgG, and Tyr at the amino acid position that corresponds to amino acid position 301 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 107, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Tyr at the amino acid position that corresponds to amino acid position 252 of the wild type feline IgG, and Val at the amino acid position that corresponds to amino acid position 309 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 108, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Tyr at the amino acid position that corresponds to amino acid position 252 of the wild type feline IgG, and Asp at the amino acid position that corresponds to amino acid position 309 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 109, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Tyr at the amino acid position that corresponds to amino acid position 252 of the wild type feline IgG, and Try at the amino acid position that corresponds to amino acid position 377 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 110, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Tyr at the amino acid position that corresponds to amino acid position 252 of the wild type feline IgG, and Leu at the amino acid position that corresponds to amino acid position 377 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 111, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Tyr at the amino acid position that corresponds to amino acid position 252 of the wild type feline IgG, and Asp at the amino acid position that corresponds to amino acid position 392 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 112, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Tyr at the amino acid position that corresponds to amino acid position 252 of the wild type feline IgG, and Glu at the amino acid position that corresponds to amino acid position 392 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 113, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Met at the amino acid position that corresponds to amino acid position 252 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 114, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Met at the amino acid position that corresponds to amino acid position 252 of the wild type feline IgG, and Asp at the amino acid position that corresponds to amino acid position 286 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 115, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Met at the amino acid position that corresponds to amino acid position 252 of the wild type feline IgG, and Glu at the amino acid position that corresponds to amino acid position 286 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 116, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Met at the amino acid position that corresponds to amino acid position 252 of the wild type feline IgG, and Leu at the amino acid position that corresponds to amino acid position 301 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 117, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Met at the amino acid position that corresponds to amino acid position 252 of the wild type feline IgG, and Val at the amino acid position that corresponds to amino acid position 301 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 118, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Met at the amino acid position that corresponds to amino acid position 252 of the wild type feline IgG, and Tyr at the amino acid position that corresponds to amino acid position 301 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 119, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Met at the amino acid position that corresponds to amino acid position 252 of the wild type feline IgG, and Val at the amino acid position that corresponds to amino acid position 309 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 120, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Met at the amino acid position that corresponds to amino acid position 252 of the wild type feline IgG, and Asp at the amino acid position that corresponds to amino acid position 309 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 121, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Met at the amino acid position that corresponds to amino acid position 252 of the wild type feline IgG, and Val at the amino acid position that corresponds to amino acid position 311 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 122, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Met at the amino acid position that corresponds to amino acid position 252 of the wild type feline IgG, and Try at the amino acid position that corresponds to amino acid position 377 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 123, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Met at the amino acid position that corresponds to amino acid position 252 of the wild type feline IgG, and Leu at the amino acid position that corresponds to amino acid position 377 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 124, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Met at the amino acid position that corresponds to amino acid position 252 of the wild type feline IgG, and Asp at the amino acid position that corresponds to amino acid position 392 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 125, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Met at the amino acid position that corresponds to amino acid position 252 of the wild type feline IgG, and Glu at the amino acid position that corresponds to amino acid position 392 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 126, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Met at the amino acid position that corresponds to amino acid position 428 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 127, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Met at the amino acid position that corresponds to amino acid position 428 of the wild type feline IgG, and Asp at the amino acid position that corresponds to amino acid position 286 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 128, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Met at the amino acid position that corresponds to amino acid position 428 of the wild type feline IgG, and Glu at the amino acid position that corresponds to amino acid position 286 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 129, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Met at the amino acid position that corresponds to amino acid position 428 of the wild type feline IgG, and Leu at the amino acid position that corresponds to amino acid position 301 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 130, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Met at the amino acid position that corresponds to amino acid position 428 of the wild type feline IgG, and Val at the amino acid position that corresponds to amino acid position 301 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 131, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Met at the amino acid position that corresponds to amino acid position 428 of the wild type feline IgG, and Tyr at the amino acid position that corresponds to amino acid position 301 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 132, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Met at the amino acid position that corresponds to amino acid position 428 of the wild type feline IgG, and Val at the amino acid position that corresponds to amino acid position 309 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 133, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Met at the amino acid position that corresponds to amino acid position 428 of the wild type feline IgG, and Glu at the amino acid position that corresponds to amino acid position 309 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 134, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Met at the amino acid position that corresponds to amino acid position 428 of the wild type feline IgG, and Asp at the amino acid position that corresponds to amino acid position 309 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 135, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Met at the amino acid position that corresponds to amino acid position 428 of the wild type feline IgG, and Val at the amino acid position that corresponds to amino acid position 311 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 136, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Met at the amino acid position that corresponds to amino acid position 428 of the wild type feline IgG, and Try at the amino acid position that corresponds to amino acid position 377 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 137, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Met at the amino acid position that corresponds to amino acid position 428 of the wild type feline IgG, and Leu at the amino acid position that corresponds to amino acid position 377 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 138, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Met at the amino acid position that corresponds to amino acid position 428 of the wild type feline IgG, and Asp at the amino acid position that corresponds to amino acid position 392 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 139, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Met at the amino acid position that corresponds to amino acid position 428 of the wild type feline IgG, and Glu at the amino acid position that corresponds to amino acid position 392 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 140, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Leu at the amino acid position that corresponds to amino acid position 428 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 141, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Leu at the amino acid position that corresponds to amino acid position 428 of the wild type feline IgG, and Asp at the amino acid position that corresponds to amino acid position 286 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 142, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Leu at the amino acid position that corresponds to amino acid position 428 of the wild type feline IgG, and Leu at the amino acid position that corresponds to amino acid position 301 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 143, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Leu at the amino acid position that corresponds to amino acid position 428 of the wild type feline IgG, and Val at the amino acid position that corresponds to amino acid position 301 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 144, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Leu at the amino acid position that corresponds to amino acid position 428 of the wild type feline IgG, and Tyr at the amino acid position that corresponds to amino acid position 301 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 145, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Leu at the amino acid position that corresponds to amino acid position 428 of the wild type feline IgG, and Asp at the amino acid position that corresponds to amino acid position 309 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 146, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Leu at the amino acid position that corresponds to amino acid position 428 of the wild type feline IgG, and Try at the amino acid position that corresponds to amino acid position 377 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 147, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Leu at the amino acid position that corresponds to amino acid position 428 of the wild type feline IgG, and Leu at the amino acid position that corresponds to amino acid position 377 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 148, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Leu at the amino acid position that corresponds to amino acid position 428 of the wild type feline IgG, and Asp at the amino acid position that corresponds to amino acid position 392 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 149, and the light chain comprises an amino acid sequence having at least 80% (e.g., 85%, 90%, 95%, 97%, 98%, 99%, and 100%) sequence identity to SEQ ID NO: 100.

In some embodiments, the polypeptide comprises Leu at the amino acid position that corresponds to amino acid position 428 of the wild type feline IgG, and Glu at the amino acid position that corresponds to amino acid position 392 of the wild type feline IgG. In some embodiments, the heavy chain comprises an amino acid sequence having at least 80% (e.g., 85%, 90%, 95%, 97%, 98%, 99%, and 100%) sequence identity to SEQ ID NO: 150, and the light chain comprises an amino acid sequence having at least 80% (e.g., 85%, 90%, 95%, 97%, 98%, 99%, and 100%) sequence identity to SEQ ID NO: 100.

In some embodiments, the therapeutic protein is any protein described herein. In some embodiments, the polypeptide or polypeptides further comprises a feline IgG CH2 domain, IgG CH3 domain, or IgG Fc region as described herein. The modified feline IgG CH2 domain, IgG CH3 domain, or IgG Fc region can enhance the half-life the therapeutic proteins in vivo.

### Pharmaceutical Compositions

In one aspect, the invention features a pharmaceutical composition comprising (i) any of the polypeptides disclosed herein, and (ii) a pharmaceutically acceptable excipient.

To prepare pharmaceutical or sterile compositions of a polypeptide or polypeptides described herein, the polypeptide or polypeptides can be admixed with a pharmaceutically acceptable carrier or excipient. (See, e.g., Remington's Pharmaceutical Sciences and U.S. Pharmacopeia: National Formulary, Mack Publishing Company, Easton, Pa. (1984)).

Formulations of therapeutic and diagnostic agents may be prepared by mixing with acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.). In one embodiment, the polypeptide or polypeptides of the present invention are diluted to an appropriate concentration in a sodium acetate solution pH 5-6, and NaCl or sucrose is added for tonicity. Additional agents, such as polysorbate 20 or polysorbate 80, may be added to enhance stability.

Toxicity and therapeutic efficacy of the polypeptide compositions, administered alone or in combination with another agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index (LD₅₀/ED₅₀). In particular aspects, a polypeptide or polypeptides exhibiting high therapeutic indices are desirable. The data obtained from these cell culture assays and animal studies can be used in formulating a range of dosage for use in felines. The dosage of such compounds lies preferably within a range of circulating concentrations that include the ED₅₀ with little or no toxicity. The dosage may vary within this range depending upon the dosage form employed and the route of administration.

Any suitable mode of administration can be used. Exemplary suitable routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial. In some embodiments, the polypeptide or polypeptides can be administered by an invasive route such as by injection. In further embodiments, the polypeptide or polypeptides is administered intravenously, subcutaneously, intramuscularly, intraarterially, intratumorally, or by inhalation, aerosol delivery.

The pharmaceutical compositions disclosed herein may also be administered by infusion. Examples of well-known implants and modules form administering pharmaceutical compositions include: U.S. Pat. No. 4,487,603, which discloses an implantable micro-infusion pump for dispensing medication at a controlled rate; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments. Many other such implants, delivery systems, and modules are well known to those skilled in the art.

Alternatively, one may administer the polypeptide or polypeptides in a local rather than systemic manner, for example, via injection of the antibody directly into an arthritic joint or pathogen-induced lesion characterized by immunopathology, often in a depot or sustained release formulation. Furthermore, one may administer the polypeptide or polypeptides in a targeted drug delivery system, for example, in a liposome coated with a tissue-specific antibody, targeting, for example, arthritic joint or pathogen-induced lesion characterized by immunopathology. The liposomes will be targeted to and taken up selectively by the afflicted tissue.

The administration regimen depends on several factors, including, without limitation, the age, weight, and physical condition of the feline being treated, the serum or tissue turnover rate of the therapeutic antibody, the level of symptoms, the immunogenicity of the therapeutic polypeptide or polypeptides, and the accessibility of the target cells in the biological matrix. Preferably, the administration regimen delivers sufficient therapeutic polypeptide or polypeptides to effect improvement in the target disease state, while simultaneously minimizing undesired side effects. Accordingly, the amount of biologic delivered depends in part on the particular therapeutic polypeptide or polypeptides and the severity of the condition being treated. Guidance in selecting appropriate doses of therapeutic antibodies is available (see, e.g., Wawrzynczak Antibody Therapy, Bios Scientific Pub. Ltd, Oxfordshire, U K (1996); Milgrom et al. New Engl. J. Med. 341: 1966-1973 (1999); Slamon et al. New Engl. J. Med. 344: 783-792 (2001); Beniaminovitz et al. New Engl. J. Med. 342: 613-619 (2000); Ghosh et al. New Engl. J. Med. 348: 24-32 (2003); Lipsky et al. New Engl. J. Med. 343: 1594-1602 (2000)).

Determination of the appropriate dose of the polypeptide or polypeptides is made by one skilled in the art, e.g., using parameters or factors known or suspected in the art to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced.

### Nucleic Acids, Vectors, Host Cells, and Methods of Making

The disclosure also encompasses nucleic acid or nucleic acids encoding the polypeptide or polypeptides described herein, a vector or vectors comprising the nucleic acid or nucleic acids, and host cells comprising the nucleic acid or nucleic acids or the vector or vectors.

In one aspect, the invention features a nucleic acid or nucleic acids encoding any of the polypeptides disclosed herein.

In another aspect, the invention features an expression vector or expression vectors comprising a nucleic acid or nucleic acids encoding any of the polypeptides disclosed herein.

In another aspect, the invention features a host cell comprising a nucleic acid or nucleic acids encoding any of the polypeptides disclosed herein, or an expression vector or expression vectors comprising a nucleic acid or nucleic acids encoding any of the polypeptides disclosed herein.

In another aspect, the invention features a method of making a polypeptide, the method comprising:
(i) providing a nucleic acid or nucleic acids encoding any of the polypeptides disclosed herein;
(ii) expressing the nucleic acid or nucleic acids in a host cell culture, thereby producing the polypeptide; and, optionally,
(iii) collecting the polypeptide produced in (ii) from the host cell culture.

The polypeptide or polypeptides described herein may be produced in bacterial or eukaryotic cells. Some polypeptides, e.g., Fabs, can be produced in bacterial cells, e.g., E. coli cells. Polypeptides can also be produced in eukaryotic cells such as transformed cell lines (e.g., CHO, 293E, COS, 293T, Hela). In addition, polypeptides (e.g., scFvs) can be expressed in a yeast cell such as Pichia (see, e.g., Powers et al., J Immunol Methods. 251: 123-35 (2001)), Hanseula, or Saccharomyces. To produce the antibody of interest, a polynucleotide or polynucleotides encoding the polypeptide or polypeptides is/are constructed, introduced into an expression vector or expression vectors, and then expressed in suitable host cells. To improve expression, the nucleotide sequences of the genes can be recoded without changing (or minimally changing—e.g., removal of a C-terminal residue of the heavy or light chain) the amino acid sequence. The areas for potential recoding include those associated with translation initiation, codon usage, and possible unintended mRNA splicing. Polynucleotides encoding an Fc region variant described herein would be readily envisioned by the ordinarily skilled artisan.

Standard molecular biology techniques can be used to prepare the recombinant expression vector(s), transfect the host cells, select for transformants, culture the host cells, and recover the polypeptide (e.g., antibody).

If the polypeptide or polypeptides is to be expressed in bacterial cells (e.g., E. coli), the expression vector may have characteristics that permit amplification of the vector in the bacterial cells. Additionally, when E. coli such as JM109, DH5a, HB101, or XL1-Blue is used as a host, the vector may have a promoter, for example, a lacZ promoter (Ward et al., 341: 544-546 (1989), araB promoter (Better et al., Science, 240: 1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), PEGFP, and pET (when this expression vector is used, the host is preferably BL21 expressing T7 RNA polymerase). The expression vector may contain a signal sequence for antibody secretion. For production into the periplasm of E. coli, the pelB signal sequence (Lei et al., J. Bacteriol., 169: 4379 (1987)) may be used as the signal sequence for antibody secretion. For bacterial expression, calcium chloride methods or electroporation methods may be used to introduce the expression vector into the bacterial cell.

If the polypeptide or polypeptides is to be expressed in animal cells such as CHO, COS, and NIH3T3 cells, the expression vector may include a promoter for expression in these cells, for example, an SV40 promoter (Mulligan et al., Nature, 277: 108 (1979)) (e.g., early simian virus 40 promoter), MMLV-LTR promoter, EF1a promoter (Mizushima et al., Nucleic Acids Res., 18: 5322 (1990)), or CMV promoter (e.g., human cytomegalovirus immediate early promoter). In addition to the nucleic acid sequence encoding the Fc region variant, the recombinant expression vectors may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665, and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

In some embodiments, the polypeptide or polypeptides are produced in mammalian cells. Exemplary mammalian host cells for expressing polypeptide or polypeptides include Chinese Hamster Ovary (CHO cells) (including dhfr-CHO cells, described in Urlaub and Chasin (1980), Proc. Natl. Acad. Sci. USA, 77: 4216-4220, used with a DHFR selectable marker, e.g., as described in Kaufman and Sharp (1982), Mol. Biol. 159: 601 621), human embryonic kidney 293 cells (e.g., 293, 293E, 293T), COS cells, NIH3T3 cells, lymphocytic cell lines, e.g., NSO myeloma cells and SP2 cells, and a cell from a transgenic animal, e.g., a transgenic mammal. For example, the cell is a mammary epithelial cell.

In an exemplary system for antibody expression, a recombinant expression vector encoding both the antibody heavy chain and the antibody light chain of the antibody is introduced into dhfr-CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and the antibody is recovered from the culture medium.

### Methods of Treatment

The polypeptide or polypeptides disclosed herein can be used to treat or prevent any disease or disorder in a cat in need thereof. This invention is particularly helpful in the treatment of chronic conditions where repeated dosing is required. Because of the increased half-life of the protein therapeutic, less frequent dosing and/or reduced dose levels may be possible.

In one aspect, the invention features a method of treating or preventing a feline disease or disorder in a cat in need thereof, the method comprising administering an effective amount of a composition comprising any of the polypeptides disclosed herein, or a pharmaceutical composition comprising (i) any of the polypeptides disclosed herein, and (ii) a pharmaceutically acceptable excipient.

In another aspect, the invention features any of the polypeptides disclosed herein, or the pharmaceutical composition comprising (i) any of the polypeptides disclosed herein, and (ii) a pharmaceutically acceptable excipient, for use in treatment or prevention of a feline disease or disorder in a cat in need thereof.

Any suitable feline disease or disorder may be treated. In some embodiments, the feline disease or disorder is an allergic disease, a chronic pain, an acute pain, an inflammatory disease, an autoimmune disease, an endocrine disease, a gastrointestinal disease, a cardiovascular disease, a renal disease, a fertility related disorder, an infectious disease, or a cancer.

In other embodiments, the feline disease or disorder is atopic dermatitis, allergic dermatitis, osteoarthritic pain, arthritis, anemia, or obesity.

In some embodiments, the disease, disorder, condition, or symptoms being treated or prevented is an allergic disease, a chronic pain, an acute pain, an inflammatory disease, an autoimmune disease, an endocrine disease, a gastrointestinal disease, a skeletal/musculoskeletal disease, a cardiovascular disease, a neurological disease, a renal disease, a metabolic disease, an immunological disease, a genetic/inherited disease, a fertility related disorder, an infectious disease, or a cancer. In certain embodiments, the disease or disorder being treated or prevented is atopic dermatitis, allergic dermatitis, food allergy, osteoarthritic pain, perioperative pain, dental pain, cancer pain, arthritis, anemia, obesity, or diabetes.

Antibodies may not only be used to treat or prevent disease but also to modulate normal biological function, for example, to manage fertility or behavior.

In some embodiments, the polypeptide or polypeptides disclosed herein, or the pharmaceutical composition comprising the polypeptide or polypeptides disclosed herein, is administered parenterally, by subcutaneous administration, intravenous infusion, or intramuscular injection. In some embodiments, the polypeptide or polypeptides disclosed herein, or the pharmaceutical composition comprising the polypeptide or polypeptides disclosed herein, is administered as a bolus injection or by continuous infusion over a period of time. In some embodiments, the polypeptide or polypeptides disclosed herein, or the pharmaceutical composition comprising the polypeptide or polypeptides disclosed herein, is administered by an intramuscular, an intraperitoneal, an intracerebrospinal, a subcutaneous, an intra-arterial, an intrasynovial, an intrathecal, or an inhalation route.

In some embodiments, the polypeptide or polypeptides disclosed herein, or the pharmaceutical composition comprising the polypeptide or polypeptides disclosed herein, is administered in an amount in the range of 0.01 mg/kg body weight to 50 mg/kg body weight per dose. In some embodiments, the polypeptide or polypeptides disclosed herein, or the pharmaceutical composition comprising the polypeptide or polypeptides disclosed herein, is administered 0.01 to 55 mg/kg, 0.01 to 50 mg/kg, 0.01 to 45 mg/kg, 0.01 to 40 mg/kg, 0.01 to 35 mg/kg, 0.01 to 30 mg/kg, 0.01 to 25 mg/kg, 0.01 to 20 mg/kg, 0.01 to 15 mg/kg, 0.01 to 10 mg/kg, 0.01 to 5 mg/kg, or 0.01 to 1 mg/kg administered daily, weekly, monthly, every two months, every three months, every four months, every five months, or every six months, for example. One exemplary dosage of the antibody would be in the range from 0.01 mg/kg to 10 mg/kg. Thus, one or more doses of 0.01 mg/kg, 0.02 mg/kg, 0.04 mg/kg, 0.1 mg/kg, 0.2 mg/kg, 0.4 mg/kg, 1.0 mg/kg, 2.0 mg/kg, 4.0 mg/kg, or 10 mg/kg (or any combination thereof) may be administered to the animal. In some embodiments, the polypeptide or polypeptides disclosed herein, or the pharmaceutical composition comprising the polypeptide or polypeptides disclosed herein, is administered 2 mg/kg body weight per dose.

In some embodiments, the polypeptide or polypeptides disclosed herein, or the pharmaceutical composition comprising the polypeptide or polypeptides disclosed herein, is administered within one, two, three, four, five, or six months, or within one, two, or three weeks, of each other. In some embodiments, the polypeptide or polypeptides disclosed herein, or the pharmaceutical composition comprising the polypeptide or polypeptides disclosed herein, is administered every week. In some embodiments, the polypeptide or polypeptides disclosed herein, or the pharmaceutical composition comprising the polypeptide or polypeptides disclosed herein, is administered every two weeks. In some embodiments, the polypeptide or polypeptides disclosed herein, or the pharmaceutical composition comprising the polypeptide or polypeptides disclosed herein, is administered every three weeks. In some embodiments, the polypeptide or polypeptides disclosed herein, or the pharmaceutical composition comprising the polypeptide or polypeptides disclosed herein, is administered every month. In some embodiments, the polypeptide or polypeptides disclosed herein, or the pharmaceutical composition comprising the polypeptide or polypeptides disclosed herein, is administered every two months. In some embodiments, the polypeptide or polypeptides disclosed herein, or the pharmaceutical composition comprising the polypeptide or polypeptides disclosed herein, is administered every three months. In some embodiments, the polypeptide or polypeptides disclosed herein, or the pharmaceutical composition comprising the polypeptide or polypeptides disclosed herein, is administered every four months. In some embodiments, the polypeptide or polypeptides disclosed herein, or the pharmaceutical composition comprising the polypeptide or polypeptides disclosed herein, is administered every five months. In some embodiments, the polypeptide or polypeptides disclosed herein, or the pharmaceutical composition comprising the polypeptide or polypeptides disclosed herein, is administered every six months. In some embodiments, the polypeptide or polypeptides disclosed herein, or the pharmaceutical composition comprising the polypeptide or polypeptides disclosed herein, is administered to a cat at one time or over a series of treatments. In some embodiments, the dose is administered once per week for at least two or three consecutive weeks, and in some embodiments, this cycle of treatment is repeated two or more times, optionally interspersed with one or more weeks of no treatment.

In some embodiments, the polypeptide or polypeptides disclosed herein, or the pharmaceutical composition comprising the polypeptide or polypeptides disclosed herein, is administered in combination, concurrently, sequentially, or in conjunction with one or more further therapeutic agents. In some embodiments, the polypeptide or polypeptides disclosed herein, or the pharmaceutical composition comprising the polypeptide or polypeptides disclosed herein, is administered in combination, with one or more further therapeutic agents. In some embodiments, the polypeptide or polypeptides disclosed herein, or the pharmaceutical composition comprising the polypeptide or polypeptides disclosed herein, is administered concurrently with one or more further therapeutic agents. In some embodiments, the polypeptide or polypeptides disclosed herein, or the pharmaceutical composition comprising the polypeptide or polypeptides disclosed herein, is administered sequentially with one or more further therapeutic agents. In some embodiments, the polypeptide or polypeptides disclosed herein, or the pharmaceutical composition comprising the polypeptide or polypeptides disclosed herein, is administered in conjunction with one or more further therapeutic agents. Any suitable further therapeutic agents may be used.

### Diagnosis

The polypeptide or polypeptides disclosed herein can also be used for various diagnostic purposes, for example, to determine whether a cat has any particular disease or disorder. In some embodiments, the polypeptide or polypeptides may comprise a binding domain. The binding domain can specifically bind to a protein, subunit, domain, motif, and/or epitope as described herein (e.g., a maker for cancer cells). In some embodiments the polypeptide or polypeptides further comprises a labeling group. In general, label groups fall into a variety of classes, depending on the assay in which they are to be detected: a) isotopic labels, which may be radioactive or heavy isotopes; b) magnetic labels (e.g., magnetic particles); c) redox active moieties; d) optical dyes; enzymatic groups (e.g., horseradish peroxidase, ß-galactosidase, luciferase, alkaline phosphatase); e) biotinylated groups; and f) predetermined polypeptide epitopes recognized by a secondary reporter (e.g., leucine zipper pair sequences, binding sites for secondary antibodies, metal binding domains, epitope tags, etc.). In some embodiments, the labelling group is coupled to the antibody via spacer arms of various lengths to reduce potential steric hindrance. Various methods for labelling proteins are known in the art and may be used in performing the present invention.

In some embodiments, the labeling group is a probe, a dye (e.g., a fluorescent dye), or a radioactive isotope (e.g., ³H, ¹⁴C, ²²Na, ³⁶Cl, ³⁵S, ³³P, or ¹²⁵I).

Specific labels can also include optical dyes, including, but not limited to, chromophores, phosphors and fluorophores, with the latter being specific in many instances. Fluorophores can be either “small molecule” fluores, or proteinaceous fluores.

The fluorescent label can be any molecule that may be detected via its inherent fluorescent properties. Suitable fluorescent labels include, but are not limited to, fluorescein, rhodamine, tetramethylrhodamine, eosin, erythrosin, coumarin, methyl-coumarins, pyrene, Malachite green, stilbene, Lucifer Yellow, Cascade BlueJ, Texas Red, IAEDANS, EDANS, BODIPY FL, LC Red 640, Cy 5, Cy 5.5, LC Red 705, Oregon green, the Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R-phycoerythrin (PE) (Molecular Probes, Eugene, Oreg.), FITC, Rhodamine, and Texas Red (Pierce, Rockford, III.), Cy5, Cy5.5, Cy7 (Amersham Life Science, Pittsburgh, Pa.). Suitable optical dyes, including fluorophores, are described in Molecular Probes Handbook by Richard P. Haugland, which is incorporated by reference in its entirety.

Suitable proteinaceous fluorescent labels also include, but are not limited to, green fluorescent protein, including a Renilla, Ptilosarcus, or Aequorea species of GFP (Chalfie et al., 1994, Science, 263: 802-805), EGFP (Clontech Laboratories, Inc., Genbank Accession Number U55762), blue fluorescent protein (BFP, Quantum Biotechnologies, Inc. 1801 de Maisonneuve Blvd. West, 8th Floor, Montreal, Quebec, Canada H3H1J9; Stauber, 1998, Biotechniques, 24: 462-471; Heim et al., 1996, Curr. Biol., 6: 178-182), enhanced yellow fluorescent protein (EYFP, Clontech Laboratories, Inc.), luciferase (Ichiki et al., 1993, J. Immunol., 150: 5408-5417), ß galactosidase (Nolan et al., 1988, Proc. Natl. Acad. Sci. USA, 85: 2603-2607) and Renilla (WO 92/15673, WO 95/07463, WO 98/14605, WO 98/26277, WO 99/49019, U.S. Pat. Nos. 5,292,658, 5,418,155, 5,683,888, 5,741,668, 5,777,079, 5,804,387, 5,874,304, 5,876,995, and 5,925,558). All of the above-cited references in this paragraph are expressly incorporated herein by reference in the entirety.

### Assays

### FcγRI and FcγRIII Binding:

Binding to FcγRI and FcγRIII is a measure of the ability of an antibody to mediate ADCC. In order to assess this property for an antibody an assay to measure binding of the antibody to FcγRI and FcγRIII can be conducted using methods known in the art.

### C1q Binding:

Binding to the first component of complement, C1q, is a measure of the ability of an antibody to mediate CDC. In order to assess this property for an antibody, an assay to measure binding of the antibody to C1q can be conducted using methods known in the art.

### Half-Life:

Methods of measuring half-life of an antibody are well known in the art. See, e.g., Booth et al., MAbs, 10(7): 1098-1110 (2018). Exemplary animal models include non-human primate models and transgenic mouse models. The transgenic mouse models can be null for mouse FcRn alpha chain and express the feline FcRn alpha transgene (e.g., under the control of a constitutive promoter). The feline FcRn alpha chain can pair in vivo with the mouse 32-microglobulin protein forming a functional chimeric FcRn heterodimer. As an example, the half-life of an antibody (e.g., a feline antibody) can be measured by injection of the antibody into a cat model and measuring levels of the antibody in the serum over a certain period of time.

### EXAMPLES

### Example 1: Surface Plasmon Resonance Analysis Using Biacore™ 8K

For the surface plasmon resonance (SPR) analyses using the Biacore™ 8K, bovine serum albumin (BSA) is immobilized to CM5 sensor chip. The sensor chip surface of flow cells 1 and 2 are activated by freshly mixed 50 mmol/L N-Hydroxysuccinimide and 200 mmol/L 1-ethyl-3-(3-dimethylaminopropyl) carbodiimide hydrochloride for 420 s (10 μL/min). Afterwards, BSA diluted in 10 mM sodium acetate (pH 4.5) is injected into the flow cell 2 to achieve conjugation, while flow cell 1 is set as blank. After the amine coupling reaction, the remaining active coupling sites on chip surface are blocked with 420 s injection of 1 mM ethanolamine hydrochloride. The running buffer for the binding experiment is HBS-EP (10 mM HEPES, 500 mM NaCl, 3 mM EDTA, 0.05% Tween 20, pH 5.5) and it is run at 25° C. Supernatants from the variants are injected over chip surface and captured via the SASA (single-domain antibody against serum albumin) tag (See, e.g., US 2013/0129727A1) onto the immobilized BSA for 60 sec. Feline FcRn (GenBank KF773786 (IgG receptor FcRn large subunit p51) and European Nucleotide Archive AY829266.1 (feline beta-2-microglobulin)) at 200 nM is injected for 120 sec and the dissociation is complete with running buffer for 120 sec. The flow rate for the immobilization phase of BSA was 10 μl/min and the flow rate for the association and dissociation phase is 30 μl/min. All of the data is processed using the Biacore™ 8K evaluation software version 1.1.

### Example 2: Binding Kinetics of Feline IgG1a Variants to Feline FcRn Using C1 Biosensors

Feline IgG1a variants (S252Y, S252M, T286D, T286E, R301L, R301V, R301Y, L309V, L309E, Q311V, 1377Y, 1377L, R392D, R392E, S428M, S428L, S252Y+T286D, S252Y+T286E, S252Y+R301L, S252Y+R301V, S252Y+R301Y, S252Y+L309V, S252Y+L309D, S252Y+1377Y, S252Y+1377L, S252Y+R392D, S252Y+R392E, S252M+T286D, S252M+T286E, S252M+R301L, S252M+R301V, S252M+R301Y, S252M+L309V, S252M+L309D, S252M+Q311V, S252M+1377Y, S252M+1377L, S252M+R392D, S252M+R392E, S428M+T286D, S428M+T286E, S428M+R301L, S428M+R301V, S428M+R301Y, S428M+L309V, S428M+L309E, S428M+L309D, S428M+Q311V, S428M+1377Y, S428M+1377L, S428M+R392D, S428M+R392E, S428L+T286D, S428L+R301L, S428L+R301V, S428L+R301Y, S428L+L309D, S428L+1377Y, S428L+1377L, S428L+R392D, S428L+R392E, and wild type) are evaluated for binding kinetics to feline FcRn (GenBank KF773786 (feline FcRn large subunit p51) and European Nucleotide Archive AY829266.1 (feline beta-2-microglulin)) at pH 6.0. EU numbering is used to identify the positions. In this study, the feline Fc variants carrying single amino acid substitutions or a combination of amino acid substitutions are synthesized into the feline IgG1a (Kanai et al., 2000, Vet. Immunol. Immunopathol. 73: 53) using the variable domain described by Gearing D P et al. (2016, J Vet Intern Med, 30: 1129). The synthesized feline IgGa variant DNAs are subcloned into a mammalian expression vector and transiently transfected into CHO cells. The conditioned media are purified using protein A chromatography.

For the feline FcRn binding experiments, all assays are completed on a Biacore™ 8K+ system at 25° C. In this set of experiments, antibodies are immobilized using standard amine coupling reagents to Series S C1 sensor chips. A mixture of 200 mmol/L 1-ethyl-3-(3-dimethylaminopropyl) carbodiimide hydrochloride (EDC) and 50 mmol/L N-Hydroxysuccinimide (NHS) is injected for 420 seconds to activate the surface. Then, antibodies are injected at a concentration of 0.5 to 2 μg/ml in 10 mM sodium acetate pH 5.0 for 120 seconds. Finally, 1 M ethanolamine is injected for 420 seconds. The running buffer is 1× phosphate buffered saline (PBS)-P+(Cytiva, Cat #28995084) adjusted to pH 6.0.

To evaluate the binding affinity of the feline IgG1a variants to feline FcRn at pH 6.0, a range of concentrations from 1.56-2000 nM of feline FcRn are chosen and injected in single cycle mode.

Four concentrations per antibody are injected at 5 μl/min for 90 seconds, followed by 180 seconds dissociation. Each concentration series is injected three times in this format, with at least three buffer-only cycles for proper reference subtraction. The surface is regenerated with two injections of 1×PBS-P+, pH 7.4 for 30 seconds, followed by a 60 second wait command. Three startup cycles are included to stabilize the surface prior to analysis.

Data are evaluated using Insight Evaluation Software by fitting to a 1:1 kinetic interaction model, or by fitting to steady state affinity. Quality metrics including the U-value and T-value are used to select the accepted parameters. A U-value of less than 15 is considered acceptable for kinetic rate constants, while a T-value of greater than 100 is considered acceptable for kinetic rate constants. Where these values are outside the range, the steady state affinity parameters are considered acceptable.

It is expected that all of the variants, as well as the wild type, will not bind to feline FcRn at pH 7.4, and that the variants tested will show increased affinity for feline FcRn at acidic pH (e.g., pH 5.5 or pH 6.0) as compared to wild type Fc.

### Example 3: Pharmacokinetic Study of Feline IgG1a Fc Variants with Increased Fcrn Binding and Wild Type Feline IgG1a

A pharmacokinetic (PK) study is undertaken with male and female cats. Feline IgG1a Fc variants, including the antibody carrying a wild type feline IgG1a Fc domain (SEQ ID NO: 1), are prepared using the anti-NGF variable domain as previously described by Gearing et al. (2016, J Vet Intern Med, 30: 1129).

The animals are randomized so that each group contains an equal number of males and females. Each animal is administered with single intravenous dose of 2 mg/kg of antibody. Approximately 0.5 ml of whole blood is collected at the following time points: 0 (pre-dose), 4 hours, and 1, 2, 4, 6, 10, 14, 18, 22, 30, 34 38, 42 days post injection. Serum is separated from whole blood and assayed for the presence of the antibody by an ELISA that is specific for anti-NGF antibodies. Serum concentrations of six anti-NGF monoclonal antibody (mAb) variants are described with a two-compartmental pharmacokinetic (PK) model with linear clearance using non-linear mixed effects (NLME) modelling. Population parameters are estimated using the stochastic approximation of expectation-maximization (SAEM) algorithm implemented in Monolix Suite 2019R1 (Monolix version 2019R1. Antony, France: Lixoft SAS, 2019). Individual parameters are modeled as random variables with log-normal distributions. PK parameters depend on body weight (BW) using mAb-typical coefficients (β_{BW,Cl}=0.75, β_{BW,V1}=β_{BW,V2}=1, β_{BW,Q}=2/3). The equation (Dong et al. 2011. Clin Pharmacokinet, 50: 131) for an individual parameter φᵢ is:

$${φ}_{i} = {{φ}_{pop} ( \frac{{BW}_{i}}{{BW}_{ref}} )}^{β} {e}^{η}$$

where φₚₒₚ is the population typical parameter, η is a random variable with mean 0 and standard deviation ω, BWᵢ is the body weight of animal i, and BW_{ref} is the reference body weight of 2 kg.

Antibody variants are discriminated by using a categorial covariate on clearance, inter-compartmental exchange coefficient, and peripheral volume according to the equation:

$${φ}_{i} = {φ}_{pop} {e}^{( {βΩ}_{i} )} {e}^{η}$$

where Ωᵢ=1 if the individual variant covariate is in the category and 22; =0 otherwise. The wild type (WT) mAb variant is used as a reference. It is expected that the antibodies containing feline IgG1a Fc variants will have increased terminal half-life as compared to those containing wild type feline IgG1a Fc.

### Example 4: Pharmacokinetic Study of Feline IgG1a Fc Variants with Two or Three Fc Substitutions and Wild Type Feline IgG1a

A pharmacokinetic (PK) study is undertaken with male and female cats. Feline IgG1a Fc variants, including the antibody carrying a wild type feline IgG1a Fc domain, are prepared using the anti-NGF variable domain as previously described by Gearing et al. (2016, J Vet Intern Med, 30: 1129). The feline IgG1a variants tested in this study include: S252Y+T286D, S252Y+T286E, S252Y+R301L, S252Y+R301V, S252Y+R301Y, S252Y+L309V, S252Y+L309D, S252Y+1377Y, S252Y+1377L, S252Y+R392D, S252Y+R392E, S252M+T286D, S252M+T286E, S252M+R301L, S252M+R301V, S252M+R301Y, S252M+L309V, S252M+L309D, S252M+Q311V, S252M+1377Y, S252M+1377L, S252M+R392D, S252M+R392E, S428M+T286D, S428M+T286E, S428M+R301L, S428M+R301V, S428M+R301Y, S428M+L309V, S428M+L309E, S428M+L309D, S428M+Q311V, S428M+1377Y, S428M+1377L, S428M+R392D, S428M+R392E, S428L+T286D, S428L+R301L, S428L+R301V, S428L+R301Y, S428L+L309D, S428L+1377Y, S428L+1377L, S428L+R392D, S428L+R392E, and wild type.

The animals are randomized into groups with equal number of males and females. Each animal is administered with a single intravenous dose of 2 mg/kg of antibody. Approximately 0.5 ml of whole blood is collected at the following time points: 0 (pre-dose), 4 hours, and 1, 2, 4, 6, 10, 14, 18, 22, 30, 34 38, 42 days post injection. Serum is separated from whole blood and assayed for the presence of the antibody by an ELISA that is specific for feline anti-NGF antibodies. Serum concentrations of the seven anti-NGF monoclonal antibody (mAb) variants are described with a two-compartmental pharmacokinetic (PK) model with linear clearance using non-linear mixed effects (NLME) modelling (Population parameters are estimated using the stochastic approximation of expectation-maximization (SAEM) algorithm implemented in Monolix Suite 2019R1 (Monolix version 2019R1. Antony, France: Lixoft SAS, 2019)). Individual parameters are modeled as random variables with log-normal distributions. PK parameters depend on body weight (BW) using mAb-typical coefficients (β_{BW,Cl}=0.75, β_{BW,V1}=β_{BW,V2}=1, β_{BW,Q}=2/3). The equation (Dong et al. 2011. Clin Pharmacokinet, 50: 131) for an individual parameter φᵢ is:

$${φ}_{i} = {{φ}_{pop} ( \frac{{BW}_{i}}{{BW}_{ref}} )}^{β} {e}^{η}$$

where φₚₒₚ is the population typical parameter, n is a random variable with mean 0 and standard deviation ω, BWᵢ is the body weight of animal i, and BW_{ref} is the reference body weight of 2 kg.

Antibody variants are discriminated by using a categorial covariate on clearance, inter-compartmental exchange coefficient, and peripheral volume according to the equation:

$${φ}_{i} = {φ}_{pop} {e}^{( {βΩ}_{i} )} {e}^{η}$$

where Ωᵢ=1 if the individual variant covariate is in the category and Ωᵢ=0 otherwise. The wild type (WT) mAb is used as a reference.

It is expected that the combination of amino acid substitutions in the IgG Fc region will markedly improve the terminal half-life of the anti-NGF IgG1a antibodies in feline in vivo, when compared to anti-NGF IgG1a antibodies carrying (i) a wild type feline IgG1a Fc region or (ii) a feline IgG1a Fc variant with only a single amino acid substitution.

### Example 5: Binding Kinetics of Feline IgG Fc Variants to Feline FcRn

A set of feline Fc variants was expressed in an IgG format and purified. The IgGs comprised a light chain comprising the amino acid sequence of SEQ ID NO: 100, and a heavy chain comprising the amino acid sequence of any one of SEQ ID NOs: 101-150. The variable domain for the heavy and light chains for the IgGs were described in International Patent Application Publication No. WO 2023/97275, which is incorporated herein by reference in its entirety. The feline IgG1a constant domain contained the MALA mutations (M234A and L244A, according to EU numbering) which reduces potential effector activity (ADCC and CDC). The heavy and light chains were synthesized and subcloned into PCDNA™3.4 vector (Thermo Fisher Scientific) with a signal sequence at the N-terminus of the chains. The heavy and light chain constructs were co-transfected in EXPICHO™ cells and incubated for 7 days before the conditioned medium was purified using MABSELECT™ SURE™ protein A resin. The purified antibodies were buffer exchanged in PBS, pH 7.4. The FcRn complex consists of a large subunit (p51) and a small subunit (B2-microglobulin, p14), and the feline FcRn protein was generated by co-expressing these two proteins in CHO cells. The soluble portion of feline FcRn large subunit p51 isoform X1 (NCBI Reference Sequence No. XP_044901959.1) with a 6×His-tag (HHHHHH, SEQ ID NO: 153) at the C-terminus and a signal peptide (MGWSCIILFLVATATGVHS, SEQ ID NO: 154) at the N-terminus is shown in SEQ ID NO: 151. The feline B2-microglobulin (NCBI Reference Sequence No. NP_001009876.1) with a signal peptide (MGWSCIILFLVATATGVHS, SEQ ID NO: 154) at the N-terminus and a STREP-TAG®II (WSHPQFEK, SEQ ID NO: 155) at the C-terminus is shown in SEQ ID NO: 152. The conditioned medium from the transfected CHO cells was purified using HISTRAP™ FF chromatography and formulated in PBS, PH 7.2. By analytical size exclusion chromatography using a TSKGEL® G3000SWxi column, the feline FcRn was >95% pure.

The feline FcRn binding experiments at pH 5.9 were completed on a BIACORE™ T200 instrument. A Series S Protein L Sensor chip (Cytiva, Cat. No. BR29205137) was used to capture the antibody variants via the kappa (κ) light chain. The feline variants were captured onto the protein L chip at a flow rate of 10 μL/min for 60 seconds. 1×PBX-P+(Cytiva Cat 28995084) was adjusted to pH 5.9 for the running buffer. The feline FcRn was flowed over the sensor chip at 30 μL/min for 120 seconds contact time and 600 seconds dissociation time. Regeneration of the flow cells was completed by flowing over 10 mM glycine, pH 1.7 at 30 μL/min for 30 seconds. The data were evaluated by the BIACORE™T200 Evaluation software v3.2.1 by fitting to a 1:1 kinetic interaction model. The kinetic binding data for the feline IgG variants at pH 5.9 are shown in Table 6 below.

TABLE 6
Binding data for IgGs with feline
Fc variant to feline FcRn at pH 5.9

| Fc or Fc Variant | ka (1/Ms) | kd (1/s) | KD (M) | Rmax (RU) |
|---|---|---|---|---|
| S252Y | 7.42E+05 | 2.68E−02 | 3.61E−08 | 210.55 |
| S252Y, T286D | 6.31E+05 | 9.55E−03 | 1.52E−08 | 248.65 |
| S252Y, T286E | 6.50E+05 | 7.83E−03 | 1.21E−08 | 270.55 |
| S252Y, R301L | 7.14E+05 | 2.77E−02 | 3.88E−08 | 227.3 |
| S252Y, R301V | 7.24E+05 | 2.80E−02 | 3.87E−08 | 156.1 |
| S252Y, R301Y | 6.96E+05 | 2.62E−02 | 3.76E−08 | 64.1 |
| S252Y, L309V | 7.47E+05 | 1.39E−02 | 1.87E−08 | 73.3 |
| S252Y, L309D | 5.02E+05 | 2.84E−02 | 5.67E−08 | 181.3 |
| S252Y, 1377Y | 7.16E+05 | 2.58E−02 | 3.61E−08 | 158 |
| S252Y, 1377L | 7.12E+05 | 2.24E−02 | 3.15E−08 | 133.9 |
| S252Y- R392D | 1.19E+06 | 2.96E−02 | 2.50E−08 | 57.7 |
| S252Y, R392E | 1.19E+06 | 2.87E−02 | 2.41E−08 | 101.9 |
| S252M | 6.90E+05 | 1.92E−01 | 2.79E−07 | 139.15 |
| S252M, T286D | 8.50E+05 | 1.05E−01 | 1.23E−07 | 47 |
| S252M, T286E | 7.15E+05 | 7.19E−02 | 1.01E−07 | 136.05 |
| S252M, R301L | 1.20E+06 | 3.32E−01 | 2.80E−07 | 144.9 |
| S252M, R301V | 7.45E+05 | 2.06E−01 | 2.78E−07 | 131.45 |
| S252M, R301Y | 6.95E+05 | 1.80E−01 | 2.58E−07 | 135.1 |
| S252M, L309V | 9.58E+05 | 1.24E−01 | 1.29E−07 | 112.1 |
| S252M, L309D | 5.81E+05 | 2.03E−01 | 3.52E−07 | 59.6 |
| S252M, Q311V | 8.65E+05 | 1.21E−01 | 1.52E−07 | 72.4 |
| S252M, 1377Y | 8.62E+05 | 1.75E−01 | 2.02E−07 | 50.3 |
| S252M, 1377L | 8.69E+05 | 1.87E−01 | 2.16E−07 | 65.45 |
| S252M, R392D | 8.56E+05 | 2.29E−01 | 2.68E−07 | 66.4 |
| S252M, R392E | 9.65E+05 | 1.77E−01 | 1.86E−07 | 197.10 |
| S428M | 1.01E+06 | 7.02E−02 | 7.01E−08 | 249.05 |
| S428M, T286D | 1.07E+06 | 3.72E−02 | 3.49E−08 | 236.55 |
| S428M, T286E | 1.07E+06 | 2.92E−02 | 2.74E−08 | 227.75 |
| S428M, R301L | 8.09E+05 | 8.35E−02 | 1.03E−07 | 75.95 |
| S428M, R301V | 8.12E+05 | 8.01E−02 | 9.85E−08 | 120.8 |
| S428M, R301Y | 8.30E+05 | 8.20E−02 | 9.87E−08 | 86 |
| S428M, L309V | 1.15E+06 | 3.58E−02 | 3.13E−08 | 240.65 |
| S428M, L309E | 9.05E+05 | 4.13E−02 | 4.56E−08 | 248.70 |
| S428M, L309D | 5.92E+05 | 1.04E−01 | 1.77E−07 | 95.4 |
| S428M, Q311V | 1.12E+06 | 3.19E−02 | 2.84E−08 | 236.40 |
| S428M, 1377Y | 9.00E+05 | 6.36E−02 | 7.08E−08 | 75.4 |
| S428M, 1377L | 9.81E+05 | 7.51E−02 | 7.62E−08 | 117.1 |
| S428M, R392D | 7.89E+05 | 7.95E−02 | 1.01E−07 | 135.1 |
| S428M, R392E | 7.84E+05 | 7.66E−02 | 9.78E−08 | 124.25 |
| S428L | 9.68E+05 | 6.19E−02 | 6.40E−08 | 64.90 |
| S428L, T286D | 1.00E+06 | 3.33E−02 | 3.34E−08 | 237.05 |
| S428L, R301L | 1.01E+06 | 1.16E−01 | 1.15E−07 | 96.2 |
| S428L, R301V | 7.63E+05 | 8.32E−02 | 1.09E−07 | 124.7 |
| S428L, R301Y | 7.12E+05 | 7.06E−02 | 9.91E−08 | 106.45 |
| S428L, L309D | 5.97E+05 | 1.20E−01 | 2.00E−07 | 168.8 |
| S428L, 1377Y | 1.09E+06 | 1.17E−01 | 1.07E−07 | 91.9 |
| S428L, 1377L | 8.19E+05 | 7.23E−02 | 8.77E−08 | 155.9 |
| S428L, R392D | 9.04E+05 | 9.34E−02 | 1.04E−07 | 85.9 |
| S428L, R392E | 8.01E+05 | 7.72E−02 | 9.65E−08 | 136.95 |
| Wild-type Fc | 7.09E+05 | 2.73E−01 | 4.17E−07 | 158.90 |

In conclusion, these data show that the feline Fc variants tested have superior FcRn binding properties compared with wild type feline Fc. For example, feline Fc variants with S252Y/T286D, S252Y/T286E, S252Y/L309V, S252M/T286D, S252M/T286E, S252M/L309V, S252M/Q311V, S252M/R392E, S428M/T286D, S428M/T286E, S428M/L309V, S428M/L309E, S428M/Q311V, and S428L/T286D substitutions have improved affinity to feline FcRn compared with wild type Fc or feline Fc variants with single amino acid substitution (e.g., S252Y, S252M, S428M, or S428L).

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A polypeptide comprising a feline IgG Fc region variant, wherein the feline IgG Fc region variant comprises (a) Tyr at a position that corresponds to amino acid position 252 of a wild type feline IgG, and (b) at least one amino acid substitution at a position selected from the group consisting of:
(i) a position that corresponds to amino acid position 286 of a wild type feline IgG;
(ii) a position that corresponds to amino acid position 301 of a wild type feline IgG;
(iii) a position that corresponds to amino acid position 309 of a wild type feline IgG, wherein the amino acid substitution is Asp or Val;
(iv) a position that corresponds to amino acid position 377 of a wild type feline IgG; and
(v) a position that corresponds to amino acid position 392 of a wild type feline IgG;
wherein the amino acid positions are based on EU numbering, and wherein the polypeptide has increased binding affinity to feline FcRn compared to an Fc domain of the wild type feline IgG.

2. The polypeptide of claim 1, wherein the polypeptide comprises:
(i) Asp or Glu at the amino acid position that corresponds to amino acid position 286 of the wild type feline IgG;
(ii) Leu, Tyr, or Val at the amino acid position that corresponds to amino acid position 301 of the wild type feline IgG;
(iii) Leu or Tyr at the amino acid position that corresponds to amino acid position 377 of the wild type feline IgG; and/or
(iv) Asp or Glu at the amino acid position that corresponds to amino acid position 392 of the wild type feline IgG.

3. The polypeptide of claim 1, wherein the polypeptide comprises:
(i) Tyr at the amino acid position that corresponds to amino acid position 252 of the wild type feline IgG and Asp at the amino acid position that corresponds to amino acid position 286 of the wild type feline IgG;
(ii) Tyr at the amino acid position that corresponds to amino acid position 252 of the wild type feline IgG and Glu at the amino acid position that corresponds to amino acid position 286 of the wild type feline IgG;
(iii) Tyr at the amino acid position that corresponds to amino acid position 252 of the wild type feline IgG and Leu at the amino acid position that corresponds to amino acid position 301 of the wild type feline IgG;
(iv) Tyr at the amino acid position that corresponds to amino acid position 252 of the wild type feline IgG and Tyr at the amino acid position that corresponds to amino acid position 301 of the wild type feline IgG;
(v) Tyr at the amino acid position that corresponds to amino acid position 252 of the wild type feline IgG and Val at the amino acid position that corresponds to amino acid position 301 of the wild type feline IgG;
(vi) Tyr at the amino acid position that corresponds to amino acid position 252 of the wild type feline IgG and Asp at the amino acid position that corresponds to amino acid position 309 of the wild type feline IgG;
(vii) Tyr at the amino acid position that corresponds to amino acid position 252 of the wild type feline IgG and Val at the amino acid position that corresponds to amino acid position 309 of the wild type feline IgG;
(viii) Tyr at the amino acid position that corresponds to amino acid position 252 of the wild type feline IgG and Leu at the amino acid position that corresponds to amino acid position 377 of the wild type feline IgG;
(ix) Tyr at the amino acid position that corresponds to amino acid position 252 of the wild type feline IgG and Tyr at the amino acid position that corresponds to amino acid position 377 of the wild type feline IgG;
(x) Tyr at the amino acid position that corresponds to amino acid position 252 of the wild type feline IgG and Asp at the amino acid position that corresponds to amino acid position 392 of the wild type feline IgG; or
(xi) Tyr at the amino acid position that corresponds to amino acid position 252 of the wild type feline IgG and Glu at the amino acid position that corresponds to amino acid position 392 of the wild type feline IgG.

4. A polypeptide comprising a feline IgG Fc region variant, wherein the feline IgG Fc region variant comprises (a) Met at a position that corresponds to amino acid position 252 of a wild type feline IgG, and (b) at least one amino acid substitution at a position selected from the group consisting of:
(i) a position that corresponds to amino acid position 286 of a wild type feline IgG;
(ii) a position that corresponds to amino acid position 301 of a wild type feline IgG;
(iii) a position that corresponds to amino acid position 309 of a wild type feline IgG, wherein the amino acid substitution is Asp or Val;
(iv) a position that corresponds to amino acid position 311 of a wild type feline IgG, wherein the amino acid substitution is Val;
(v) a position that corresponds to amino acid position 377 of a wild type feline IgG; and
(vi) a position that corresponds to amino acid position 392 of a wild type feline IgG;
wherein the amino acid positions are based on EU numbering, and wherein the polypeptide has increased binding affinity to feline FcRn compared to an Fc domain of the wild type feline IgG.

5. The polypeptide of claim 4, wherein the polypeptide comprises:
(i) Asp or Glu at the amino acid position that corresponds to amino acid position 286 of the wild type feline IgG;
(ii) Leu, Tyr, or Val at the amino acid position that corresponds to amino acid position 301 of the wild type feline IgG;
(iii) Val at the amino acid position that corresponds to amino acid position 311 of the wild type feline IgG;
(iv) Leu or Tyr at the amino acid position that corresponds to amino acid position 377 of the wild type feline IgG; and/or
(v) Asp or Glu at the amino acid position that corresponds to amino acid position 392 of the wild type feline IgG.

6. The polypeptide of claim 4, wherein the polypeptide comprises:
(i) Met at the amino acid position that corresponds to amino acid position 252 of the wild type feline IgG and Asp at the amino acid position that corresponds to amino acid position 286 of the wild type feline IgG;
(ii) Met at the amino acid position that corresponds to amino acid position 252 of the wild type feline IgG and Glu at the amino acid position that corresponds to amino acid position 286 of the wild type feline IgG;
(iii) Met at the amino acid position that corresponds to amino acid position 252 of the wild type feline IgG and Leu at the amino acid position that corresponds to amino acid position 301 of the wild type feline IgG;
(iv) Met at the amino acid position that corresponds to amino acid position 252 of the wild type feline IgG and Tyr at the amino acid position that corresponds to amino acid position 301 of the wild type feline IgG;
(v) Met at the amino acid position that corresponds to amino acid position 252 of the wild type feline IgG and Val at the amino acid position that corresponds to amino acid position 301 of the wild type feline IgG;
(vi) Met at the amino acid position that corresponds to amino acid position 252 of the wild type feline IgG and Asp at the amino acid position that corresponds to amino acid position 309 of the wild type feline IgG;
(vii) Met at the amino acid position that corresponds to amino acid position 252 of the wild type feline IgG and Val at the amino acid position that corresponds to amino acid position 309 of the wild type feline IgG;
(viii) Met at the amino acid position that corresponds to amino acid position 252 of the wild type feline IgG and Val at the amino acid position that corresponds to amino acid position 311 of the wild type feline IgG;
(ix) Met at the amino acid position that corresponds to amino acid position 252 of the wild type feline IgG and Leu at the amino acid position that corresponds to amino acid position 377 of the wild type feline IgG;
(x) Met at the amino acid position that corresponds to amino acid position 252 of the wild type feline IgG and Tyr at the amino acid position that corresponds to amino acid position 377 of the wild type feline IgG;
(xi) Met at the amino acid position that corresponds to amino acid position 252 of the wild type feline IgG and Asp at the amino acid position that corresponds to amino acid position 392 of the wild type feline IgG; or
(xii) Met at the amino acid position that corresponds to amino acid position 252 of the wild type feline IgG and Glu at the amino acid position that corresponds to amino acid position 392 of the wild type feline IgG.

7. A polypeptide comprising a feline IgG Fc region variant, wherein the feline IgG Fc region variant comprises (a) Met at a position that corresponds to amino acid position 428 of a wild type feline IgG, and (b) at least one amino acid substitution at a position selected from the group consisting of:
(i) a position that corresponds to amino acid position 286 of a wild type feline IgG;
(ii) a position that corresponds to amino acid position 301 of a wild type feline IgG;
(iii) a position that corresponds to amino acid position 309 of a wild type feline IgG;
(iv) a position that corresponds to amino acid position 311 of a wild type feline IgG, wherein the amino acid substitution is Val;
(v) a position that corresponds to amino acid position 377 of a wild type feline IgG; and
(vi) a position that corresponds to amino acid position 392 of a wild type feline IgG;
wherein the amino acid positions are based on EU numbering, and wherein the polypeptide has increased binding affinity to feline FcRn compared to an Fc domain of the wild type feline IgG.

8. The polypeptide of claim 7, wherein the polypeptide comprises:
(i) Asp or Glu at the amino acid position that corresponds to amino acid position 286 of the wild type feline IgG;
(ii) Leu, Tyr, or Val at the amino acid position that corresponds to amino acid position 301 of the wild type feline IgG;
(iii) Asp, Glu, or Val at the amino acid position that corresponds to amino acid position 309 of the wild type feline IgG;
(iv) Val at the amino acid position that corresponds to amino acid position 311 of the wild type feline IgG;
(v) Leu or Tyr at the amino acid position that corresponds to amino acid position 377 of the wild type feline IgG; and/or
(vi) Asp or Glu at the amino acid position that corresponds to amino acid position 392 of the wild type feline IgG.

9. The polypeptide of claim 7, wherein the polypeptide comprises:
(i) Met at the amino acid position that corresponds to amino acid position 428 of the wild type feline IgG and Asp at the amino acid position that corresponds to amino acid position 286 of the wild type feline IgG;
(ii) Met at the amino acid position that corresponds to amino acid position 428 of the wild type feline IgG and Glu at the amino acid position that corresponds to amino acid position 286 of the wild type feline IgG;
(iii) Met at the amino acid position that corresponds to amino acid position 428 of the wild type feline IgG and Leu at the amino acid position that corresponds to amino acid position 301 of the wild type feline IgG;
(iv) Met at the amino acid position that corresponds to amino acid position 428 of the wild type feline IgG and Tyr at the amino acid position that corresponds to amino acid position 301 of the wild type feline IgG;
(v) Met at the amino acid position that corresponds to amino acid position 428 of the wild type feline IgG and Val at the amino acid position that corresponds to amino acid position 301 of the wild type feline IgG;
(vi) Met at the amino acid position that corresponds to amino acid position 428 of the wild type feline IgG and Asp at the amino acid position that corresponds to amino acid position 309 of the wild type feline IgG;
(vii) Met at the amino acid position that corresponds to amino acid position 428 of the wild type feline IgG and Glu at the amino acid position that corresponds to amino acid position 309 of the wild type feline IgG;
(viii) Met at the amino acid position that corresponds to amino acid position 428 of the wild type feline IgG and Val at the amino acid position that corresponds to amino acid position 309 of the wild type feline IgG;
(ix) Met at the amino acid position that corresponds to amino acid position 428 of the wild type feline IgG and Val at the amino acid position that corresponds to amino acid position 311 of the wild type feline IgG;
(x) Met at the amino acid position that corresponds to amino acid position 428 of the wild type feline IgG and Leu at the amino acid position that corresponds to amino acid position 377 of the wild type feline IgG;
(xi) Met at the amino acid position that corresponds to amino acid position 252 of the wild type feline IgG and Tyr at the amino acid position that corresponds to amino acid position 377 of the wild type feline IgG;
(xii) Met at the amino acid position that corresponds to amino acid position 252 of the wild type feline IgG and Asp at the amino acid position that corresponds to amino acid position 392 of the wild type feline IgG; or
(xiii) Met at the amino acid position that corresponds to amino acid position 252 of the wild type feline IgG and Glu at the amino acid position that corresponds to amino acid position 392 of the wild type feline IgG.

10. A polypeptide comprising a feline IgG Fc region variant, wherein the feline IgG Fc region variant comprises (a) Leu at a position that corresponds to amino acid position 428 of a wild type feline IgG, and (b) at least one amino acid substitution at a position selected from the group consisting of:
(i) a position that corresponds to amino acid position 286 of a wild type feline IgG;
(ii) a position that corresponds to amino acid position 301 of a wild type feline IgG;
(iii) a position that corresponds to amino acid position 309 of a wild type feline IgG, wherein the amino acid substitution is Asp;
(iv) a position that corresponds to amino acid position 377 of a wild type feline IgG; and
(v) a position that corresponds to amino acid position 392 of a wild type feline IgG;
wherein the amino acid positions are based on EU numbering, and wherein the polypeptide has increased binding affinity to feline FcRn compared to an Fc domain of the wild type feline IgG.

11. The polypeptide of claim 10, wherein the polypeptide comprises:
(i) Asp at the amino acid position that corresponds to amino acid position 286 of the wild type feline IgG;
(ii) Leu, Tyr, or Val at the amino acid position that corresponds to amino acid position 301 of the wild type feline IgG;
(iii) Asp at the amino acid position that corresponds to amino acid position 309 of the wild type feline IgG;
(iv) Leu or Tyr at the amino acid position that corresponds to amino acid position 377 of the wild type feline IgG; and/or
(v) Asp or Glu at the amino acid position that corresponds to amino acid position 392 of the wild type feline IgG.

12. The polypeptide of claim 10, wherein the polypeptide comprises:
(i) Leu at the amino acid position that corresponds to amino acid position 428 of the wild type feline IgG and Asp at the amino acid position that corresponds to amino acid position 286 of the wild type feline IgG;
(ii) Leu at the amino acid position that corresponds to amino acid position 428 of the wild type feline IgG and Leu at the amino acid position that corresponds to amino acid position 301 of the wild type feline IgG;
(iii) Leu at the amino acid position that corresponds to amino acid position 428 of the wild type feline IgG and Tyr at the amino acid position that corresponds to amino acid position 301 of the wild type feline IgG;
(iv) Leu at the amino acid position that corresponds to amino acid position 428 of the wild type feline IgG and Val at the amino acid position that corresponds to amino acid position 301 of the wild type feline IgG;
(v) Leu at the amino acid position that corresponds to amino acid position 428 of the wild type feline IgG and Asp at the amino acid position that corresponds to amino acid position 309 of the wild type feline IgG;
(vi) Leu at the amino acid position that corresponds to amino acid position 428 of the wild type feline IgG and Leu at the amino acid position that corresponds to amino acid position 377 of the wild type feline IgG;
(vii) Leu at the amino acid position that corresponds to amino acid position 252 of the wild type feline IgG and Tyr at the amino acid position that corresponds to amino acid position 377 of the wild type feline IgG;
(viii) Leu at the amino acid position that corresponds to amino acid position 252 of the wild type feline IgG and Asp at the amino acid position that corresponds to amino acid position 392 of the wild type feline IgG; or
(ix) Leu at the amino acid position that corresponds to amino acid position 252 of the wild type feline IgG and Glu at the amino acid position that corresponds to amino acid position 392 of the wild type feline IgG.

13. The polypeptide of claim 1, wherein the wild type feline IgG is a feline IgG1a comprising an Fc domain having an amino acid sequence of SEQ ID NO: 1, a feline IgG1b comprising an Fc domain having an amino acid sequence of SEQ ID NO: 2, or a feline IgG2 comprising an Fc domain having an amino acid sequence of SEQ ID NO: 3.

14. The polypeptide of claim 1, wherein the polypeptide binds to the feline FcRn at a higher level at an acidic pH than at a neutral pH.

15. The polypeptide of claim 14, wherein the polypeptide binds to the feline FcRn at a higher level at a pH of 5.5 to 6.0 than at pH 7.4.

16. A pharmaceutical composition comprising (i) the polypeptide of claim 1, and (ii) a pharmaceutically acceptable excipient.

17. A nucleic acid or nucleic acids encoding the polypeptide of claim 1.

18. An expression vector or expression vectors comprising the nucleic acid or nucleic acids of claim 17.

19. A host cell comprising the expression vector or expression vectors of claim 18.

20. A method of making a polypeptide, the method comprising:
(i) providing a nucleic acid or nucleic acids of claim 17; and
(ii) expressing the nucleic acid or nucleic acids in a host cell culture, thereby producing the polypeptide.


SEQUENCE LISTING (161 sequences)

SEQ ID NO: 1 - protein, 219 aa, Felis catus
PPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 2 - protein, 219 aa, Felis catus
PPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKDKGQPHEPQVYVLPPAQEELSRNKVSVTCLIEGFYPSDIAVEWEITGQPEPENNYRTTPPQLDSDGTYFLYSRLSVDRSRWQRGNTYTCSVSHEALHSHHTQKSLTQSPGK

SEQ ID NO: 3 - protein, 219 aa, Felis catus
VPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 4 - protein, 110 aa, Felis catus
PPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAK

SEQ ID NO: 5 - protein, 110 aa, Felis catus
PPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKDK

SEQ ID NO: 6 - protein, 110 aa, Felis catus
VPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAK

SEQ ID NO: 7 - protein, 109 aa, Felis catus
GQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 8 - protein, 109 aa, Felis catus
GQPHEPQVYVLPPAQEELSRNKVSVTCLIEGFYPSDIAVEWEITGQPEPENNYRTTPPQLDSDGTYFLYSRLSVDRSRWQRGNTYTCSVSHEALHSHHTQKSLTQSPGK

SEQ ID NO: 9 - protein, 109 aa, Felis catus
GQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 10 - protein, 17 aa, Felis catus
KTDHPPGPKPCDCPKCP

SEQ ID NO: 11 - protein, 17 aa, Felis catus
KTDHPPGPKPCDCPKCP

SEQ ID NO: 12 - protein, 17 aa, Felis catus
KTASTIESKTGEGPKCP

SEQ ID NO: 13 - protein, 4 aa, synthetic construct
GGGS

SEQ ID NO: 14 - protein, 4 aa, synthetic construct
SGGG

SEQ ID NO: 15 - protein, 5 aa, synthetic construct
GGGGS

SEQ ID NO: 16 - protein, 5 aa, synthetic construct
SGGGG

SEQ ID NO: 17 - protein, 6 aa, synthetic construct
GGGGGS

SEQ ID NO: 18 - protein, 6 aa, synthetic construct
SGGGGG

SEQ ID NO: 19 - protein, 7 aa, synthetic construct
GGGGGGS

SEQ ID NO: 20 - protein, 7 aa, synthetic construct
SGGGGGG

SEQ ID NO: 21 - protein, 9 aa, synthetic construct
GDSITSGYW

SEQ ID NO: 22 - protein, 16 aa, synthetic construct
YISYSGITDYNPSLKS

SEQ ID NO: 23 - protein, 12 aa, synthetic construct
ARYGNYGYAMDY

SEQ ID NO: 24 - protein, 15 aa, synthetic construct
RASESVDTYGNSFMH

SEQ ID NO: 25 - protein, 7 aa, synthetic construct
RASNLES

SEQ ID NO: 26 - protein, 9 aa, synthetic construct
QQSYEDPWT

SEQ ID NO: 27 - protein, 16 aa, synthetic construct
YISYSGITYYNPSLKS

SEQ ID NO: 28 - protein, 15 aa, synthetic construct
RASESVDTYGNSFIH

SEQ ID NO: 29 - protein, 10 aa, synthetic construct
GYTFTSYVMH

SEQ ID NO: 30 - protein, 17 aa, synthetic construct
YINPKNDGTFYNGKFKG

SEQ ID NO: 31 - protein, 7 aa, synthetic construct
FNYGIAY

SEQ ID NO: 32 - protein, 11 aa, synthetic construct
RASQEISGYLS

SEQ ID NO: 33 - protein, 7 aa, synthetic construct
AASTLDS

SEQ ID NO: 34 - protein, 9 aa, synthetic construct
VQYASYPWT

SEQ ID NO: 35 - protein, 17 aa, synthetic construct
YINPNNDGTFYNGKFKG

SEQ ID NO: 36 - protein, 7 aa, synthetic construct
FYYGFAY

SEQ ID NO: 37 - protein, 9 aa, synthetic construct
LQYASYPWT

SEQ ID NO: 38 - protein, 5 aa, synthetic construct
SSWMN

SEQ ID NO: 39 - protein, 17 aa, synthetic construct
QIYPGDGDTNYNGKFKG

SEQ ID NO: 40 - protein, 10 aa, synthetic construct
ARHYDGSTDY

SEQ ID NO: 41 - protein, 11 aa, synthetic construct
RASENIYSNLA

SEQ ID NO: 42 - protein, 7 aa, synthetic construct
AATNLAD

SEQ ID NO: 43 - protein, 9 aa, synthetic construct
QHFWGTPYT

SEQ ID NO: 44 - protein, 7 aa, synthetic construct
FSSYGMH

SEQ ID NO: 45 - protein, 11 aa, synthetic construct
VISYDGSIKYY

SEQ ID NO: 46 - protein, 14 aa, synthetic construct
TGEYSGYDTDPQYS

SEQ ID NO: 47 - protein, 11 aa, synthetic construct
RASQGIGDDLG

SEQ ID NO: 48 - protein, 7 aa, synthetic construct
GTSTLQS

SEQ ID NO: 49 - protein, 9 aa, synthetic construct
LQDSNYPLT

SEQ ID NO: 50 - protein, 7 aa, synthetic construct
GYIFITY

SEQ ID NO: 51 - protein, 6 aa, synthetic construct
FPASGS

SEQ ID NO: 52 - protein, 12 aa, synthetic construct
GDGNYALDAMDY

SEQ ID NO: 53 - protein, 15 aa, synthetic construct
RASESVDSYGNSFMH

SEQ ID NO: 54 - protein, 7 aa, synthetic construct
LASNLES

SEQ ID NO: 55 - protein, 9 aa, synthetic construct
QQNNEDPLT

SEQ ID NO: 56 - protein, 8 aa, synthetic construct
GFTLTQYG

SEQ ID NO: 57 - protein, 9 aa, synthetic construct
VIWATGATD

SEQ ID NO: 58 - protein, 13 aa, synthetic construct
DGWWYATSWYFDV

SEQ ID NO: 59 - protein, 11 aa, synthetic construct
KASQDINHYLN

SEQ ID NO: 60 - protein, 7 aa, synthetic construct
YTSRLHS

SEQ ID NO: 61 - protein, 9 aa, synthetic construct
QQGDHFPRT

SEQ ID NO: 62 - protein, 5 aa, synthetic construct
DYGMH

SEQ ID NO: 63 - protein, 17 aa, synthetic construct
YISSGSRAVFFADTVKG

SEQ ID NO: 64 - protein, 10 aa, synthetic construct
DRYDGRGFAY

SEQ ID NO: 65 - protein, 11 aa, synthetic construct
RASQSISNNLH

SEQ ID NO: 66 - protein, 7 aa, synthetic construct
YASQSIS

SEQ ID NO: 67 - protein, 9 aa, synthetic construct
QQSNSWPLT

SEQ ID NO: 68 - protein, 5 aa, synthetic construct
SYAMS

SEQ ID NO: 69 - protein, 17 aa, synthetic construct
YISSGGDYIYYADTVKG

SEQ ID NO: 70 - protein, 9 aa, synthetic construct
DPITGTFAY

SEQ ID NO: 71 - protein, 11 aa, synthetic construct
RASQDINNYLN

SEQ ID NO: 72 - protein, 7 aa, synthetic construct
YTSTLHS

SEQ ID NO: 73 - protein, 9 aa, synthetic construct
QQGNTLPWT

SEQ ID NO: 74 - protein, 17 aa, synthetic construct
YISSGGDYFYYADTVKG

SEQ ID NO: 75 - protein, 11 aa, synthetic construct
RASQDITNYLN

SEQ ID NO: 76 - protein, 9 aa, synthetic construct
QQGHMLPWT

SEQ ID NO: 77 - protein, 5 aa, synthetic construct
DYYMA

SEQ ID NO: 78 - protein, 17 aa, synthetic construct
NINYDGSSTYYLDSLKS

SEQ ID NO: 79 - protein, 8 aa, synthetic construct
GLTWDFDV

SEQ ID NO: 80 - protein, 11 aa, synthetic construct
KASQDVDTAVA

SEQ ID NO: 81 - protein, 7 aa, synthetic construct
LASTRHT

SEQ ID NO: 82 - protein, 9 aa, synthetic construct
QQYSRFPLT

SEQ ID NO: 83 - protein, 5 aa, synthetic construct
NNNVN

SEQ ID NO: 84 - protein, 17 aa, synthetic construct
GVWAGGATDYNSALKGR

SEQ ID NO: 85 - protein, 14 aa, synthetic construct
DGGYSSSTLYAMDA

SEQ ID NO: 86 - protein, 11 aa, synthetic construct
RASEDIYNALA

SEQ ID NO: 87 - protein, 7 aa, synthetic construct
NTDTLHT

SEQ ID NO: 88 - protein, 8 aa, synthetic construct
HYFHYPRT

SEQ ID NO: 89 - protein, 5 aa, synthetic construct
SYTIH

SEQ ID NO: 90 - protein, 17 aa, synthetic construct
NINPTSGYTENNQRFKD

SEQ ID NO: 91 - protein, 13 aa, synthetic construct
WGFKYDGEWSFDV

SEQ ID NO: 92 - protein, 11 aa, synthetic construct
RASQGISIWLS

SEQ ID NO: 93 - protein, 7 aa, synthetic construct
KASNLHI

SEQ ID NO: 94 - protein, 9 aa, synthetic construct
LQSQTYPLT

SEQ ID NO: 95 - protein, 5 aa, synthetic construct
YYDIN

SEQ ID NO: 96 - protein, 17 aa, synthetic construct
WIFPGDGGTKYNETFKG

SEQ ID NO: 97 - protein, 14 aa, synthetic construct
ARGGTSVIRDAMDY

SEQ ID NO: 98 - protein, 15 aa, synthetic construct
RASESVDNYGISFMH

SEQ ID NO: 99 - protein, 9 aa, synthetic construct
QQSNKDPLT

SEQ ID NO: 100 - protein, 237 aa, synthetic construct
METDTLLLWVLLLWVPGSTGEIQMTQSPTSLSASVGDRVTITCRASEGIANNLSWYQQTPGKAPKLLIYATSNLHSGVPSRFSGSGSGTDFTLTISSLQPEDFATYYCQQGFKWPLTFGGGTKLEITRSDAQPSVFLFQPSLDELHTGSASIVCILNDFYPKEVNVKWKVDGVVQNKGIQESTTEQNSKDSTYSLSSTLTMSSTEYQSHEKFSCEVTHKSLASTLVKSFNRSECQRE

SEQ ID NO: 101 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPG

SEQ ID NO: 102 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLYISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPG

SEQ ID NO: 103 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLYISRTPEVTCLVVDLGPDDSDVQITWFVDNTQVYDAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPG

SEQ ID NO: 104 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLYISRTPEVTCLVVDLGPDDSDVQITWFVDNTQVYEAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPG

SEQ ID NO: 105 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLYISRTPEVTCLVVDLGPDDSDVQITWFVDNTQVYTAKTSPREEQFNSTYLVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPG

SEQ ID NO: 106 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLYISRTPEVTCLVVDLGPDDSDVQITWFVDNTQVYTAKTSPREEQFNSTYVVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPG

SEQ ID NO: 107 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLYISRTPEVTCLVVDLGPDDSDVQITWFVDNTQVYTAKTSPREEQFNSTYYVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPG

SEQ ID NO: 108 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLYISRTPEVTCLVVDLGPDDSDVQITWFVDNTQVYTAKTSPREEQFNSTYRVVSVLPIVHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPG

SEQ ID NO: 109 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLYISRTPEVTCLVVDLGPDDSDVQITWFVDNTQVYTAKTSPREEQFNSTYRVVSVLPIDHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPG

SEQ ID NO: 110 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLYISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDYAVEWEITGQPEPENNYRTTPPQLDSDGTYFVYSKLSVDRSHWQRGNTYTCSVSHEALHSHHTQKSLTQSPG

SEQ ID NO: 111 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLYISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDLAVEWEITGQPEPENNYRTTPPQLDSDGTYFVYSKLSVDRSHWQRGNTYTCSVSHEALHSHHTQKSLTQSPG

SEQ ID NO: 112 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLYISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYDTTPPQLDSDGTYFVYSKLSVDRSHWQRGNTYTCSVSHEALHSHHTQKSLTQSPG

SEQ ID NO: 113 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLYISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYETTPPQLDSDGTYFVYSKLSVDRSHWQRGNTYTCSVSHEALHSHHTQKSLTQSPG

SEQ ID NO: 114 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLMISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPG

SEQ ID NO: 115 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLMISRTPEVTCLVVDLGPDDSDVQITWFVDNTQVYDAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPG

SEQ ID NO: 116 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLMISRTPEVTCLVVDLGPDDSDVQITWFVDNTQVYEAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPG

SEQ ID NO: 117 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLMISRTPEVTCLVVDLGPDDSDVQITWFVDNTQVYTAKTSPREEQFNSTYLVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPG

SEQ ID NO: 118 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLMISRTPEVTCLVVDLGPDDSDVQITWFVDNTQVYTAKTSPREEQFNSTYVVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPG

SEQ ID NO: 119 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLMISRTPEVTCLVVDLGPDDSDVQITWFVDNTQVYTAKTSPREEQFNSTYYVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPG

SEQ ID NO: 120 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLMISRTPEVTCLVVDLGPDDSDVQITWFVDNTQVYTAKTSPREEQFNSTYRVVSVLPIVHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPG

SEQ ID NO: 121 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLMISRTPEVTCLVVDLGPDDSDVQITWFVDNTQVYTAKTSPREEQFNSTYRVVSVLPIDHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPG

SEQ ID NO: 122 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLMISRTPEVTCLVVDLGPDDSDVQITWFVDNTQVYTAKTSPREEQFNSTYRVVSVLPILHVDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPG

SEQ ID NO: 123 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLMISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDYAVEWEITGQPEPENNYRTTPPQLDSDGTYFVYSKLSVDRSHWQRGNTYTCSVSHEALHSHHTQKSLTQSPG

SEQ ID NO: 124 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLMISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDLAVEWEITGQPEPENNYRTTPPQLDSDGTYFVYSKLSVDRSHWQRGNTYTCSVSHEALHSHHTQKSLTQSPG

SEQ ID NO: 125 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLMISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYDTTPPQLDSDGTYFVYSKLSVDRSHWQRGNTYTCSVSHEALHSHHTQKSLTQSPG

SEQ ID NO: 126 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLMISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYETTPPQLDSDGTYFVYSKLSVDRSHWQRGNTYTCSVSHEALHSHHTQKSLTQSPG

SEQ ID NO: 127 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVMHEALHSHHTQKSLTQSPG

SEQ ID NO: 128 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLSISRTPEVTCLVVDLGPDDSDVQITWFVDNTQVYDAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVMHEALHSHHTQKSLTQSPG

SEQ ID NO: 129 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLSISRTPEVTCLVVDLGPDDSDVQITWFVDNTQVYEAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVMHEALHSHHTQKSLTQSPG

SEQ ID NO: 130 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLSISRTPEVTCLVVDLGPDDSDVQITWFVDNTQVYTAKTSPREEQFNSTYLVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVMHEALHSHHTQKSLTQSPG

SEQ ID NO: 131 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLSISRTPEVTCLVVDLGPDDSDVQITWFVDNTQVYTAKTSPREEQFNSTYVVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVMHEALHSHHTQKSLTQSPG

SEQ ID NO: 132 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLSISRTPEVTCLVVDLGPDDSDVQITWFVDNTQVYTAKTSPREEQFNSTYYVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVMHEALHSHHTQKSLTQSPG

SEQ ID NO: 133 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLSISRTPEVTCLVVDLGPDDSDVQITWFVDNTQVYTAKTSPREEQFNSTYRVVSVLPIVHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVMHEALHSHHTQKSLTQSPG

SEQ ID NO: 134 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLSISRTPEVTCLVVDLGPDDSDVQITWFVDNTQVYTAKTSPREEQFNSTYRVVSVLPIEHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVMHEALHSHHTQKSLTQSPG

SEQ ID NO: 135 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLSISRTPEVTCLVVDLGPDDSDVQITWFVDNTQVYTAKTSPREEQFNSTYRVVSVLPIDHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVMHEALHSHHTQKSLTQSPG

SEQ ID NO: 136 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLSISRTPEVTCLVVDLGPDDSDVQITWFVDNTQVYTAKTSPREEQFNSTYRVVSVLPILHVDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVMHEALHSHHTQKSLTQSPG

SEQ ID NO: 137 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDYAVEWEITGQPEPENNYRTTPPQLDSDGTYFVYSKLSVDRSHWQRGNTYTCSVMHEALHSHHTQKSLTQSPG

SEQ ID NO: 138 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDLAVEWEITGQPEPENNYRTTPPQLDSDGTYFVYSKLSVDRSHWQRGNTYTCSVMHEALHSHHTQKSLTQSPG

SEQ ID NO: 139 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYDTTPPQLDSDGTYFVYSKLSVDRSHWQRGNTYTCSVMHEALHSHHTQKSLTQSPG

SEQ ID NO: 140 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYETTPPQLDSDGTYFVYSKLSVDRSHWQRGNTYTCSVMHEALHSHHTQKSLTQSPG

SEQ ID NO: 141 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVLHEALHSHHTQKSLTQSPG

SEQ ID NO: 142 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLSISRTPEVTCLVVDLGPDDSDVQITWFVDNTQVYDAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVLHEALHSHHTQKSLTQSPG

SEQ ID NO: 143 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLSISRTPEVTCLVVDLGPDDSDVQITWFVDNTQVYTAKTSPREEQFNSTYLVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVLHEALHSHHTQKSLTQSPG

SEQ ID NO: 144 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLSISRTPEVTCLVVDLGPDDSDVQITWFVDNTQVYTAKTSPREEQFNSTYVVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVLHEALHSHHTQKSLTQSPG

SEQ ID NO: 145 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLSISRTPEVTCLVVDLGPDDSDVQITWFVDNTQVYTAKTSPREEQFNSTYYVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVLHEALHSHHTQKSLTQSPG

SEQ ID NO: 146 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLSISRTPEVTCLVVDLGPDDSDVQITWFVDNTQVYTAKTSPREEQFNSTYRVVSVLPIDHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVLHEALHSHHTQKSLTQSPG

SEQ ID NO: 147 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDYAVEWEITGQPEPENNYRTTPPQLDSDGTYFVYSKLSVDRSHWQRGNTYTCSVLHEALHSHHTQKSLTQSPG

SEQ ID NO: 148 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDLAVEWEITGQPEPENNYRTTPPQLDSDGTYFVYSKLSVDRSHWQRGNTYTCSVLHEALHSHHTQKSLTQSPG

SEQ ID NO: 149 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYDTTPPQLDSDGTYFVYSKLSVDRSHWQRGNTYTCSVLHEALHSHHTQKSLTQSPG

SEQ ID NO: 150 - protein, 477 aa, synthetic construct
METDTLLLWVLLLWVPGSTGDVQLVESGGDLVKPGGSLRLTCVASGLSLTSDSMSWVRQAPGKGLQWVSTLWSNRGTYYTDSVKGRFTISKDNAENTLYLQMNNLKTEDTATYYCASIYYYEADYLHWYFDFWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYETTPPQLDSDGTYFVYSKLSVDRSHWQRGNTYTCSVLHEALHSHHTQKSLTQSPG

SEQ ID NO: 151 - protein, 301 aa, synthetic construct
MGWSCIILFLVATATGVHSAESHLSLLYHLTAVSSPAPGTPAFWVSGWLGPQQYLSYNNMRAQAEPCGAWVWENQVSWYWEKETTDLRNKQELFLEALKVLGEGGPYTLQGLLGCELGPDNASVPVAKFALNGEDFMDFDPKLGTWSGEWPETETISKRWMQEAGAVSKERTFLLNSCPQRLLGHLERGRGNLEWKEPPSMRLKARPGSPGFSVLTCSAFSFYPPELQLRFLRNGLAAGSGEGDFGPNGDGSFHAWSSLTVKSGDEHHYRCLVQHAGLPQPLTVELESPAKSSMPHHHHHH

SEQ ID NO: 152 - protein, 125 aa, synthetic construct
MGWSCIILFLVATATGVHSVQHSPKVQVYSRHPAENGKPNFLNCYVSGFHPPQIDITLMKNGKKMEAEQTDLSFNRDWTFYLLVHTEFTPTVEDEYSCQVNHTTLSEPKVVKWDRDMWSHPQFEK

SEQ ID NO: 153 - protein, 6 aa, synthetic construct
HHHHHH

SEQ ID NO: 154 - protein, 19 aa, synthetic construct
MGWSCIILFLVATATGVHS

SEQ ID NO: 155 - protein, 8 aa, synthetic construct
WSHPQFEK

SEQ ID NO: 156 - protein, 8 aa, synthetic construct
GLSLTSDS

SEQ ID NO: 157 - protein, 7 aa, synthetic construct
LWSNRGT

SEQ ID NO: 158 - protein, 17 aa, synthetic construct
ASIYYYEADYLHWYFDF

SEQ ID NO: 159 - protein, 6 aa, synthetic construct
EGIANN

SEQ ID NO: 160 - skipped

SEQ ID NO: 161 - protein, 9 aa, synthetic construct
QQGFKWPLT